(19) United States
(12) Patent Application Publication
(10) Pub. No.: US 2024/0101707 A1
(43) Pub. Date: Mar. 28, 2024

(54) A METHOD OF PREVENTING, ALLEVIATING OR TREATING A TUMOR

(71) Applicant: JIANGSU ALPHAMAB BIOPHARMACEUTICALS CO., LTD., Suzhou (CN)

(72) Inventors: Ting XU, Suzhou (CN); Junfang XU, Suzhou (CN); Pilin WANG, Suzhou (CN)

(73) Assignee: JIANGSU ALPHAMAB BIOPHARMACEUTICALS CO., LTD., Suzhou (CN)

(21) Appl. No.: 18/454,006

(22) Filed: Aug. 22, 2023

Related U.S. Application Data
(63) Continuation of application No. PCT/CN2022/077313, filed on Feb. 22, 2022.

(30) Foreign Application Priority Data
Feb. 23, 2021 (WO) PCT/CN221/077490

(51) Int. Cl. C07K 16/32; A61K 31/519; A61K 39/395; A61P 35/00; C07K 16/28; G01N 33/574

(52) U.S. Cl. CPC C07K 16/32; A61K 31/519; A61K 39/39558; A61P 35/00; C07K 16/2827; G01N 33/57484; A61K 2039/505; C07K 2317/31; C07K 2317/76; G01N 2333/912

(57) ABSTRACT

The present application provides a method of treating tumor: administrating a HER2 inhibitor, and said subject comprises an alteration in a protein HER2, CDK12. The present application provides a medicinal product comprising: the HER2 inhibitor and the multiple CDK inhibitor, showing significant inhibition of tumor cell growth.

This application is a continuation of PCT/CN2022/077313 filed Feb. 22, 2022 and claims the benefit of PCT/CN2021/0077490 filed Feb. 23, 2021.

### BACKGROUND OF THE INVENTION

In accordance with 37 CFR § 1.833-1835 and 37 CFR § 1.77(b)(5), the specification makes reference to a Sequence Listing submitted electronically as a .xml file named “548943US_ST26_102723”. The .xml file was generated on Oct. 27, 2023 and is 163,339 bytes in size. The entire contents of the Sequence Listing are hereby incorporated by reference.

Breast cancer is the most common cancer in women worldwide, of which approximately 15-20% cases are characterized with HER2 (ERBB2) overexpression or amplification and may be treated with HER2-directed targeted agents.

In recent decades, the introduction of HER2-targeted therapies, most notably, trastuzumab, pertuzumab, antibody drug conjugates (ADCs, eg, T-DM1 and DS8201a), and tyrosine kinase inhibitors (TKIs, eg, lapatinib, neratinib, pyrotinib and tucatinib) has shown dramatic improvements in the prognosis of patients with HER2-positive breast cancer.

However, some patients are not responsive to such HER2-targeted therapies, and even show drug resistance after a period of treatment. And the HER2-targeted therapies may lead to some change of the genome of the patients leading the therapy strategy more complicated.

Hence, there is still an urgent medical need to develop the next generation anti-HER2 agents to increase treatment effect and reverse drug resistance.

### SUMMARY OF THE INVENTION

In the present application, it is found that subjects bearing CDK12-amplified tumor, and/or, having an alteration in HER2 protein and/or CDK12 protein, and/or having an alteration in a gene encoding them, are more responsive (for example, more likely to have significant decrease of tumor volume; and/or more likely to have longer PFS (Progression-Free-Survival)) to the anti-HER2 bispecific antibody of the present disclosure.

The present disclosure provides a method of preventing, alleviating or treating tumor or inhibiting tumor growth in a subject, comprising: administrating a HER2 inhibitor of present application to a subject having an alteration in a protein and/or a gene encoding the protein, and the protein comprises HER2 and/or CDK12. The present application also provides a method as well as a system for detecting the alteration in the subject in order to determine whether the HER2 inhibitor of present application is suitable for the subject to be administrated. The present application also provides the method of treating tumor using the HER2 inhibitor of present application, a multiple CDK inhibitor of present application and/or the immune checkpoint inhibitor of present application. Also, the present application provides a medicinal product comprising the HER2 inhibitor of present application, the multiple CDK inhibitor of present application and/or the immune checkpoint inhibitor of present application.

In one aspect, the present application provides a method of preventing, alleviating or treating tumor or inhibiting tumor growth in a subject, comprising: administrating to the subject a HER2 inhibitor, wherein the subject comprises an alteration in a protein and/or a gene encoding the protein, and the protein comprises HER2 and/or CDK12.

In another aspect, the present application provides a method of preventing, alleviating or treating a tumor or inhibiting tumor growth in a subject in need of, comprising: administrating to the subject a HER2 inhibitor, wherein the tumor is CDK12-amplified tumor.

In some embodiments, the HER2 inhibitor is capable of inhibiting human HER2.

In some embodiments, the HER2 inhibitor is a HER2 antibody or an antigen binding portion thereof and/or a conjugate thereof.

In some embodiments, the HER2 inhibitor is selected from a group consisting of Pertuzumab, Trastuzumab and Margetuximab.

In some embodiments, the HER2 inhibitor is selected from a group consisting of DS8201a and T-DM1.

In some embodiments, the HER2 inhibitor is a bispecific antibody or an antigen binding portion thereof, and is capable of binding to different epitopes of human HER2.

In some embodiments, the HER2 inhibitor is a bispecific antibody or the antigen binding portion thereof, and the bispecific antibody or the antigen binding portion thereof has a first light chain and a second light chain, and wherein the first light chain and the second light chain comprises a same amino acid sequence.

In some embodiments, the HER2 inhibitor is a bispecific antibody or the antigen binding portion thereof, and the bispecific antibody or the antigen binding portion thereof has a first heavy chain and a second heavy chain, and wherein the first heavy chain and the second heavy chain are capable of correctly assembling with the light chains respectively under physiological conditions or during in vitro protein expression.

In some embodiments, the first light chain and the second light chain is capable of assembling with a heavy chain of Pertuzumab and a heavy chain of Trastuzumab, respectively.

In some embodiments, the variable region of the first light chain and/or the second light chain comprises an amino acid sequence as set forth in any one of SEQ ID NO: 91-96.

In some embodiments, variable region of the first light chain and/or the second light chain comprises an amino acid sequence as set forth in SEQ ID NO: 91.

In some embodiments, the first light chain and the second light chain is selected from a light chain of Pertuzumab or a mutant thereof, a light chain of Trastuzumab or a mutant thereof, respectively.

In some embodiments, the first light chain comprises an amino acid sequence as set forth in any one of SEQ ID NO: 65-70, and/or, the second light chain comprises an amino acid sequence as set forth in any one of SEQ ID NO: 65-70.

In some embodiments, the heavy chain variable regions are a heavy chain variable region of Pertuzumab and a heavy chain variable region of Trastuzumab, respectively.

In some embodiments, variable region of the first heavy chain comprises an amino acid sequence as set forth in SEQ ID NO: 87; and variable region of the second heavy chain comprises an amino acid sequence as set forth in SEQ ID NO: 88.

In some embodiments, the first heavy chain and the second heavy chain comprises a constant region, and the constant region is originated from human IgG constant region.

In some embodiments, Fc fragment sequences of the heavy chains comprise sequences as set forth in any one of SEQ ID NO: 89-90, 101-104.

In some embodiments, two heavy chains thereof comprise a sequence as set forth in any one of SEQ ID NO: 80-81, 83-84, 97-100.

In some embodiments, the HER2 inhibitor is administrated to the subject at a dose of about 15 mg/kg to about 35 mg/kg.

In some embodiments, the dose of the HER2 inhibitor is about 20 mg/kg to about 30 mg/kg.

In some embodiments, the dose of the HER2 inhibitor is about 20 mg/kg.

In some embodiments, the dose of the HER2 inhibitor is about 30 mg/kg.

In some embodiments, the HER2 inhibitor is administrated about once every two weeks or about once every three weeks.

In some embodiments, the dose of the HER2 inhibitor is about 20 mg/kg, and the HER2 inhibitor is administered once every two weeks.

In some embodiments, the dose of the HER2 inhibitor is about 30 mg/kg, and the HER2 inhibitor is administered once every three weeks.

In some embodiments, the HER2 inhibitor is administrated by intravenous administration.

In some embodiments, the alteration comprises a mutation, an amplification, a fusion and/or a rearrangement in the gene.

In some embodiments, the alteration comprises a mutation and/or an amplification in the protein and/or the mRNA encoding the protein.

In some embodiments, the alteration comprises an amplification of the protein, an amplification of mRNA encoding the protein, and/or an amplification of the gene.

In some embodiments, the alteration comprises at least one mutation of the HER2 protein, wherein the mutation comprises T862A, H878Y and/or R897W.

In some embodiments, the alteration comprises a mutation of the HER2 protein, wherein the mutation comprises T862A, H878Y and R897W.

In some embodiments, the alteration comprises an alteration of CDK12 gene.

In some embodiments, the alteration comprises an amplification of CDK12 gene.

In some embodiments, the alteration comprises a co-amplification of CDK12 gene and HER2 gene.

In some embodiments, the amplification comprises an enhanced DNA copies of the CDK12 gene and/or the HER2 gene.

In some embodiments, the amplification comprises an enhanced mRNA and/or protein expression level of the CDK12 protein and/or the HER2 protein.

In some embodiments, the subject was not responsive to a conventional therapy for HER2-related tumor.

In some embodiments, the conventional therapy for HER2-related tumor comprises administrating HER2-ADC, pyrotinib, neratinib, tucatinib, trastuzumab and/or pertuzumab.

In some embodiments, the conventional therapy for HER2-related tumor comprises administrating docetaxel, capecitabine and/or lapatinib.

In some embodiments, the tumor comprises solid tumor.

In some embodiments, the tumor comprises metastatic tumor, early tumor and/or locally advanced tumor.

In some embodiments, the tumor comprises HER2 positive tumor and/or HER2 low-expression tumor.

In some embodiments, the tumor comprises a breast cancer and/or a gastric cancer.

In some embodiments, the breast cancer comprises HER2 positive breast cancer and/or HER2 low-expression breast cancer.

In some embodiments, the breast cancer comprises early breast cancer, locally advanced breast cancer and/or metastatic breast cancer; and/or the gastric cancer comprises early gastric cancer, locally advanced gastric cancer and/or metastatic gastric cancer.

In some embodiments, the method comprises a following step: detecting the alteration in the subject in order to determine whether the subject is suitable for administrating the HER2 inhibitor.

In some embodiments, the detecting comprising conducting a sequencing of the HER2 protein in the subject.

In some embodiments, the detecting comprising conducting a sequencing of the CDK12 gene.

In some embodiments, the detecting comprising conducting a sequencing of the HER2 gene.

In some embodiments, the sequencing comprises a NGS, and/or a ddPCR.

In some embodiments, the sequencing uses a ctDNA from the subject.

In some embodiments, the sequencing uses peripheral blood and/or tumor tissue from the subject.

In some embodiments, the method further comprises administrating a multiple CDK inhibitor.

In some embodiments, the multiple CDK inhibitor inhibits CDK1, CDK2, CDK5, CDK9 and/or CDK12.

In some embodiments, the multiple CDK inhibitor inhibits CDK12.

In some embodiments, the CDK12 is human CDK12.

In some embodiments, the multiple CDK inhibitor doesn't inhibit CDK4 nor CDK6.

In some embodiments, the multiple CDK inhibitor is selected from a group consisting of: THZ531, Dinaciclib and SR-3029.

In some embodiments, the multiple CDK inhibitor is Dinaciclib.

In some embodiments, the method further comprises administrating an immune checkpoint inhibitor.

In some embodiments, the immune checkpoint inhibitor is capable of specifically binding to PD-L1 and CTLA4.

In some embodiments, the immune checkpoint inhibitor is a bispecific antibody or an antigen binding fragment thereof.

In some embodiments, the immune checkpoint inhibitor is an antigen binding fragment, and the antigen binding fragment comprises Fab, Fab′, F(ab)2, Fv fragment, F(ab′)2, scFv, di-scFv and/or dAb.

In some embodiments, the immune checkpoint inhibitor is a bispecific antibody, and the bispecific antibody is a fully human antibody.

In some embodiments, the immune checkpoint inhibitor is a dimer, and the dimer is formed by two polypeptide chains, with each of the two polypeptide chains comprising an antibody Fc subunit, wherein the dimer comprises two or more immunoglobulin single variable domains (ISVDs), at least one of the ISVDs is specific for PD-L1, and at least one of the ISVDs is specific for CTLA4.

In some embodiments, at least one of the two polypeptide chains comprise both an ISVD specific for PD-L1 and an ISVD specific for CTLA4.

In some embodiments, each of the two polypeptide chains comprises both an ISVD specific for PD-L1 and an ISVD specific for CTLA4.

In some embodiments, for one or both of the two polypeptide chains, the ISVD specific for PD-L1 is fused to the ISVD specific for CTLA4, optionally via a linker.

In some embodiments, for one or both of the two polypeptide chains: the ISVD specific for PD-L1 is fused to the ISVD specific for CTLA4, optionally via a linker; and the ISVD specific for CTLA4 is fused to the antibody Fc subunit, optionally via a linker.

In some embodiments, for one or both of the two polypeptide chains: C terminus of the ISVD specific for PD-L1 is fused to N terminus of the ISVD specific for CTLA4, optionally via a linker, and C terminus of the ISVD specific for CTLA4 is fused to N terminus of the antibody Fc subunit, optionally via a linker.

In some embodiments, for one or both of the two polypeptide chains: the ISVD specific for PD-L1 is fused to the ISVD specific for CTLA4, optionally via a linker; and the ISVD specific for PD-L1 is fused to the antibody Fc subunit, optionally via a linker.

In some embodiments, for one or both of the two polypeptide chains: C terminus of the ISVD specific for CTLA4 is fused to N terminus of the ISVD specific for PD-L1, optionally via a linker; and C terminus of the ISVD specific for PD-L1 is fused to N terminus of the antibody Fc subunit, optionally via a linker.

In some embodiments, the antibody Fc subunit is derived from an IgG Fc subunit.

In some embodiments, the IgG is human IgG1.

In some embodiments, the antibody Fc subunit comprises an amino acid sequence as set forth in any one of SEQ ID NO: 35, 38 and 39.

In some embodiments, the ISVD specific for PD-L1 is capable of binding to N-terminal IgV domain of human PD-L1.

In some embodiments, the ISVD specific for PD-L1 is capable of binding to residues I54, Y56, E58, Q66 and/or R113 of human PD-L1 N-terminal IgV domain, wherein the human PD-L1 N-terminal IgV domain comprises an amino acid sequence as set forth in SEQ ID NO: 64.

In some embodiments, the ISVD specific for PD-L1 is capable of further binding to residues D61, N63, V68, M115, S117, Y123 and/or R125 of human PD-L1 N-terminal IgV domain, wherein the human PD-L1 N-terminal IgV domain comprises an amino acid sequence as set forth in SEQ ID NO: 64.

In some embodiments, the ISVD specific for PD-L1 is capable of binding to a conformational epitope of human PD-L1 N-terminal IgV domain, wherein the conformational epitope comprises residues I54, Y56, E58, Q66 and R113 of the human PD-L1 N-terminal IgV domain, and wherein the human PD-L1 N-terminal IgV domain comprises an amino acid sequence as set forth in SEQ ID NO: 64.

In some embodiments, the ISVD specific for PD-L1 is capable of binding to a conformational epitope of human PD-L1 N-terminal IgV domain, wherein the conformational epitope comprises residues I54, Y56, E58, Q66, R113, D61, N63, V68, M115, S117, Y123 and R125 of the human PD-L1 N-terminal IgV domain, and wherein the human PD-L1 N-terminal IgV domain comprises an amino acid sequence as set forth in SEQ ID NO: 64.

In some embodiments, the ISVD specific for PD-L1 is capable of blocking binding of PD-L1 to PD1.

In some embodiments, the ISVD specific for PD-L1 is capable of blocking binding of PD-L1 to CD80.

In some embodiments, the ISVD specific for PD-L1 cross-competes for binding to PD-L1 with a reference anti-PD-L1 antibody, wherein the reference anti-PD-L1 antibody comprises a heavy chain CDR3 comprising an amino acid sequence as set forth in SEQ ID NO: 1.

In some embodiments, the reference anti-PD-L1 antibody comprises a heavy chain CDR3 comprising an amino acid sequence as set forth in any one of SEQ ID NO: 5 and 9.

In some embodiments, the reference anti-PD-L1 antibody comprises a heavy chain CDR1 comprising an amino acid sequence as set forth in SEQ ID NO: 2.

In some embodiments, the reference anti-PD-L1 antibody comprises a heavy chain CDR1 comprising an amino acid sequence as set forth in any one of SEQ ID NO: 3 and 7.

In some embodiments, the reference anti-PD-L1 antibody comprises a heavy chain CDR2 comprising an amino acid sequence as set forth in any one of SEQ ID NO: 4, 8 and 11.

In some embodiments, the reference anti-PD-L1 antibody is an ISVD specific for PD-L1.

In some embodiments, the reference anti-PD-L1 antibody comprises a heavy chain variable domain comprising an amino acid sequence as set forth in any one of SEQ ID NO: 6, 10, 12, 13, 14 and 15.

In some embodiments, the reference anti-PD-L1 antibody comprises a heavy chain variable domain comprising an amino acid sequence as set forth in SEQ ID NO: 6.

In some embodiments, the ISVD specific for PD-L1 comprises a heavy chain CDR3 comprising an amino acid sequence as set forth in SEQ ID NO: 1.

In some embodiments, the ISVD specific for PD-L1 comprises a heavy chain CDR3 comprising an amino acid sequence as set forth in any one of SEQ ID NO: 5 and 9.

In some embodiments, the ISVD specific for PD-L1 comprises a heavy chain CDR1 comprising an amino acid sequence as set forth in SEQ ID NO: 2.

In some embodiments, the ISVD specific for PD-L1 comprises a heavy chain CDR1 comprising an amino acid sequence as set forth in any one of SEQ ID NO: 3 and 7.

In some embodiments, the ISVD specific for PD-L1 comprises a heavy chain CDR2 comprising an amino acid sequence as set forth in any one of SEQ ID NO: 4, 8 and 11.

In some embodiments, the ISVD specific for PD-L1 comprises a heavy chain variable domain comprising an amino acid sequence as set forth in any one of SEQ ID NO: 6, 10, 12, 13, 14 and 15.

In some embodiments, the ISVD specific for PD-L1 comprises a heavy chain variable domain comprising an amino acid sequence as set forth in SEQ ID NO: 6.

In some embodiments, the ISVD specific for CTLA4 is capable of specifically binding to human CTLA4.

In some embodiments, the ISVD specific for CTLA4 is capable of blocking binding of CTLA4 to CD80.

In some embodiments, the ISVD specific for CTLA4 is capable of blocking binding of CTLA4 to CD86.

In some embodiments, the ISVD specific for CTLA4 cross-competes for binding to CTLA4 with a reference anti-CTLA4 antibody, wherein the reference anti-CTLA4 antibody comprises a heavy chain CDR3 comprising an amino acid sequence as set forth in SEQ ID NO: 19.

In some embodiments, the reference anti-CTLA4 antibody comprises a heavy chain CDR1 comprising an amino acid sequence as set forth in SEQ ID NO: 17.

In some embodiments, the reference anti-CTLA4 antibody comprises a heavy chain CDR2 comprising an amino acid sequence as set forth in SEQ ID NO: 16.

In some embodiments, the reference anti-CTLA4 antibody comprises a heavy chain CDR2 comprising an amino acid sequence as set forth in any one of SEQ ID NO: 18, 21 and 23.

In some embodiments, the reference anti-CTLA4 antibody is an ISVD specific for CTLA4.

In some embodiments, the reference anti-CTLA4 antibody comprises a heavy chain variable domain comprising an amino acid sequence as set forth in any one of SEQ ID NO: 20, 22, and 24-32.

In some embodiments, the reference anti-CTLA4 antibody comprises a heavy chain variable domain comprising an amino acid sequence as set forth in SEQ ID NO: 20.

In some embodiments, the ISVD specific for CTLA4 comprises a heavy chain CDR3 comprising an amino acid sequence as set forth in SEQ ID NO: 19.

In some embodiments, the ISVD specific for CTLA4 comprises a heavy chain CDR1 comprising an amino acid sequence as set forth in SEQ ID NO: 17.

In some embodiments, the ISVD specific for CTLA4 comprises a heavy chain CDR2 comprising an amino acid sequence as set forth in SEQ ID NO: 16.

In some embodiments, the ISVD specific for CTLA4 comprises a heavy chain CDR2 comprising an amino acid sequence as set forth in any one of SEQ ID NO: 18, 21 and 23.

In some embodiments, the ISVD specific for CTLA4 comprises a heavy chain variable domain comprising an amino acid sequence as set forth in any one of SEQ ID NO: 20, 22, and 24-32.

In some embodiments, the ISVD specific for CTLA4 comprises a heavy chain variable domain comprising an amino acid sequence as set forth in SEQ ID NO: 20.

In some embodiments, the dimer is a homodimer.

In some embodiments, the linker comprises an amino acid sequence as set forth in any one of SEQ ID NO: 33-34.

In some embodiments, one or both of the two polypeptide chains comprises an amino acid sequence as set forth in any one of SEQ ID NO: 40-43, 46, 48 and 50.

In some embodiments, one or both of the two polypeptide chains comprises an amino acid sequence as set forth in SEQ ID NO 40.

In some embodiments, the dimer is capable of blocking binding of PD-L1 to PD-1.

In some embodiments, the dimer is capable of blocking binding of PD-L1 to CD80.

In some embodiments, the dimer is capable of blocking binding of CTLA4 to CD80.

In some embodiments, the dimer is capable of blocking binding of CTLA4 to CD86.

In another aspect, the present application provides a medicinal product comprising: a HER2 inhibitor and a multiple CDK inhibitor.

In some embodiments, the HER2 inhibitor is capable of inhibiting human HER2.

In some embodiments, the HER2 inhibitor is a HER2 antibody or an antigen binding portion thereof and/or a conjugate thereof.

In some embodiments, the HER2 inhibitor is selected from a group consisting of Pertuzumab, Trastuzumab and Margetuximab.

In some embodiments, the HER2 inhibitor is selected from a group consisting of DS8201a and T-DM1.

In some embodiments, the HER2 inhibitor is a bispecific antibody or an antigen binding portion thereof, and is capable of binding to different epitopes of human HER2.

In some embodiments, the HER2 inhibitor is a bispecific antibody or the antigen binding portion thereof, and the bispecific antibody or the antigen binding portion thereof has a first light chain and a second light chain, and wherein the first light chain and the second light chain comprises a same amino acid sequence.

In some embodiments, the HER2 inhibitor is a bispecific antibody or the antigen binding portion thereof, and the bispecific antibody or the antigen binding portion thereof has a first heavy chain and a second heavy chain, and wherein the first heavy chain and the second heavy chain are capable of correctly assembling with the light chains respectively under physiological conditions or during in vitro protein expression.

In some embodiments, the first light chain and the second light chain is capable of assembling with a heavy chain of Pertuzumab and a heavy chain of Trastuzumab, respectively.

In some embodiments, variable region of the first light chain and/or the second light chain comprises an amino acid sequence as set forth in any one of SEQ ID NO: 91-96.

In some embodiments, variable region of the first light chain and/or the second light chain comprises an amino acid sequence as set forth in SEQ ID NO: 91.

In some embodiments, the first light chain and the second light chain is selected from a light chain of Pertuzumab or a mutant thereof, a light chain of Trastuzumab or a mutant thereof, respectively.

In some embodiments, the first light chain comprises an amino acid sequence as set forth in any one of SEQ ID NO: 65-70, and/or, the second light chain comprises an amino acid sequence as set forth in any one of SEQ ID NO: 65-70.

In some embodiments, heavy chain variable regions are a heavy chain variable region of Pertuzumab and a heavy chain variable region of Trastuzumab, respectively.

In some embodiments, variable region of the first heavy chain comprises an amino acid sequence as set forth in SEQ ID NO: 87; and variable region of the second heavy chain comprises an amino acid sequence as set forth in SEQ ID NO: 88.

In some embodiments, the first heavy chain and the second heavy chain comprises a constant region, and the constant region is originated from human IgG constant region.

In some embodiments, Fc fragment sequences of the heavy chains comprise sequences as set forth in any one of SEQ ID NO: 89-90, 101-104.

In some embodiments, two heavy chains thereof comprise a sequence as set forth in any one of SEQ ID NO: 80-81, 83-84, 97-100.

In some embodiments, the multiple CDK inhibitor inhibits CDK1, CDK2, CDK5, CDK9 and/or CDK12.

In some embodiments, the multiple CDK inhibitor inhibits CDK12.

In some embodiments, the CDK12 is human CDK12.

In some embodiments, the multiple CDK inhibitor doesn't inhibit CDK4 nor CDK6.

In some embodiments, the multiple CDK inhibitor is selected from a group consisting of: THZ531, Dinaciclib and SR-3029.

In some embodiments, the multiple CDK inhibitor is Dinaciclib.

In some embodiments, the HER2 inhibitor and the multiple CDK inhibitor are not comprised in the same container.

In some embodiments, the HER2 inhibitor, and the multiple CDK inhibitor are comprised in separate containers, respectively.

In some embodiments, the medicinal product further comprises an immune checkpoint inhibitor.

In some embodiments, the immune checkpoint inhibitor is capable of specifically binding to PD-L1 and CTLA4.

In some embodiments, the immune checkpoint inhibitor is a bispecific antibody or an antigen binding fragment thereof.

In some embodiments, the immune checkpoint inhibitor is an antigen binding fragment, and the antigen binding fragment comprises Fab, Fab′, F(ab)2, Fv fragment, F(ab′)2, scFv, di-scFv and/or dAb.

In some embodiments, the immune checkpoint inhibitor is a bispecific antibody, and the bispecific antibody is a fully human antibody.

In some embodiments, the immune checkpoint inhibitor is a dimer, and the dimer is formed by two polypeptide chains, with each of the two polypeptide chains comprising an antibody Fc subunit, wherein the dimer comprises two or more immunoglobulin single variable domains (ISVDs), at least one of the ISVDs is specific for PD-L1, and at least one of the ISVDs is specific for CTLA4.

In some embodiments, at least one of the two polypeptide chains comprise both an ISVD specific for PD-L1 and an ISVD specific for CTLA4.

In some embodiments, each of the two polypeptide chains comprises both an ISVD specific for PD-L1 and an ISVD specific for CTLA4.

In some embodiments, for one or both of the two polypeptide chains, the ISVD specific for PD-L1 is fused to the ISVD specific for CTLA4, optionally via a linker.

In some embodiments, for one or both of the two polypeptide chains: the ISVD specific for PD-L1 is fused to the ISVD specific for CTLA4, optionally via a linker; and the ISVD specific for CTLA4 is fused to the antibody Fc subunit, optionally via a linker.

In some embodiments, for one or both of the two polypeptide chains: C terminus of the ISVD specific for PD-L1 is fused to N terminus of the ISVD specific for CTLA4, optionally via a linker; and C terminus of the ISVD specific for CTLA4 is fused to N terminus of the antibody Fc subunit, optionally via a linker.

In some embodiments, for one or both of the two polypeptide chains: the ISVD specific for PD-L1 is fused to the ISVD specific for CTLA4, optionally via a linker; and the ISVD specific for PD-L1 is fused to the antibody Fc subunit, optionally via a linker.

In some embodiments, for one or both of the two polypeptide chains: C terminus of the ISVD specific for CTLA4 is fused to N terminus of the ISVD specific for PD-L1, optionally via a linker; and C terminus of the ISVD specific for PD-L1 is fused to N terminus of the antibody Fc subunit, optionally via a linker.

In some embodiments, the antibody Fc subunit is derived from an IgG Fc subunit.

In some embodiments, the IgG is human IgG1.

In some embodiments, the antibody Fc subunit comprises an amino acid sequence as set forth in any one of SEQ ID NO: 35, 38 and 39.

In some embodiments, the ISVD specific for PD-L1 is capable of binding to N-terminal IgV domain of human PD-L1.

In some embodiments, the ISVD specific for PD-L1 is capable of binding to residues I54, Y56, E58, Q66 and/or R113 of human PD-L1 N-terminal IgV domain, wherein the human PD-L1 N-terminal IgV domain comprises an amino acid sequence as set forth in SEQ ID NO: 64.

In some embodiments, the ISVD specific for PD-L1 is capable of further binding to residues D61, N63, V68, M115, S117, Y123 and/or R125 of human PD-L1 N-terminal IgV domain, wherein the human PD-L1 N-terminal IgV domain comprises an amino acid sequence as set forth in SEQ ID NO: 64.

In some embodiments, the ISVD specific for PD-L1 is capable of binding to a conformational epitope of human PD-L1 N-terminal IgV domain, wherein the conformational epitope comprises residues I54, Y56, E58, Q66 and R113 of the human PD-L1 N-terminal IgV domain, and wherein the human PD-L1 N-terminal IgV domain comprises an amino acid sequence as set forth in SEQ ID NO: 64.

In some embodiments, the ISVD specific for PD-L1 is capable of binding to a conformational epitope of human PD-L1 N-terminal IgV domain, wherein the conformational epitope comprises residues I54, Y56, E58, Q66, R113, D61, N63, V68, M115, S117, Y123 and R125 of the human PD-L1 N-terminal IgV domain, and wherein the human PD-L1 N-terminal IgV domain comprises an amino acid sequence as set forth in SEQ ID NO: 64.

In some embodiments, the ISVD specific for PD-L1 is capable of blocking binding of PD-L1 to PD1.

In some embodiments, the ISVD specific for PD-L1 is capable of blocking binding of PD-L1 to CD80.

In some embodiments, the ISVD specific for PD-L1 cross-competes for binding to PD-L1 with a reference anti-PD-L1 antibody, wherein the reference anti-PD-L1 antibody comprises a heavy chain CDR3 comprising an amino acid sequence as set forth in SEQ ID NO: 1.

In some embodiments, the reference anti-PD-L1 antibody comprises a heavy chain CDR3 comprising an amino acid sequence as set forth in any one of SEQ ID NO: 5 and 9.

In some embodiments, the reference anti-PD-L1 antibody comprises a heavy chain CDR1 comprising an amino acid sequence as set forth in SEQ ID NO: 2.

In some embodiments, the reference anti-PD-L1 antibody comprises a heavy chain CDR1 comprising an amino acid sequence as set forth in any one of SEQ ID NO: 3 and 7.

In some embodiments, the reference anti-PD-L1 antibody comprises a heavy chain CDR2 comprising an amino acid sequence as set forth in any one of SEQ ID NO: 4, 8 and 11.

In some embodiments, the reference anti-PD-L1 antibody is an ISVD specific for PD-L1.

In some embodiments, the reference anti-PD-L1 antibody comprises a heavy chain variable domain comprising an amino acid sequence as set forth in any one of SEQ ID NO: 6, 10, 12, 13, 14 and 15.

In some embodiments, the reference anti-PD-L1 antibody comprises a heavy chain variable domain comprising an amino acid sequence as set forth in SEQ ID NO: 6.

In some embodiments, the ISVD specific for PD-L1 comprises a heavy chain CDR3 comprising an amino acid sequence as set forth in SEQ ID NO: 1.

In some embodiments, the ISVD specific for PD-L1 comprises a heavy chain CDR3 comprising an amino acid sequence as set forth in any one of SEQ ID NO: 5 and 9.

In some embodiments, the ISVD specific for PD-L1 comprises a heavy chain CDR1 comprising an amino acid sequence as set forth in SEQ ID NO: 2.

In some embodiments, the ISVD specific for PD-L1 comprises a heavy chain CDR1 comprising an amino acid sequence as set forth in any one of SEQ ID NO: 3 and 7.

In some embodiments, the ISVD specific for PD-L1 comprises a heavy chain CDR2 comprising an amino acid sequence as set forth in any one of SEQ ID NO: 4, 8 and 11.

In some embodiments, the ISVD specific for PD-L1 comprises a heavy chain variable domain comprising an amino acid sequence as set forth in any one of SEQ ID NO: 6, 10, 12, 13, 14 and 15.

In some embodiments, the ISVD specific for PD-L1 comprises a heavy chain variable domain comprising an amino acid sequence as set forth in SEQ ID NO: 6.

In some embodiments, the ISVD specific for CTLA4 is capable of specifically binding to human CTLA4.

In some embodiments, the ISVD specific for CTLA4 is capable of blocking binding of CTLA4 to CD80.

In some embodiments, the ISVD specific for CTLA4 is capable of blocking binding of CTLA4 to CD86.

In some embodiments, the ISVD specific for CTLA4 cross-competes for binding to CTLA4 with a reference anti-CTLA4 antibody, wherein the reference anti-CTLA4 antibody comprises a heavy chain CDR3 comprising an amino acid sequence as set forth in SEQ ID NO: 19.

In some embodiments, the reference anti-CTLA4 antibody comprises a heavy chain CDR1 comprising an amino acid sequence as set forth in SEQ ID NO: 17.

In some embodiments, the reference anti-CTLA4 antibody comprises a heavy chain CDR2 comprising an amino acid sequence as set forth in SEQ ID NO: 16.

In some embodiments, the reference anti-CTLA4 antibody comprises a heavy chain CDR2 comprising an amino acid sequence as set forth in any one of SEQ ID NO: 18, 21 and 23.

In some embodiments, the reference anti-CTLA4 antibody is an ISVD specific for CTLA4.

In some embodiments, the reference anti-CTLA4 antibody comprises a heavy chain variable domain comprising an amino acid sequence as set forth in any one of SEQ ID NO: 20, 22, and 24-32.

In some embodiments, the reference anti-CTLA4 antibody comprises a heavy chain variable domain comprising an amino acid sequence as set forth in SEQ ID NO: 20.

In some embodiments, the ISVD specific for CTLA4 comprises a heavy chain CDR3 comprising an amino acid sequence as set forth in SEQ ID NO: 19.

In some embodiments, the ISVD specific for CTLA4 comprises a heavy chain CDR1 comprising an amino acid sequence as set forth in SEQ ID NO: 17.

In some embodiments, the ISVD specific for CTLA4 comprises a heavy chain CDR2 comprising an amino acid sequence as set forth in SEQ ID NO: 16.

In some embodiments, the ISVD specific for CTLA4 comprises a heavy chain CDR2 comprising an amino acid sequence as set forth in any one of SEQ ID NO: 18, 21 and 23.

In some embodiments, the ISVD specific for CTLA4 comprises a heavy chain variable domain comprising an amino acid sequence as set forth in any one of SEQ ID NO: 20, 22, and 24-32.

In some embodiments, the ISVD specific for CTLA4 comprises a heavy chain variable domain comprising an amino acid sequence as set forth in SEQ ID NO: 20.

In some embodiments, the dimer is a homodimer.

In some embodiments, the linker comprises an amino acid sequence as set forth in any one of SEQ ID NO: 33-34.

In some embodiments, one or both of the two polypeptide chains comprises an amino acid sequence as set forth in any one of SEQ ID NO: 40-43, 46, 48 and 50.

In some embodiments, one or both of the two polypeptide chains comprises an amino acid sequence as set forth in SEQ ID NO 40.

In some embodiments, the dimer is capable of blocking binding of PD-L1 to PD-1.

In some embodiments, the dimer is capable of blocking binding of PD-L1 to CD80.

In some embodiments, the dimer is capable of blocking binding of CTLA4 to CD80.

In some embodiments, the dimer is capable of blocking binding of CTLA4 to CD86.

In some embodiments, the medicinal product is a pharmaceutical composition.

In some embodiments, the HER2 inhibitor, the immune checkpoint inhibitor, and the multiple CDK inhibitor are not comprised in the same container.

In some embodiments, aid HER2 inhibitor, the immune checkpoint inhibitor, and the multiple CDK inhibitor are comprised in separate containers, respectively.

In another aspect, the present application provides a use of a HER2 inhibitor in combination with a multiple CDK inhibitor and/or an immune checkpoint inhibitor in the preparation of a medicament for alleviating or treating tumor or inhibiting tumor growth in a subject, wherein the HER2 inhibitor is as defined in the present application, the multiple CDK inhibitor is as defined in the present application and the immune checkpoint inhibitor is as defined in the present application.

In another aspect, the present application provides a use of the medicinal product as defined in the present application in the preparation of a medicament for alleviating or treating tumor or inhibiting tumor growth in a subject.

In another aspect, the present application provides a method of determining whether a subject is suitable for administrating a HER2 inhibitor, comprising: detecting an alteration in the subject, if the alteration exists, the subject is suitable for administrating the HER2 inhibitor, wherein the alteration is in a protein and/or a gene encoding the protein, and the protein comprises HER2 and/or CDK12.

In some embodiments, the HER2 inhibitor is capable of inhibiting human HER2.

In some embodiments, the HER2 inhibitor is a HER2 antibody or an antigen binding portion thereof and/or a conjugate thereof.

In some embodiments, the HER2 inhibitor is selected from a group consisting of Pertuzumab, Trastuzumab and Margetuximab.

In some embodiments, the HER2 inhibitor is selected from a group consisting of DS8201a and T-DM1.

In some embodiments, the HER2 inhibitor is a bispecific antibody or an antigen binding portion thereof, and is capable of binding to different epitopes of human HER2.

In some embodiments, the HER2 inhibitor is a bispecific antibody or the antigen binding portion thereof, and the bispecific antibody or the antigen binding portion thereof has a first light chain and a second light chain, and wherein the first light chain and the second light chain comprises a same amino acid sequence.

In some embodiments, the HER2 inhibitor is a bispecific antibody or the antigen binding portion thereof, and the bispecific antibody or the antigen binding portion thereof has a first heavy chain and a second heavy chain, and wherein the first heavy chain and the second heavy chain are capable of correctly assembling with the light chains respectively under physiological conditions or during in vitro protein expression.

In some embodiments, the first light chain and the second light chain is capable of assembling with a heavy chain of Pertuzumab and a heavy chain of Trastuzumab, respectively.

In some embodiments, variable region of the first light chain and/or the second light chain comprises an amino acid sequence as set forth in any one of SEQ ID NO: 91-96.

In some embodiments, variable region of the first light chain and/or the second light chain comprises an amino acid sequence as set forth in SEQ ID NO: 91.

In some embodiments, the first light chain and the second light chain is selected from a light chain of Pertuzumab or a mutant thereof, a light chain of Trastuzumab or a mutant thereof, respectively.

In some embodiments, the first light chain comprises an amino acid sequence as set forth in any one of SEQ ID NO: 65-70, and/or, the second light chain comprises an amino acid sequence as set forth in any one of SEQ ID NO: 65-70.

In some embodiments, the heavy chain variable regions are a heavy chain variable region of Pertuzumab and a heavy chain variable region of Trastuzumab, respectively.

In some embodiments, the variable region of the first heavy chain comprises an amino acid sequence as set forth in SEQ ID NO: 87; and variable region of the second heavy chain comprises an amino acid sequence as set forth in SEQ ID NO: 88.

In some embodiments, the first heavy chain and the second heavy chain comprises a constant region, and the constant region is originated from human IgG constant region.

In some embodiments, Fc fragment sequences of the heavy chains comprise sequences as set forth in any one of SEQ ID NO: 89-90, 101-104.

In some embodiments, two heavy chains thereof comprise a sequence as set forth in any one of SEQ ID NO: 80-81, 83-84, 97-100.

In some embodiments, the alteration comprises a mutation, an amplification, a fusion and/or a rearrangement in the gene.

In some embodiments, the alteration comprises a mutation and/or an amplification in the protein and/or the mRNA encoding the protein.

In some embodiments, the alteration comprises an amplification of the protein, an amplification of mRNA encoding the protein, and/or an amplification of the gene.

In some embodiments, the alteration comprises at least one mutation of the HER2 protein, wherein the mutation comprises T862A, H878Y and/or R897W.

In some embodiments, the alteration comprises a mutation of the HER2 protein, wherein the mutation comprises T862A, H878Y and R897W.

In some embodiments, the alteration comprises an alteration of CDK12 gene.

In some embodiments, the alteration comprises an amplification of CDK12 gene.

In some embodiments, the alteration comprises a co-amplification of CDK12 gene and HER2 gene.

In some embodiments, the amplification comprises an enhanced DNA copy of the CDK12 gene and/or the HER2 gene.

In some embodiments, the amplification comprises an enhanced mRNA and/or protein expression level of the CDK12 protein and/or the HER2 protein.

In some embodiments, the detecting comprising conducting a sequencing of the HER2 protein in the subject.

In some embodiments, the detecting comprising conducting a sequencing of the CDK12 gene.

In some embodiments, the detecting comprising conducting a sequencing of the HER2 gene.

In some embodiments, the sequencing comprises a NGS, and/or a ddPCR.

In some embodiments, the sequencing uses a ctDNA from the subject.

In some embodiments, the sequencing uses peripheral blood and/or tumor tissue from the subject.

In another aspect, the present application provides a system for determining whether a subject is suitable for administrating a HER2 inhibitor, comprising, a detection module configured to detect an alteration in a protein and/or a gene encoding the protein in the subject, wherein the protein comprises HER2 and/or CDK12.

In some embodiments, the alteration comprises a mutation, an amplification, a fusion and/or a rearrangement in the gene.

In some embodiments, the alteration comprises a mutation and/or an amplification in the protein and/or the mRNA encoding the protein.

In some embodiments, the alteration comprises an amplification of the protein, an amplification of mRNA encoding the protein, and/or an amplification of the gene.

In some embodiments, the alteration comprises at least one mutation of the HER2 protein, wherein the mutation comprises T862A, H878Y and/or R897W.

In some embodiments, the alteration comprises a mutation of the HER2 protein, wherein the mutation comprises T862A, H878Y and R897W.

In some embodiments, the alteration comprises an alteration of CDK12 gene.

In some embodiments, the alteration comprises an amplification of CDK12 gene.

In some embodiments, the alteration comprises a co-amplification of CDK12 gene and HER2 gene.

In some embodiments, the amplification comprises an enhanced DNA copies of the CDK12 gene and/or the HER2 gene.

In some embodiments, the amplification comprises an enhanced mRNA and/or protein expression level of the CDK12 protein and/or the HER2 protein.

In some embodiments, the detecting module is configured to conduct a sequencing of the HER2 protein.

In some embodiments, the detecting module comprises an agent for sequencing of the HER2 protein.

In some embodiments, the detecting module is configured to conduct a sequencing of the CDK12 gene.

In some embodiments, the detecting module is configured to conduct a sequencing of the HER2 gene.

In some embodiments, the detecting module comprises an agent for sequencing of the CDK12 gene and/or an agent for sequencing the HER2 gene.

In some embodiments, the system comprises a sample collecting module.

In some embodiments, the sample collecting module is configured to collect a ctDNA from the subject.

In some embodiments, the sample collecting module collects peripheral blood and/or tumor tissue from the subject.

In some embodiments, the sample collecting module comprises an agent for collecting and/or an agent for isolating ctDNA and/or for isolating tumor tissue DNA.

In some embodiments, the HER2 inhibitor is capable of inhibiting human HER2.

In some embodiments, the HER2 inhibitor is a HER2 antibody or an antigen binding portion thereof and/or a conjugate thereof.

In some embodiments, the HER2 inhibitor is selected from a group consisting of Pertuzumab, Trastuzumab and Margetuximab.

In some embodiments, the HER2 inhibitor is selected from a group consisting of DS8201a and T-DM1.

In some embodiments, the HER2 inhibitor is a bispecific antibody or an antigen binding portion thereof, and is capable of binding to different epitopes of human HER2.

In some embodiments, the HER2 inhibitor is a bispecific antibody or the antigen binding portion thereof, and the bispecific antibody or the antigen binding portion thereof has a first light chain and a second light chain, and wherein the first light chain and the second light chain comprises a same amino acid sequence.

In some embodiments, the HER2 inhibitor is a bispecific antibody or the antigen binding portion thereof, and the bispecific antibody or the antigen binding portion thereof has a first heavy chain and a second heavy chain, and wherein the first heavy chain and the second heavy chain are capable of correctly assembling with the light chains respectively under physiological conditions or during in vitro protein expression.

In some embodiments, the first light chain and the second light chain is capable of assembling with a heavy chain of Pertuzumab and a heavy chain of Trastuzumab, respectively.

In some embodiments, variable region of the first light chain and/or the second light chain comprises an amino acid sequence as set forth in any one of SEQ ID NO: 91-96.

In some embodiments, variable region of the first light chain and/or the second light chain comprises an amino acid sequence as set forth in SEQ ID NO: 91.

In some embodiments, the first light chain and the second light chain is selected from a light chain of Pertuzumab or a mutant thereof, a light chain of Trastuzumab or a mutant thereof, respectively.

In some embodiments, the first light chain comprises an amino acid sequence as set forth in any one of SEQ ID NO: 65-70, and/or, the second light chain comprises an amino acid sequence as set forth in any one of SEQ ID NO: 65-70.

In some embodiments, heavy chain variable regions are a heavy chain variable region of Pertuzumab and a heavy chain variable region of Trastuzumab, respectively.

In some embodiments, variable region of the first heavy chain comprises an amino acid sequence as set forth in SEQ ID NO: 87; and variable region of the second heavy chain comprises an amino acid sequence as set forth in SEQ ID NO: 88.

In some embodiments, the first heavy chain and the second heavy chain comprises a constant region, and the constant region is originated from human IgG constant region.

In some embodiments, Fc fragment sequences of the heavy chains comprise sequences as set forth in any one of SEQ ID NO: 89-90, 101-104.

In some embodiments, two heavy chains thereof comprise a sequence as set forth in any one of SEQ ID NO: 80-81, 83-84, 97-100.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates NGS information from tissue ctDNA of 22 patients.

FIG. 2 illustrates NGS information from peripheral blood ctDNA of 22 patients.

FIG. 3 illustrates the HER2 positive breast tumor cells BT474 are inhibited by the HER2 inhibitor of the present application and the multiple CDK inhibitor of the present application.

FIG. 4 illustrates the HER2 positive gastric tumor cells N87 are inhibited by the HER2 inhibitor of the present application and the multiple CDK inhibitor of the present application.

FIGS. 5-6 illustrates the CDK12-amplified solid tumor cells are inhibited by the HER2 inhibitor of the present application and the multiple CDK inhibitor of the present application.

FIGS. 7-8 illustrates the CDK12-amplified solid tumor cells are inhibited by the HER2 inhibitor of the present application and the multiple CDK inhibitor of the present application.

FIG. 9 illustrates the HER2 positive gastric tumor cells N87 are significantly inhibited by the HER2 inhibitor of the present application.

FIGS. 10-11 illustrate the CDK12-amplified solid tumor cells are inhibited by the HER2 inhibitor of the present application.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “HER2”, as used herein, generally refers to the type I transmembrane protein, also known as c-erbB2, ErbB2 or Neu, belonging to the family of epidermal growth factor receptors. In the context of the present application, the term “HER2” also encompasses homologues, variants and isoforms, including splice isoforms, of HER2. The term “HER2” further encompasses proteins having the sequence of one or more of a HER2 homologue, variant and isoform, as well as fragments of the sequences, provided that the variant proteins (including isoforms), homologous proteins and/or fragments are recognized by one or more HER2 specific antibodies, such as provided as Pertuzumab, Trastuzumab and Margetuximab. The HER2 may be a human HER2. The human HER2 gene is mapped to chromosomal location 17q12, and the genomic sequence of HER2 gene can be found in GenBank at NG_007503.1. In human, there are five HER2 isoforms: A, B, C, D, and E; the term “HER2” is used herein to refer collectively to all HER2 isoforms.

The term “CDK”, as used herein, generally refers to a cyclin-dependent kinase. A CDK may bind a cyclin (e.g., Cyclin H), which is a regulatory protein. CDKs may phosphorylate their substrates at serines and threonines. CDK may comprise CDK1, CDK2, CDK2, CDK4, CDK5, CDK7, CDK8, CDK9, CDK10, CDK11, CDK12, CDK13, CDK14, CDK16, CDK18 and CDK20.

The term CDK inhibitor may refer to selective transcriptional CDK inhibitor. For example, the CDK inhibitor may be useful for the inhibition of selective transcriptional CDKs, particularly selective transcriptional CDK1, CDK2, CDK5, CDK9 and/or CDK12, more particularly selective transcriptional CDK12.

The term “CDK12”, as used herein, generally refers to cyclin-dependent kinase 12, which is a key regulator of the cell cycle. CDK12 can also be called CRK7; CRKR or CRKRS. CDK12 is located on chromosome 17, within the 17q21 locus that contains several candidate genes for breast cancer susceptibility (Kauraniemi et al, Cancer Res., 2001, 61(22), 8235-8240). The gene sequence of human CDK12 gene can be found in GenBank at 51755. The CDK12 gene is conserved in chimpanzee, Rhesus monkey, dog, cow, mouse, rat, chicken, and frog.

The term “CTLA4”, as used herein, generally refers to Cytotoxic T-Lymphocyte-Associated protein 4, its functional variant and/or its functional fragments. CTLA4 is an immunoinhibitory receptor belonging to the CD28 family. CTLA4 is expressed exclusively on T cells (CD 4⁺ and CD 8⁺ cells) in vivo, and binds to two ligands, CD80 and CD86 (also called B7-1 and B7-2, respectively). For example, the term “CTLA4” may comprise a polypeptide or a fragment thereof having at least about 85% amino acid sequence identity to NCBI Accession No. AAL07473.1 and that specifically binds to CD80 and/or CD86. The term “CTLA4” may comprise the entire CTLA4 receptor, its extracellular domain, and fusion proteins comprising a functionally active portion of CTLA4 covalently linked to a second moiety, e.g., a protein domain. The term “CTLA4” may comprise variants which vary in amino acid sequence from naturally occurring CTLA4 but which retain the ability to specifically bind to the ligand CD80 and/or CD86. The term “CTLA4” as used herein may comprise human CTLA4 (hCTLA4), variants, isoforms, and species homologs of hCTLA4, and analogs having at least one common epitope with hCTLA4. For example, the term “CTLA4” may also encompass CTLA4 from other species, such as other mammals, for example, rat, mouse, rabbit, non-human primate, pig, or bovine. The complete hCTLA4 sequence can be found under GenBank Accession No. 1493.

The term “PD-L1”, as used herein, generally refers to the Programmed Death Ligand 1 protein, its functional variant and/or its functional fragments. PD-L1 is also known as cluster of differentiation 274 (CD274) or B7 homolog 1 (B7-HI), and is a protein encoded by the CD274 gene (in human). PD-L1 binds to its receptor, programmed cell death protein 1 (PD-1), which is expressed in activated T cells, B cells, and macrophages (Ishida et al., 1992 EMBO J. 11:3887-3395; Okazaki et al., Autoimmune dilated cardiomyopathy in PD-1 receptor-deficient mice. Science, 2001; 291: 319-22). The complexation of PD-L1 and PD-1 exerts immunosuppressive effects by inhibiting T cell proliferation and cytokine production of IL-2 and IFN-γ (Freeman et al., Engagement of PD-1 immunoinhibitory receptor by a novel B7 family member leads to negative regulation of lymphocyte activation, J. Exp. Med. 2000, 192:1027-1034; Carter et al., PD-1: PD-L inhibitory pathway affects both CD4(+) and CD8(+) T cells and is overcome by IL-2. Eur. J. Immunol. 2002, 32:634-643). For example, the term “PD-L1” may comprise a polypeptide or a fragment thereof having at least about 85% amino acid sequence identity to NCBI Accession No. Q9NZQ7 and that specifically binds PD1. The term PD-L1 includes the entire PD-L1 ligand, soluble PD-L1 ligand, and fusion proteins comprising a functionally active portion of PD-L1 ligand covalently linked to a second moiety, e.g., a protein domain. Also included within the definition of PD-L1 are variants which vary in amino acid sequence from naturally occurring PD-L1 but which retain the ability to specifically bind to the receptor PD1. Further included within the definition of PD-L1 are variants which enhance the biological activity of PD1. PD-L1 sequences are known in the art and are provided, for example, at GenBank Accession Numbers 29126. The term “PD-L1” as used herein includes human PD-L1 (hPD-L1), variants, isoforms, and species homologs of hPD-L1, and analogs having at least one common epitope with hPD-L1. For example, the term “PD-L1” also encompasses PD-L1 from other species, such as other mammals, for example, rat, mouse, rabbit, non-human primate, pig, or bovine. The complete hPD-L1 sequence can be found under GenBank Accession No. 29126.

The term “antibody”, as used herein, generally refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, poly-specific, non-specific, humanized, single-chain, chimeric, synthetic, recombinant, hybrid, mutated, and grafted antibodies. The term “antibody” may also comprise antibody fragments such as Fab, F(ab′₂, Fv, scFv, Fd, dAb, and other antibody fragments that retain antigen-binding function, i.e., the ability to bind, for example, HER2 specifically. Typically, such fragments would comprise an antigen-binding domain.

The term “bispecific antibody”, as used herein, generally refers to an antibody that can respectively bind with two different antigens or the antigen epitopes thereof. For example, the bispecific antibody may comprise at least one kind of light chain or the fragment thereof, as well as at least one kind of heavy chain or the fragment thereof. For example, the bispecific antibody may comprise one light chain or the fragment thereof, which may specifically bind to both a first antigen or the antigen epitope thereof and a second antigen or the antigen epitope thereof. For example, the bispecific antibody may comprise two heavy chains or the fragment thereof, which bind to the first antigen or the antigen epitope thereof and the second antigen or the antigen epitope thereof, respectively. For example, the first antigen or the antigen epitope thereof and the second antigen or the antigen epitope thereof may be two different HER2 antigen.

The term “HER2-positive”, as used herein, generally refers to a solid tumor comprising cells which have HER2 protein present at their cell surface. HER2 protein may be overexpressed, e.g., by gene amplification, the solid tumor overexpressing HER2 may be rated by immunohistochemical scores according to the number of copies of HER2 molecules expressed per cell, and can been determined biochemically (refers to Hudziak et al., Proc. Natl. Acad. Sci. USA 84: 7159-7163 [1987]). For example, the HER2-positive solid tumor may comprise a HER-2 positive breast cancer, the HER-2 positive breast cancer may test positive for estrogen receptor and may be a HER2 nonamplified invasive breast cancer, the HER2-positive breast cancer may be advanced, the HER2-positive breast cancer may be metastatic.

The term “HER2 low-expression”, as used herein, generally refers a solid tumor comprising cells which expresses very low level of HER2. HER2 low-expression may refer to HER2-negative tumors that test IHC 1⁺ or 2⁺ and FISH⁻. The expression level of HER2 may be measured by immunohistochemistry or FISH. For example, the group with low levels of HER2 may be more likely to be of higher grade, EGFR-positive and ER/HER3/HER4-negative.

The term “alteration”, as used herein, generally refers to any change in a protein and/or a gene encoding the protein thereof, the alteration may comprise an increase (or decrease) in the expression level of a protein and/or a gene encoding the protein thereof the alteration may comprise any mutation (such as insertion, substitution and/or deletion) in the sequence of a protein and/or a gene encoding the protein thereof.

The term “amplification”, as used herein, generally refers to the presence of a higher-than-normal number of copies of a genomic nucleic acid sequence. For example, a gene has a higher DNA expression level and/or copies compared to that of in a normal cell. For example, the mRNA and/or protein encoded by the gene may have a higher expression level and/or copies compared to that of in a normal cell. For example, the amplification may occur in a tumor cell and/or a subject suffering the tumor.

The term “co-amplification”, as used herein, generally refers to the process of substantially simultaneous amplification of different genes in a cell. For example, the co-amplification may happen among at least two genes in a tumor cell. For example, the co-amplification may refer to a higher DNA and/or RNA expression level of one gene (e.g. HER2 gene) is accompanied by a higher DNA and/or RNA expression level of another gene (e.g. CDK12 gene). For example, the co-amplification may refer to a higher expression level of one protein (e.g. HER2) is accompanied by a higher expression level of another protein (e.g. CDK12), the co-amplification may happen naturally in a tumor cell.

The term “mutation”, as used herein, generally refers to an alteration in the sequence of a nucleic acid sequence, or an alteration in the sequence of a peptide. For example, the mutation may be a point mutation such as transposition or transversion. For example, the mutation may be deletions, insertions, or duplications. For example, the mutation may comprise a substitution of one amino acid to another amino acid in an amino acid sequence. Such substitution may be illustrated in a format as ANo. (X) B, which means the amino acid A is replaced by amino acid B at the No. (X) amino acid from the N terminal of the amino acid sequence. For example, the mutation may occur in a tumor cell and/or a subject suffering the tumor.

The term “fusion” as used herein, generally refers to a hybrid gene formed from at least two previously independent genes. The fusion may be occurred as a result of translocation, interstitial deletion, or chromosomal inversion. The fusion may be detected by next generation sequencing technology and/or Transcriptome Viewer (TViewer). For example, the fusion may occur in a tumor cell and/or a subject suffering the tumor.

The term “rearrangement”, as used herein, generally refers to a frequent recurring genetic alteration in tumor cell. For example, the rearrangement of a gene may undergo a gene fusion. For example, the rearrangement may be a somatic recombination. For example, the rearrangement may occur in a tumor cell and/or a subject suffering the tumor.

The term “solid tumor”, as used herein, generally refers to an abnormal mass of tissue that usually does not contain liquid areas, the solid tumor may be malignant, and may belong to cancer. Different types of solid tumors are named for the type of cells that form them. For example, the solid tumor may comprise breast cancer.

The term “metastatic”, as used herein, generally refers to a tumor that spreads from its site of origin to another part of the body. For many types of tumor, it may be also called stage IV (4) tumor. The metastatic tumor may develop when the tumor cells break away from the main tumor and enter the bloodstream or lymphatic system. For example, breast cancer that spreads to the lung may be called metastatic breast cancer.

The term “early tumor”, as used herein, generally refers to a tumor that has not grown deeply into nearby tissues. The early tumor may be called early-stage cancer, and/or may be called stage 1 (1) tumor. The early tumor may have not been spread to distant regions.

The term “locally advanced tumor”, as used herein, generally refers to a tumor having grown outside the body part it started in but has not yet spread to other parts of the body. For example, locally advanced breast cancer may be a subset of breast cancer characterized by the most advanced breast tumors in the absence of distant metastasis.

The term “treatment” or “treating”, as used herein, generally refers to clinical intervention in an attempt to alter the natural course of the individual being treated, and may be performed either for prophylaxis or during the course of clinical pathology. Desirable effects of treatment may also comprise preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. For example, the anti-HER2 bispecific antibody may be used to delay development of a disease or to slow the progression of a disease.

The term “preventing”, as used herein, generally refers to delaying the onset, hindering the progress, hindering the appearance, protection against, inhibiting or eliminating the emergence, or reducing the incidence, of such damages, effects or symptoms of a disease or disorder.

The term “alleviating”, as used herein, generally refers to a process by which the severity of a sign or symptom of a disorder is decreased, the alleviating may comprise alleviating but not eliminating a sign or symptom of a disease or disorder.

The term “subject”, as used herein, generally refers to an animal, for example, a human. For example, the subject may comprise “non-human animals”, which may comprise mammals such as rats, mice, rabbits, sheep, cats, dogs, cows, pigs, and non-human primates.

The term “conventional therapy for HER2-related tumor”, as used herein, generally refers to administrating any substances or drugs which block the growth of HER2-related tumor. The conventional therapy for HER2-related tumor may interfere the function of specific molecules responsible for HER2-related (for example, HER2-positive and/or HER2 low expression) tumor cell proliferation and survival. The conventional therapy for HER2-related tumor may comprise any approved drugs specific for treating HER2-related tumor (for example, the HER2-related tumor may be a solid tumor, for example, the HER2-related tumor may at any stage). The conventional therapy for HER2-related tumor may comprise a first-line and/or a second-line approved drug for treating HER2-related tumor (for example, may have been approved for treating a HER2-positive breast cancer). The conventional therapy for HER2-related tumor may comprise any approved drugs suitable for treating HER2-related tumor, including the drugs for universal tumor treatment, for example, a chemotherapy.

The term “failure”, as used herein, generally refers to a subject in need thereof may not respond to a treatment, the failure may comprise there is no significant decrease of a tumor volume, no significant increase of a Progression-free survival (PFS), and/or no biological response after administration a treatment, the failure may indicate that for the specific subject, the former administrated treatment is unsuitable.

The term “HER2-ADC”, as used herein, generally refers to a HER2 targeting antibody drug conjugate and is capable of binding to HER2 on the surface of tumor cell. For example, the HER2-ADC may comprise a trastuzumab emtansine (T-DM1), which may be indicated for treatment of HER2-positive metastatic breast cancer. For example, the HER2-ADC may comprise a Trastuzumab deruxtecan (DS-8201a), which may be indicated for treatment of adult patients with unresectable or metastatic HER2-positive breast cancer. For example, the HER2-ADC may comprise a SYD985, in which trastuzumab is linked to the duocarmycin prodrug seco-duocarmycin-hydroxybenzamide-azaindole orseco-DUBA via a cleavable linker.

The term “DS8201a”, as used herein, generally refers to a HER2-targeted antibody-drug conjugate with a humanized HER2 antibody, cleavable peptide-based linker, and topoisomerase I inhibitor payload. DS8201a may be also named as Trastuzumab deruxtecan.

The term “T-DM1”, or “Kadcyla” as used herein, generally refers to an antibody-drug conjugate trastuzumab emtansine, the T-DM1 has been approved by FDA to treat HER2-positive metastatic breast cancer which has previously been treated with Herceptin (chemical name: trastuzumab) and taxane chemotherapy.

The term “pyrotinib”, as used herein, generally refers an irreversible dual pan-ErbB receptor tyrosine kinase inhibitor. The pyrotinib may target EGFR, HER2, and HER4. The pyrotinib may be used for the treatment of HER2-positive advanced solid tumours. Pyrotinib Racemate is the racemate of Pyrotinib, and Pyrotinib Racemate is a compound having the following formula:

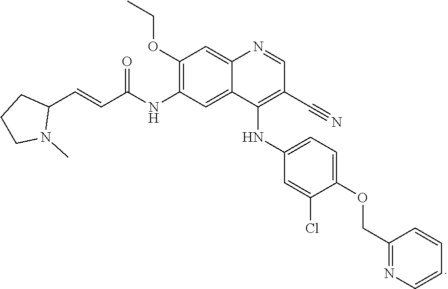 [SMILES: CCOc1cc2ncc(C#N)c(Nc3ccc(OCc4ccccn4)c(Cl)c3)c2cc1NC(=O)/C=C/C1CCCN1C]

The term “neratinib”, as used herein, generally refers a tyrosine kinase inhibitor. Neratinib may be used for extended adjuvant treatment of adults with early stage hormone receptor positive HER2-overexpressed/amplified breast cancer. Neratinib is a compound having the following formula:

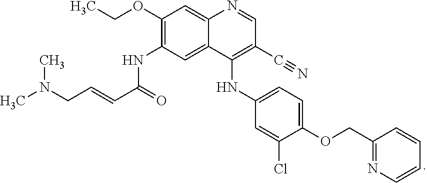 [SMILES: CCOc1cc2ncc(C#N)c(Nc3ccc(OCc4ccccn4)c(Cl)c3)c2cc1NC(=O)/C=C/CN(C)C]

The term “tucatinib”, as used herein, generally refers a small molecule inhibitor of HER2. Tucatinib may be used for advanced unresectable or metastatic HER2-positive breast cancer. Tucatinib is a compound having the following formula:

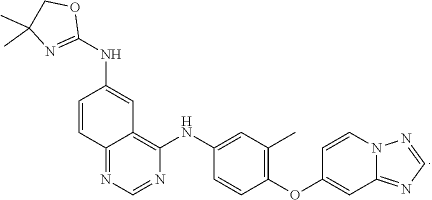 [SMILES: Cc1cc(Nc2ncnc3ccc(NC4=NC(C)(C)CO4)cc23)ccc1Oc1ccn2ncnc2c1]

The term “pertuzumab”, as used herein, generally refers to a monoclonal antibody used for treating HER2-positive breast cancer. The amino acid sequences of the variable light and variable heavy chains of the pertuzumab (OMNITARG®) may be referred to WO2006033700A2.

The term “trastuzumab”, as used herein, generally refers to a monoclonal antibody that interferes with the HER2/neu receptor (tradenames Herclon, Herceptin) (Hudis, 2007, N. Engl. J. Med. 3577(1):39-51).

The term “docetaxel”, as used herein, generally refers to the active ingredient of TAXOTERE® or else TAXOTERE® itself. Docetaxel is a compound having the following formula:

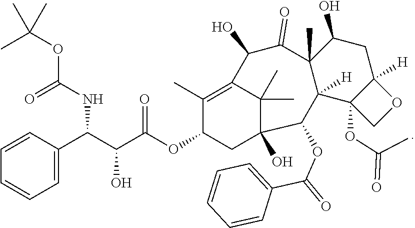

The term “capecitabine”, as used herein, generally refers to a chemotherapeutic agent that is a prodrug that is converted into 5-FU in the tissues. The chemical name of the capecitabine is pentyl [1-(3,4-dihydroxy-5-methyltetrahydrofuran-2-yl)-5-fluoro-2-oxo-1H-pyrimidin-4-yl]carbamates.

The term “lapatinib”, as used herein, generally refers to an orally active drug for breast cancer and other solid tumors. It is a dual tyrosine kinase inhibitor which interrupts the HER2/neu and epidermal growth factor receptor (EGFR) pathways. It acts as a dual reversible TKI for both these receptors, thus blocking the downstream MAPK/Erk1/2 and PI3K/AKT pathways. Lapatinib is a compound having the following formula:

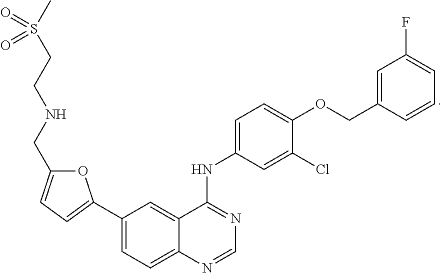 [SMILES: CS(=O)(=O)CCNCc1ccc(-c2ccc3ncnc(Nc4ccc(OCc5cccc(F)c5)c(Cl)c4)c3c2)o1]

The term “Margetuximab”, as used herein, generally refers to a Fc engineered HER2-directed monoclonal antibody, the Margetuximab may be used in combination with chemotherapy, as treatment of adult patients with metastatic HER2-positive breast cancer who have received two or more prior anti-HER2 regimens.

The term “detecting module”, as used herein, generally refers to a unit comprising a hardware and/or a software for detecting the alteration from a sample. For example, the detecting module may be capable of conducting sequencing of an amino acid sequence and/or a nucleic acid.

The term “sequencing”, as used herein, generally refers to any technique known in the art that allows the order of at least some consecutive nucleotides in at least part of a nucleic acid to be identified; and/or at least some consecutive amino acid in at least part of amino acid sequence to be identified. Exemplary sequencing techniques may comprise targeted sequencing, single molecule real-time sequencing, electron microscopy-based sequencing, transistor-mediated sequencing, direct sequencing, random shotgun sequencing, Sanger dideoxy termination sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD® sequencing, MS-PET sequencing, mass spectrometry, and a combination thereof. In some embodiments, sequencing comprises an detecting the sequencing product using an instrument. For example, an ABI PRISM® 377 DNA Sequencer, an ABI PRISM® 310, 3100, 3100-Avant, 3730, or 3730xI Genetic Analyzer, an ABI PRISM® 3700 DNA Analyzer, or an Applied Biosystems SOLiD™ System (all from Applied Biosystems), a Genome Sequencer 20 System (Roche Applied Science), or a mass spectrometer. For example, the sequencing may comprise a high throughput sequencing technique, such as massively parallel signature sequencing (MPSS).

The term “sample collecting module”, as used herein, generally refers to a unit comprising a hardware and/or a software for collecting a sample from a subject in need thereof. For example, the sample collecting module may be used for collecting ctDNA from a subject suffering a HER2-positive solid tumor.

The term “sample”, as used herein, generally refers to any material obtained from the subject in need thereof in order to sequence an amino acid sequence of a specific protein, and/or in order to sequence a nucleoid acid sequence of a specific gene. For example, the sample may comprise cells, organisms, lysed cells, cellular extracts, nuclear extracts, or components of cells or organisms and/or extracellular fluid.

The term “ctDNA”, as used herein, generally refers to circulating tumor DNA, which is free DNA originating from tumor, the ctDNA may suspends in blood of a subject. It is known that among DNA existing in blood only an extremely small amount of ctDNA generally exists as compared with normal cell DNA.

The term “medicinal product”, as used herein, generally refers to any constituent of a therapeutic substance, and the medicinal product may have the same meaning of the terms “medicine,” “medicament,” “therapeutic intervention,” or “pharmaceutical product.” the medicinal product may comprise constituents regardless of their state of matter (e.g., solid, liquid or gas), the medicinal product may comprise multiple constituents that can be included in a therapeutic substance in a mixed state, in an unmixed state and/or in a partially mixed state, the medicinal product may comprise both the active constituents (for example, the anti-HER2 bispecific antibody) and inert constituents of a therapeutic substance. Accordingly, as used herein, the medicinal product may comprise non-active constituents such as, water, colorant or the like.

The term “CDK12 inhibitor”, as used herein, generally refers to any agent which inhibits the activity of CDK12 proteins and/or CDK12/cyclin kinase complexes. The compound or agent may inhibit CDK12 activity by direct or indirect interaction with a CDK12 protein or it may activity act to prevent expression of CDK12 genes. For example, the CDK12 inhibitor may inhibits the activity of CDK12 protein and/or the expression level of CDK12 protein and the mRNA encoded by CDK12 gene.

The term “CDK12”, as used herein, generally refers to a transcription-associated CDK. CDK12 is a ˜164 kDa protein consisting of 1490 amino acids, which is encoded by CDK12 gene. Human CDK12 gene located in human chromosome 17q12 and composed of 14 exons. And the Gene ID of human CDK12 gene is 51755. CDK12 complexes with cyclin K to regulate gene transcription elongation via phosphorylating RNA polymerase II and also regulates translation. CDK12 has become an attractive therapeutic target for tumor treatment.

The term “THZ531”, as used herein, generally refers to a selective covalent inhibitor of CDK12 and CDK13. The chemical name of THZ531 is (R,E)-N-(4-(3-((5-chloro-4-(1H-indol-3-yl)pyrimidin-2-yl)amino)piperidine-1-carbonyl)phenyl)-4-(dimethylamino)but-2-enamide; and the CAS number thereof is 1702809-17-3. THZ531 is a compound having the following formula:

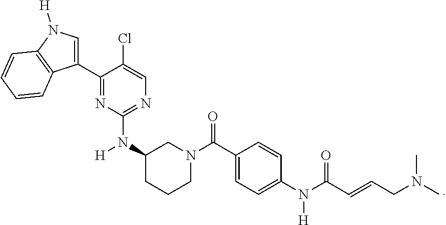 [SMILES: CN(C)C/C=C/C(=O)Nc1ccc(C(=O)N2CCC[C@@H](Nc3ncc(Cl)c(-c4c[nH]c5ccccc45)n3)C2)cc1]

The term “Dinaciclib” or “SCH-727965”, as used herein, generally refers to a multiple CDK inhibitor. Dinaciclib may inhibit CDK1, CDK2, CDK5, CDK9 and CDK12. The CAS number of Dinaciclib is 779353-014. Dinaciclib is a compound having the following formula:

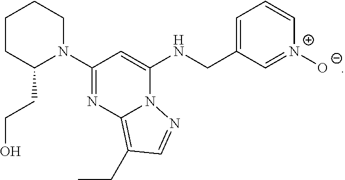 [SMILES: CCc1cnn2c(NCc3ccc[n+]([O-])c3)cc(N3CCCC[C@H]3CCO)nc12 ; "."]

The term “SR-3029”, as used herein, generally refers to a potent CK1δ/CK1ε inhibitor. It may reduce the expression of the Wnt/β-catenin target CCND1 and may decrease protein levels of nuclear β-catenin and cyclin D1. SR-3029 may inhibit Cdk4/cyclin D1, Cdk4/cyclin D3, Cdk6/cyclin D1, Cdk6/cyclin D3 and CDK12. And the CAS number thereof is 1454585-06-8. SR-3029 is a compound having the following formula:

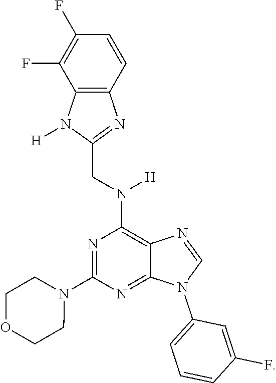 [SMILES: Fc1cccc(-n2cnc3c(NCc4nc5ccc(F)c(F)c5[nH]4)nc(N4CCOCC4)nc32)c1]

The term “immune checkpoint inhibitor”, as used herein, generally refers to any agent that can completely or partially reduce, inhibit, interfere with, or modulate one or more immune checkpoint proteins that regulate T cell activation or function. For example, the immune checkpoint inhibitor may interfere or/and prevent the interaction of PD-1 with its ligand PD-L1: the immune checkpoint inhibitor may interfere or/and prevent the interaction of CTLA4 with its ligand.

The term “ISVD”, as used herein, generally refers to antigen-binding domains or fragments such as VHH domains or VH or VL domains, respectively. The terms antigen-binding molecules or antigen-binding protein are used interchangeably and may also comprise Nanobodies. The immunoglobulin single variable domains further are light chain variable domain sequences (e.g., a VL-sequence), or heavy chain variable domain sequences (e.g., a VH-sequence); more specifically, they may be heavy chain variable domain sequences that are derived from a conventional four-chain antibody or heavy chain variable domain sequences that are derived from a heavy chain antibody. Accordingly, the immunoglobulin single variable domains may be domain antibodies, or immunoglobulin sequences that are suitable for using as domain antibodies; single domain antibodies, or immunoglobulin sequences that are suitable for using as single domain antibodies; “dAbs,” or immunoglobulin sequences that are suitable for using as dAbs; or Nanobodies, which may comprise a VHH sequence, the ISVD may comprise sequences originating from fully human, humanized, otherwise sequence optimized and/or chimeric immunoglobulins, the ISVD may comprise four framework regions or “FRs,” which are referred to in the art and herein as “Framework region 1” or “FR1”; as “Framework region 2” or “FR2”; as “Framework region 3” or “FR3”; and as “Framework region 4” or “FR4,” respectively; which framework regions are interrupted by three complementary determining regions or “CDRs,” which are referred to in the art as “Complementarity Determining Region 1” or “CDR1”; as “Complementarity Determining Region 2” or “CDR2”; and as “Complementarity Determining Region 3” or “CDR3,” respectively.

The term “antigen binding portion”, as used herein, generally refers to one or more portions of a full-length antibody, the antigen binding portion maintains the ability of binding to an antigen (such as HER2) that is the same as that bound by the antibody, and competes with the full-length antibody for the specific binding to an antigen. General reference is made to Fundamental Immunology, Ch. 7 (Paul, W., ed., edition II, Raven Press, N.Y. (1989), which is incorporated herein by reference in its entirety and for all purposes. The antigen binding portion may be produced using a recombinant DNA technology or through enzymatic or chemical breakage of a full-length antibody. For example, the antigen binding portion may comprise a polypeptide such as a Fab, a Fab′, a F(ab′)₂, a Fd, a Fv, a dAb, a complementary determining region (CDR) fragment, a single-chain antibody (such as a scFv), a chimeric antibody, and a diabody, and it may comprise at least the part of the antibody sufficiently endowing the polypeptide with the specific antigen binding ability. In present application, the antigen binding portion of the antibody may be obtained from a given antibody using a conventional technology (such as recombinant DNA technology or enzymatic or chemical breakage process) known to those skilled in the art, and are screened for its specificity in a process that is the same for screening full-length antibodies.

The term “dimer”, as used herein, generally refers to a macromolecular complex formed by two, usually non-covalently bound, monomer units. Each monomer unit may be a macromolecule, such as a polypeptide chain or a polynucleotide. The term “homodimer,” as used herein, generally refers to a dimer composed of or formed by two substantially identical monomers, such as two substantially identical polypeptide chains. For example, the two monomers of a homodimer may be different at one or more regions or positions, however, such difference does not cause significant alteration in the function or structure of the monomer. For example, a person skilled in the art would consider the difference between the two monomers to be of little or no biological and/or statistical significance within the context of the biological characteristic considered in the present application. The structural/compositional difference between the two monomers may be, for example, less than about 50%, less than about 40%, less than about 30%, less than about 20%, less than about 10%, less than about 5%, or less.

The term “CD80,” as used herein, generally refers to a ligand for CD28/CTLA4, also known as B7.1, its functional variant and/or its functional fragments. The term “CD80” may comprise a polypeptide or a fragment thereof having at least about 85% amino acid sequence identity to NCBI Accession No. P33681 and that specifically binds CTLA4. Also included within the definition of CD80 are variants which vary in amino acid sequence from naturally occurring CD80 but which retain the ability to specifically bind to CTLA4. CD80 sequences are known in the art and are provided, for example, at GenBank Accession Numbers P33681. The term “CD80” as used herein comprises human CD80 (hCD80), variants, isoforms, and species homologs of hCD80, and analogs having at least one common epitope with hCD80. For example, the term “CD80” also encompasses CD80 from other species, such as other mammals, for example, rat, mouse, rabbit, non-human primate, pig, or bovine. The complete hCD80 sequence has a GenBank login No. P33681.

The term “CD86,” as used herein, generally refers to a ligand for CD28/CTLA4, also known as B7.2, its functional variant and/or its functional fragments. The term “CD86” may comprise a polypeptide or a fragment thereof having at least about 85% amino acid sequence identity to NCBI Accession No. P42081 and that specifically binds CTLA4. Also included within the definition of CD86 are variants which vary in amino acid sequence from naturally occurring CD86 but which retain the ability to specifically bind to CTLA4. CD86 sequences are known in the art and are provided, for example, at GenBank Accession Numbers U04343. The term “CD86” as used herein may comprise human CD86 (hCD86), variants, isoforms, and species homologs of hCD86, and analogs having at least one common epitope with hCD86. For example, the term “CD86” may also comprise CD86 from other species, such as other mammals, for example, rat, mouse, rabbit, non-human primate, pig, or bovine. The complete hCD86 sequence has a GenBank login No. U04343.

The term “PD1,” as used herein, generally refers to programmed death-1 receptor, also known as CD279, its functional variant and/or its functional fragments. Also included within the definition of PD1 are variants which vary in amino acid sequence from naturally occurring PD1 but which retain the ability to specifically bind to PD-L1. PD1 sequences are known in the art and are provided, for example, at GenBank Accession Number Q15116.3. The term “PD1” as used herein may comprise human PD1 (hPD1), variants, isoforms, and species homologs of hPD1, and analogs having at least one common epitope with hPD1. For example, the term “PD1” may also comprise PD1 from other species, such as other mammals, for example, rat, mouse, rabbit, non-human primate, pig, or bovine. The complete hPD1 sequence has a GenBank login No. Q15116.3.

The term “a”, as used herein, generally not means to limit as a singular. In certain embodiments, the term “a” may refer to a plural form. As used throughout this disclosure, the singular forms “a,” “an,” and “the” include plural reference unless the context clearly dictates otherwise.

The term “about”, as used herein, generally refers to a variation that is within a range of normal tolerance in the art, and generally means within ±10%, such as within 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

HER2 Inhibitor

In the present application, the HER2 inhibitor may be capable of inhibiting human HER2. For example, said HER2 inhibitor may be a HER2 antibody or an antigen binding portion thereof and/or a conjugate thereof.

For example, the HER2 inhibitor may be selected from a group consisting of Pertuzumab. Trastuzumab and Margetuximab.

For example, the HER2 inhibitor may be selected from a group consisting of DS8201a and T-DM1.

In present application, the HER2 inhibitor may be a bispecific antibody or an antigen binding portion thereof, and may capable of binding to different epitopes of human HER2, be epitopes of human HER2 may refer to “Epitope Mapping of Human HER2 Specific Mouse Monoclonal Antibodies Using Recombinant Extracellular Subdomains”, Asian Pac J Cancer Prev. 2017; 18(11): 3103-3110.

For example, the HER2 inhibitor may be a bispecific antibody or the antigen binding portion thereof, and the bispecific antibody or the antigen binding portion thereof may have a first light chain and a second light chain, and the first light chain and the second light chain may comprise a same amino acid sequence. For example, as the first light chain and the second light chain comprises the same amino acid sequence, the bispecific antibody or the antigen binding portion thereof may have a common light chain.

For example, the common light chain may be obtained via engineering from two different original monoclonal antibodies which may capable of binding to different epitopes of human HER2, respectively. In some cases, the common light chain may be originated from the light chain of either of the two original monoclonal antibodies. In some cases, the common light chain may be modified on the basis of the light chain of either of the two original monoclonal antibodies.

For example, the modification may comprise an insertion, a deletion and/or a substitution in at least one amino acid positions of an amino acid sequence of the light chain of either of the two original monoclonal antibodies. In some cases, the purpose of the modification is to maintain the affinity between the bispecific antibody or the antigen binding portion thereof to the corresponding epitopes.

In present application, light chain constant regions of the bispecific antibody or the antigen binding portion thereof may be of κ type or λ type; the κ-type light chain constant region may comprise various allotypes, such as Km1, Km2 and Km3; the λ-type light chain constant region may comprise various allotypes, such as CL1, CL2, CL3, CL6 and CL7.

In present application, the HER2 inhibitor may be a bispecific antibody or the antigen binding portion thereof, and the bispecific antibody or the antigen binding portion thereof may have a first heavy chain and a second heavy chain.

In present application, the first heavy chain and the second heavy chain are capable of correctly assembling with the light chains respectively under physiological conditions or during in vitro protein expression.

For example, the first light chain and the second light chain may be capable of assembling with a heavy chain of Pertuzumab and a heavy chain of Trastuzumab, respectively.

For example, variable region of the first light chain and/or the second light chain may comprise an amino acid sequence as set forth in any one of SEQ ID NO: 91-96. For example, variable region of the first light chain and/or the second light chain may comprise an amino acid sequence as set forth in SEQ ID NO: 91.

For example, the first light chain and the second light chain may be selected from a light chain of Pertuzumab or a mutant thereof, a light chain of Trastuzumab or a mutant thereof, respectively.

For example, variable region of the first light chain and variable region of the second light chain may be the variable region of light chain of Trastuzumab. For example, the first light chain and the second light chain may be the light chain of Trastuzumab.

For example, the first light chain and the second light chain may comprise an amino acid sequence as set forth in any one of SEQ ID NO: 65-70. For example, the first light chain and the second light chain may comprise an amino acid sequence as set forth in SEQ ID NO: 65.

For example, variable region of the first heavy chain may be a heavy chain variable region of Pertuzumab, and variable region of the second heavy chain may be a heavy chain variable region of Trastuzumab. For example, variable region of the first heavy chain may comprise an amino acid sequence as set forth in SEQ ID NO: 87; and variable region of the second heavy chain may comprise an amino acid sequence as set forth in SEQ ID NO: 88.

In the present application, the first heavy chain and/or the second heavy chain may comprise a constant region. For example, the constant region may be originated from human IgG constant region. For example, heavy chain constant region of the first heavy chain and heavy chain constant region of the second heavy chain may be identical to or different from each other. In some cases, the amino acid sequences of the variable region and the CHI domain of the first heavy chain and the second heavy chain are identical to those of the original monoclonal antibodies.

In the present application, the bispecific antibody or the antigen binding portion thereof may block both ligand-dependent and ligand-independent HER2 signaling pathway. For example, the IgG1 Fc fragment of the bispecific antibody or the antigen binding portion thereof may bind to FcRγIIIa and may mediate potent ADCC effect. For example, the bispecific antibody or the antigen binding portion thereof may enhance a HER2 internalization and/or may show better antitumor activity in preclinical models than using the original monoclonal antibodies alone, e.g. trastuzumab and pertuzumab.

In some cases, the light chain constant region and/or the heavy chain constant region of the bispecific antibody or the antigen binding portion thereof may comprise a modification in order to obtain a better ADCC, CDC, endocytosis, stability, immunogenicity and/or half-life; furthermore, the modification may also facilitate formation of the heterodimer protein during antibody expression. In the present application, technologies for modifying an Fc fragment of the heavy chain are known in the art.

For example, Fc fragment of the first heavy chain may comprise an amino acid sequence as set forth in SEQ ID NO: 89; and Fc fragment of the second heavy chain may comprise an amino acid sequence as set forth in SEQ ID NO: 90.

For example, Fc fragment of the first heavy chain may comprise an amino acid sequence as set forth in SEQ ID NO: 101; and Fc fragment of the second heavy chain may comprise an amino acid sequence as set forth in SEQ ID NO: 102.

For example, Fc fragment of the first heavy chain may comprise an amino acid sequence as set forth in SEQ ID NO: 103; and Fc fragment of the second heavy chain may comprise an amino acid sequence as set forth in SEQ ID NO: 104.

For example, the first heavy chain may comprise an amino acid sequence as set forth in SEQ ID NO: 80; and the second heavy chain may comprise an amino acid sequence as set forth in SEQ ID NO: 81.

For example, the first heavy chain may comprise an amino acid sequence as set forth in SEQ ID NO: 83; and the second heavy chain may comprise an amino acid sequence as set forth in SEQ ID NO: 84.

For example, the first heavy chain may comprise an amino acid sequence as set forth in SEQ ID NO: 97; and the second heavy chain may comprise an amino acid sequence as set forth in SEQ ID NO: 98.

For example, the first heavy chain may comprise an amino acid sequence as set forth in SEQ ID NO: 99, and the second heavy chain may comprise an amino acid sequence as set forth in SEQ ID NO: 100.

In present application, the HER2 inhibitor may be a an anti-HER2 bispecific antibody, the anti-HER2 bispecific antibody may comprise a first light chain, a second light chain, a first heavy chain and a second heavy chain, variable region of the first light chain and/or the second light chain may comprise a sequence as set forth in SEQ ID NO: 65: the variable region of the first heavy chain may comprise an amino acid sequence as set forth in SEQ ID NO: 87; and variable region of the second heavy chain may comprise an amino acid sequence as set forth in SEQ ID NO: 88. The first heavy chain may comprise an amino acid sequence as set forth in SEQ ID NO: 97; and the second heavy chain may comprise an amino acid sequence as set forth in SEQ ID NO: 98.

The amino acid sequence in the present application may also comprise an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 1-104 in the sequence listing. For example, the amino acid sequence in the present application may comprise an amino acid sequence having one or more (e.g., 1-2, 1-3, 1-4, 1-5, 1-6, 1-7, 1-8, 1-9, 1-10, or more) amino acid deletion, insertion and/or substitution in the amino acid sequence as set forth in any one of SEQ ID NO: 1-104 in the sequence listing.

Immune Checkpoint Inhibitor

In present application, the immune checkpoint inhibitor may inhibitor at least one immune checkpoint. The immune checkpoint may comprise stimulatory checkpoint molecules and inhibitory checkpoint molecules. The immune checkpoint may comprise A2AR, B7-H3, B7-H4, BTLA, CTLA-4, IDO, KIR, LAG3, NOX2, PD-1 and/or TIM3.

The immune checkpoint may be capable of specifically binding to PD-L1 and CTLA4.

In some embodiments, the immune checkpoint inhibitor may be a bispecific antibody or an antigen binding fragment thereof. For example, the immune checkpoint inhibitor may be an antigen binding fragment, and may comprise Fab, Fab′, F(ab)2, Fv fragment, F(ab′)2, scFv, di-scFv and/or dAb. For example, the immune checkpoint inhibitor may be a bispecific antibody and may be a fully human antibody.

In some embodiments, the immune checkpoint inhibitor may be a dimer. The dimer may be formed by two polypeptide chains, with each of the two polypeptide chains comprising an antibody Fc subunit.

For example, the dimer may consist of two polypeptide chains with each polypeptide chain comprising an antibody Fc subunit, and the antibody Fc subunit of one polypeptide chain may associate with the antibody Fc subunit of the other polypeptide chain to form the dimer. For example, the two polypeptide chains of the dimer may not be fused (e.g., via a peptide linker or by a peptide bond) with each other to become one single polypeptide chain.

The dimer may comprise two or more immunoglobulin single variable domains (ISVDs). For example, one polypeptide chain of the dimer may comprise two or more ISVDs, and the other polypeptide chain of the dimer may not comprise any ISVD. In another example, each of the two polypeptide chains may comprise one or more ISVDs. In another example, each of the two polypeptide chains may comprise two or more ISVDs.

At least one of the ISVDs may be specific for PD-L1, and at least one of the ISVDs may be specific for CTLA4. For example, one polypeptide chain of the dimer may comprise one or more ISVDs specific for PD-L1 and one or more ISVDs specific for CTLA4, and the other polypeptide chain of the dimer may not comprise any ISVD. In another example, one polypeptide chain of the dimer may comprise one or more ISVDs specific for PD-L1, and the other polypeptide chain of the dimer may comprise one or more ISVDs specific for CTLA4. In another example, one polypeptide chain of the dimer may comprise one or more ISVDs specific for PD-L1 and one or more ISVDs specific for CTLA4, and the other polypeptide chain of the dimer may comprise one or more ISVDs specific for PD-L1 and/or one or more ISVDs specific for CTLA4.

The one or more ISVDs specific for PD-L1 may be identical or different. The one or more ISVDs specific for CTLA4 may be identical or different.

For example, the ISVD specific for PD-L1 may not comprise any antibody light chain CDR. For example, the ISVD specific for PD-L1 may not comprise any antibody light chain variable region. For example, the ISVD specific for PD-L1 may not comprise any antibody light chain or any fragment thereof. For example, the ISVD specific for PD-L1 may comprise at least heavy chain CDR3. For example, the ISVD specific for PD-L1 may comprise heavy chain CDR1. For example, the ISVD specific for PD-L1 may comprise heavy chain CDR2. For example, the ISVD specific for PD-L1 may comprise a heavy chain variable region. For example, the ISVD specific for PD-L1 may be an anti-PD-L1 VHH. The ISVD specific for PD-L1 may be humanized.

For example, the ISVD specific for CTLA4 may not comprise any antibody light chain CDR. For example, the ISVD specific for CTLA4 may not comprise any antibody light chain variable region. For example, the ISVD specific for CTLA4 may not comprise any antibody light chain or any fragment thereof. For example, the ISVD specific for CTLA4 may comprise at least heavy chain CDR3. For example, the ISVD specific for CTLA4 may comprise heavy chain CDR1. For example, the ISVD specific for CTLA4 may comprise heavy chain CDR2. For example, the ISVD specific for CTLA4 may comprise a heavy chain variable region. For example, the ISVD specific for CTLA4 may be an anti-CTLA4 VHH. The ISVD specific for CTLA4 may be humanized.

For example, at least one of the two polypeptide chains may comprise both an ISVD specific for PD-L1 and an ISVD specific for CTLA4. For example, one of the two polypeptide chains may comprise one or more ISVDs specific for PD-L1 and one or more ISVDs specific for CTLA4. In another example, each of the two polypeptide chains may comprise one or more ISVDs specific for PD-L1 and one or more ISVDs specific for CTLA4.

For one or both of the two polypeptide chains, the ISVD specific for PD-L1 may be fused to the ISVD specific for CTLA4, optionally via a linker. For example, in one or both of the two polypeptide chains, there may be one or more ISVDs specific for PD-L1, and one or more ISVDs specific for CTLA4. When two or more ISVDs specific for PD-L1 are present in a single polypeptide chain, they may be fused to each other (e.g., directly or via a peptide linker), and one or more of them may further be fused to one or more ISVDs specific for CTLA4. When two or more ISVDs specific for CTLA4 are present in a single polypeptide chain, they may be fused to each other (e.g., directly or via a peptide linker), and one or more of them may further be fused to one or more ISVDs specific for PD-L1. One or more linkers (e.g., peptide linker) may be presented between any two ISVDs, for example, between two ISVDs specific for PD-L1, between two ISVDs specific for CTLA4, or between one ISVD specific from PD-L1 and one ISVD specific for CTLA4.

For one or both of the two polypeptide chains, the ISVD specific for PD-L1 may be fused to the ISVD specific for CTLA4, optionally via a linker; and the ISVD specific for CTLA4 may in turn be fused to the antibody Fc subunit, optionally via a linker. For example, in a single polypeptide chain, the ISVD specific for PD-L1 may be fused to the ISVD specific for CTLA4 directly (e.g., in frame) or via a linker, and the ISVD specific for CTLA4 may be fused to the antibody Fc subunit directly (e.g., in frame) or via a linker. When there are more than one ISVDs specific for PD-L1 and/or more than one ISVDs specific for CTLA4 in a single polypeptide chain, the ISVDs specific for PD-L1 and the ISVDs specific for CTLA4 may be fused directly or via a linker to each other according to any order, and at least one ISVD specific for CTLA4 may be fused to the antibody Fc subunit directly (e.g., in frame) or via a linker. For example, for one or both of the two polypeptide chains, C terminus of the ISVD specific for PD-L1 may be fused to N terminus of the ISVD specific for CTLA4, optionally via a linker; and C terminus of the ISVD specific for CTLA4 may be fused to N terminus of the antibody Fc subunit, optionally via a linker. For example, in a single polypeptide chain, C terminus of one of the ISVDs specific for PD-L1 may be fused to N terminus of one of the ISVDs specific for CTLA4, either directly (e.g., in frame) or via a linker, and C terminus of one of the ISVDs specific for CTLA4 may be fused to N terminus of the antibody Fc subunit, either directly (e.g., in frame) or via a linker. For example, when there are more than one ISVDs specific for PD-L1 and/or more than one ISVDs specific for CTLA4 in a single polypeptide chain, the ISVDs specific for PD-L1 and the ISVDs specific for CTLA4 may be fused directly or via a linker to each other according to any order, however, C terminus of at least one ISVD specific for PD-L1 may be fused to N terminus of at least one ISVD specific for CTLA4, either directly (e.g., in frame) or via a linker, and C terminus of at least one ISVD specific for CTLA4 may be fused to N terminus of the antibody Fc subunit, either directly (e.g., in frame) or via a linker.

For one or both of the two polypeptide chains, the ISVD specific for CTLA4 may be fused to the ISVD specific for PD-L1, optionally via a linker; and the ISVD specific for PD-L1 may in turn be fused to the antibody Fc subunit, optionally via a linker. For example, in a single polypeptide chain, the ISVD specific for CTLA4 may be fused to the ISVD specific for PD-L1 directly (e.g., in frame) or via a linker, and the ISVD specific for PD-L1 may be fused to the antibody Fc subunit directly (e.g., in frame) or via a linker. When there are more than one ISVDs specific for PD-L1 and/or more than one ISVDs specific for CTLA4 in a single polypeptide chain, the ISVDs specific for PD-L1 and the ISVDs specific for CTLA4 may be fused directly or via a linker to each other according to any order, and at least one ISVD specific for PD-L1 may be fused to the antibody Fc subunit directly (e.g., in frame) or via a linker. For example, for one or both of the two polypeptide chains, C terminus of the ISVD specific for CTLA4 may be fused to N terminus of the ISVD specific for PD-L1, optionally via a linker; and C terminus of the ISVD specific for PD-L1 may be fused to N terminus of the antibody Fc subunit, optionally via a linker. For example, in a single polypeptide chain, C terminus of one of the ISVDs specific for CTLA4 may be fused to N terminus of one of the ISVDs specific for PD-L1, either directly (e.g., in frame) or via a linker, and C terminus of one of the ISVDs specific for PD-L1 may be fused to N terminus of the antibody Fc subunit, either directly (e.g., in frame) or via a linker. For example, when there are more than one ISVDs specific for PD-L1 and/or more than one ISVDs specific for CTLA4 in a single polypeptide chain, the ISVDs specific for PD-L1 and the ISVDs specific for CTLA4 may be fused directly or via a linker to each other according to any order, however, C terminus of at least one ISVD specific for CTLA4 may be fused to N terminus of at least one ISVD specific for PD-L1, either directly (e.g., in frame) or via a linker, and C terminus of at least one ISVD specific for PD-L1 may be fused to N terminus of the antibody Fc subunit, either directly (e.g., in frame) or via a linker.

The linker (e.g., a peptide linker) employed in the present application (e.g., as comprised by the dimer of the present application) may be a synthetic amino acid sequence that connects or links two polypeptide sequences, e.g., via peptide bonds. For example, the peptide linker may comprise 1-10 amino acids (e.g., 1, 2, 3, 4, 5, 6, 7, 8, 9, 10 or more amino acids), 1-15 amino acids (e.g., 1-10, 11, 12, 13, 14 or 15 amino acids), 1-20 amino acids (e.g., 1-15, 16, 17, 18, 19, or 20 amino acids). 1-30 amino acids or more (e.g., 1-20, 21, 22, 23, 24, 25, 26, 27, 28, 29, 30 or more amino acids). For example, the peptide linker may comprise an amino acid sequence as set forth in any of SEQ ID NO: 33-34.

The antibody Fc subunit may be derived from an IgG Fc subunit. For example, the IgG may be selected from the group consisting of IgG1, IgG2, IgG3 and IgG4. In some embodiments, the IgG is a human IgG1, and the IgG Fc subunit is a human IgG1 Fc subunit. In some embodiments, the Fc subunit comprises an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 35, 38 and 39. For example, the Fc subunit may comprise an amino acid sequence having one or more (e.g., 1-2, 1-3, 1-4, 1-5, 1-6, 1-7, 1-8, 1-9, 1-10, or more) amino acid deletion, insertion and/or substitution in the amino acid sequence as set forth in any one of SEQ ID NO: 35, 38 and 39.

In some embodiments, the Fc subunit may be a variant of the IgG Fc subunit (e.g., a variant of the human IgG1 Fc subunit). For example, the variant may comprise one or more amino acid mutations that enhance or reduce the ADCC or CDC activities. As another example, the variant may comprise one or more amino acid mutations that affect FcRn binding activity and/or the half-life of the molecule comprising the variant. As yet another example, the variant may comprise one or more amino acid mutations that affect an interaction (e.g., association) between two or more Fc subunits (or Fc monomers) and/or increase or decrease an efficiency of Fc heterodimer formation, for example, the variant may comprise one or more of the amino acid substitutions as described in CN102558355A, CN103388013A, CN105820251A, or CN106883297A, each of which is incorporated by reference herein.

The ISVD specific for PD-L1 may be capable of specifically binding to human PD-L1. For example, the ISVD specific for PD-L1 may be capable of specifically binding to an epitope in an extracellular domain of the human PD-L1. Such epitopes are known in the art, for example, as shown by Gang Hao et al., J. Mol. Recognit. 2015; 28: 269-276, Zhang et al., Oncotarget. 2017 October; 08 (52): 90215-90224, and Zhang et al., Cell Discov. 2017 Mar. 7: 3:17004.

For example, the ISVD specific for PD-L1 may be capable of binding to N-terminal IgV domain of human PD-L1. The N-terminal IgV domain of human PD-L1 (including the signal peptide) may comprise an amino acid sequence as set forth in SEQ ID NO: 64. In the present application, the ISVD specific for PD-L1 may be capable of binding to residues I54, Y56, E58, Q66 and/or R113 of human PD-L1 N-terminal IgV domain. In a specific embodiment, the ISVD specific for PD-L1 is capable of binding to residues I54, Y56, E58, Q66 and R113 of human PD-L1 N-terminal IgV domain (e.g., amino acid residue I54, Y56, E58, Q66 and/or R113 of SEQ ID NO: 64). The ISVD specific for PD-L1 may be capable of further binding to residues D61, N63, V68, M115, S117, Y123 and/or R125 of human PD-L1 N-terminal IgV domain (e.g., amino acid residue D61, N63, V68, M115, S117, Y123 and/or R125 of SEQ ID NO: 64). For example, the ISVD specific for PD-L1 may be capable of binding to residues I54, Y56, E58, Q66, R113, D61, N63, V68, M115, S117, Y123 and/or R125 of human PD-L1 N-terminal IgV domain (e.g., amino acid residue I54, Y56, E58, Q66, R113, D61, N63, V68, M115, S117, Y123 and/or R125 of SEQ ID NO: 64). For example, the ISVD specific for PD-L1 is capable of binding to a conformational epitope of human PD-L1 N-terminal IgV domain, the conformational epitope may comprise residues I54, Y56, E58, Q66 and/or R113 of the human PD-L1 N-terminal IgV domain (e.g., amino acid residue I54, Y56, E58, Q66 and/or R113 of SEQ ID NO: 64). For example, the ISVD specific for PD-L1 is capable of binding to a conformational epitope of human PD-L1 N-terminal IgV domain, the conformational epitope may comprise residue I54, Y56, E58, Q66, R113, D61, N63, V68, M115, S117, Y123 and/or R125 of the human PD-L1 N-terminal IgV domain (e.g., amino acid residue I54, Y56, E58, Q66, R113, D61, N63, V68, M115, S117, Y123 and/or R125 of SEQ ID NO: 64).

The ISVDs specific for PD-L1 of the present application (e.g., PD-L1 ISVD-9 and the humanized variants thereof) may bind to the N-terminal IgV domain of human PD-L1. Taking PD-L1 ISVD-9 as an example, the residue Phe101 of PD-L1 ISVD-9 (SEQ ID NO: 6) interacts with Tyr56 of human PD-L1 N-terminal IgV domain, and when the Tyr56 of human PD-L1 N-terminal IgV domain was substituted by Ala, the binding affinity between PD-L1 ISVD-9 and PD-L1 was reduced by over 200 folds. When the Ile54 of human PD-L1 N-terminal IgV domain was substituted by Ala, the binding affinity between PD-L1 ISVD-9 and PD-L1 was reduced by about 40 folds. The residue Asp99 of PD-L1 ISVD-9 (SEQ ID NO: 6) interacts with Arg113 of human PD-L1 N-terminal IgV domain, and when the Arg113 of human PD-L1 N-terminal IgV domain was substituted by Ala, the binding affinity between PD-L1 ISVD-9 and PD-L1 was reduced by about 90 folds. The residue Ser100 of PD-L1 ISVD-9 (SEQ ID NO: 6) interacts with Glu58 of human PD-L1 N-terminal IgV domain, and when the Glu58 of human PD-L1 N-terminal IgV domain was substituted by Ala, the binding affinity between PD-L1 ISVD-9 and PD-L1 was reduced by about 25 folds. The residue Thr105 of PD-L1 ISVD-9 (SEQ ID NO: 6) interacts with Gln66 of human PD-L1 N-terminal IgV domain, and when the Gln66 of human PD-L1 N-terminal IgV domain was substituted by Ala, the binding affinity between PD-L1 ISVD-9 and PD-L1 was reduced by about 82 folds. In addition, residues D61, N63, V68, M115, S117, Y123 and R125 of human PD-L1 N-terminal IgV domain may be involved in the interaction between PD-L1 ISVD-9 and human PD-L1, substituting these residues with Ala resulted in a reduction of binding affinity by about 2-10 folds. These results are summarized in Table 1 below.

TABLE 1
Effects of Substitutions in human
PD-L1 for binding of PD-L1 ISVD-9

| Human PD-L1 | | |
|---|---|---|
| mutation | K_{D} (M) | K_{D, mutant}/K_{D, WT} |
| WT | 5.92E−09 | 1 |
| I54A | 2.42E−07 | 40.9 |
| Y56A | 1.24E−06 | 209.5 |
| E58A | 1.49E−07 | 25.2 |
| D61A | 1.99E−08 | 3.4 |
| N63A | 2.30E−08 | 3.9 |
| Q66A | 4.88E−07 | 82.4 |
| V68A | 2.76E−08 | 4.7 |
| R113A | 5.34E−07 | 90.2 |
| M115A | 5.51E−08 | 9.3 |
| S117A | 1.26E−08 | 2.1 |
| Y123A | 4.24E−08 | 7.2 |
| R125A | 2.97E−08 | 5.0 |

The ISVD specific for PD-L1 may be capable of blocking binding of PD-L1 to PD1. For example, the ISVD specific for PD-L1 may be capable of blocking binding of PD-L1 to CD80.

The ISVD specific for PD-L1 may cross-compete for binding to PD-L1 with a reference anti-PD-L1 antibody.

The reference anti-PD-L1 antibody may comprise a heavy chain CDR3. The heavy chain CDR3 may comprise an amino acid sequence as set forth in DSFX₁X₂PTCX₃X₄X₅X₆SSGAFQY (SEQ ID NO: 1), wherein X₁ may be E or G; X₂ may be D or Y; X₃ may be T or P; X₄ may be L or G; X₅ may be V or P; and X6 may be T or A. For example, the reference anti-PD-L1 antibody may comprise a heavy chain CDR3 comprising an amino acid sequence as set forth in any one of SEQ ID NO: 5 and 9. The reference anti-PD-L1 antibody may also comprise a heavy chain CDR1. The heavy chain CDR1 may comprise an amino acid sequence as set forth in GX₁X₂X₃X₄X₅RCMA (SEQ ID NO: 2), wherein X₁ may be K or N; X₂ may be M or I; X₃ may be S or I; X₄ may be S or R; and X₅ may be R or V. For example, the reference anti-PD-L1 antibody may comprise a heavy chain CDR1 comprising an amino acid sequence as set forth in any one of SEQ ID NO: 3 and 7. For example, the reference anti-PD-L1 antibody may comprise a heavy chain CDR2. The heavy chain CDR2 may comprise an amino acid sequence as set forth in any one of SEQ ID NO: 4, 8 and 11. For example, the reference anti-PD-L1 antibody is an ISVD specific for PD-L1, such as an anti-PD-L1 VHH. The reference anti-PD-L1 antibody may comprise a heavy chain variable domain. The reference anti-PD-L1 antibody may comprise a heavy chain variable domain comprising an amino acid sequence as set forth in any one of SEQ ID NO: 6, 10, 12, 13, 14 and 15. For example, the heavy chain variable domain may comprise an amino acid sequence as set forth in SEQ ID NO: 6.

In the present application, the ISVD specific for PD-L1 (e.g., as comprised in the dimer of the present application) may comprise a heavy chain CDR3. The heavy chain CDR3 may comprise an amino acid sequence as set forth in DSFX₁X₂PTCX₃X₄X₅X₆SSGAFQY (SEQ ID NO: 1), wherein X₁ may be E or G; X₂ may be D or Y; X₃ may be T or P; X₄ may be L or G; X₅ may be V or P; and X₆ may be T or A. For example, the ISVD specific for PD-L1 may comprise a heavy chain CDR3 comprising an amino acid sequence as set forth in any one of SEQ ID NO: 5 and 9.

For example, the ISVD specific for PD-L1 may comprise a heavy chain CDR3 comprising an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 5 and 9. For example, the heavy chain CDR3 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NOs: 5 and 9.

In the present application, the ISVD specific for PD-L1 (e.g., as comprised in the dimer of the present application) may also comprise a heavy chain CDR1. The heavy chain CDR1 may comprise an amino acid sequence as set forth in GX₁X₂X₃X₄X₅RCMA (SEQ ID NO: 2), wherein X₁ may be K or N; X₂ may be M or I; X₃ may be S or I; X₄ may be S or R; and X₅ may be R or V. For example, the ISVD specific for PD-L1 may comprise a heavy chain CDR1 comprising an amino acid sequence as set forth in any one of SEQ ID NO: 3 and 7.

For example, the ISVD specific for PD-L1 may comprise a heavy chain CDR1 comprising an amino acid sequence having at least 80/o (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 3 and 7. For example, the heavy chain CDR1 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NOs: 3 and 7.

In the present application, the ISVD specific for PD-L1 (e.g., as comprised in the dimer of the present application) may further comprise a heavy chain CDR2. The heavy chain CDR2 may comprise any suitable amino acid sequence. In some cases, the ISVD specific for PD-L1 may comprise a heavy chain CDR2 comprising an amino acid sequence as set forth in any one of SEQ ID NO: 4, 8 and 11.

For example, the ISVD specific for PD-L1 may comprise a heavy chain CDR2 comprising an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 4, 8 and 11. For example, the heavy chain CDR2 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NOs: 4, 8 and I1.

In the present application, the ISVD specific for PD-L1 (as comprised in the dimer of the present application) may comprise a heavy chain variable domain. The ISVD specific for PD-L1 may comprise a heavy chain variable domain comprising an amino acid sequence as set forth in any one of SEQ ID NO: 6, 10, 12, 13, 14 and 15. For example, the heavy chain variable domain may comprise an amino acid sequence as set forth in SEQ ID NO: 6.

For example, the ISVD specific for PD-L1 may comprise a heavy chain variable domain comprising an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 6, 10, 12, 13, 14 and 15. For example, the ISVD specific for PD-L1 may comprise a heavy chain variable domain comprising an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NO: 6, 10, 12, 13, 14 and 15.

In the present application, the ISVD specific for PD-L1 may comprise an amino acid sequence as set forth in any one of SEQ ID NO: 6, 10, 12, 13, 14 and 15. For example, the ISVD specific for PD-L1 (as comprised in the dimer of the present application) may comprise an amino acid sequence as set forth in SEQ ID NO: 6. For example, the ISVD specific for PD-L1 may comprise an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 6, 10, 12, 13, 14 and 15. For example, the ISVD specific for PD-L1 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NO: 6, 10, 12, 13, 14 and 15.

For example, the ISVD specific for PD-L1 may comprise or may consist of a heavy chain variable domain (VH or VHH).

For example, the ISVD specific for PD-L1 may be selected from PD-L1 ISVD-9, PD-L1 ISVD-6, PD-L1 ISVD-m3, PD-L1 ISVD-4. PD-L1 ISVD-11 and PD-L1 ISVD-13.

The ISVD specific for CTLA4 may be capable of specifically binding to human CTLA4. For example, the ISVD specific for CTLA4 may be capable of specifically binding to an epitope in an extracellular domain of the human CTLA4, such an epitope may include those described in CN107400166A, and those described by Udupi A. Ramagopal, et. al., PNAS 2017 May, 114 (21)

The ISVD specific for CTLA4 may be capable of blocking binding of CTLA4 to CD80. For example, the ISVD specific for CTLA4 may be capable of blocking binding of CTLA4 to CD86. For example, the ISVD specific for CTLA4 may be humanized.

The ISVD specific for CTLA4 may cross-compete for binding to CTLA4 with a reference anti-CTLA4 antibody.

The reference anti-CTLA4 antibody may comprise a heavy chain CDR3. The heavy chain CDR3 may comprise an amino acid sequence as set forth in SEQ ID NO: 19. The reference anti-CTLA4 antibody may also comprise a heavy chain CDR1. The heavy chain CDR1 may comprise an amino acid sequence as set forth in SEQ ID NO: 17. For example, the reference anti-CTLA4 antibody may comprise a heavy chain CDR2. The heavy chain CDR2 may comprise an amino acid sequence as set forth in AIX₁X₂GGGSTYYADSVKG (SEQ ID NO: 16), wherein X₁ may be Y or S; and X₂ may be I or L. For example, the heavy chain CDR2 may comprise an amino acid sequence as set forth in any one of SEQ ID NO: 18, 21 and 23. For example, the reference anti-CTLA4 antibody is an ISVD specific for CTLA4, such as an anti-CTLA4 VHH. The reference anti-CTLA4 antibody may comprise a heavy chain variable domain. The reference anti-CTLA4 antibody may comprise a heavy chain variable domain comprising an amino acid sequence as set forth in any one of SEQ ID NO: 20, 22, and 24-32. For example, the heavy chain variable domain may comprise an amino acid sequence as set forth in SEQ ID NO: 20.

In the present application, the ISVD specific for CTLA4 (e.g., as comprised in the dimer of the present application) may comprise a heavy chain CDR3. The heavy chain CDR3 may comprise an amino acid sequence as set forth in SEQ ID NO: 19.

For example, the ISVD specific for CTLA4 may comprise a heavy chain CDR3 comprising an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in SEQ ID NO: 19. For example, the heavy chain CDR3 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in SEQ ID NO: 19.

In the present application, the ISVD specific for CTLA4 (e.g., as comprised in the dimer of the present application) may also comprise a heavy chain CDR1. The heavy chain CDR1 may comprise an amino acid sequence as set forth in SEQ ID NO: 17.

For example, the ISVD specific for CTLA4 may comprise a heavy chain CDR1 comprising an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in SEQ ID NO: 17. For example, the heavy chain CDR1 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in SEQ ID NOs: 17.

In the present application, the ISVD specific for CTLA4 (e.g., as comprised in the dimer of the present application) may further comprise a heavy chain CDR2. The heavy chain CDR2 may comprise an amino acid sequence as set forth in AIX₁X₂GGGSTYYADSVKG (SEQ ID NO: 16), wherein X₁ may be Y or S; and X₂ may be I or L. In some case, the ISVD specific for CTLA4 may comprise a heavy chain CDR2 comprising an amino acid sequence as set forth in any one of SEQ ID NO: 18, 21 and 23.

For example, the ISVD specific for CTLA4 may comprise a heavy chain CDR2 comprising an amino acid sequence having at least 80/o (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 18, 21 and 23. For example, the heavy chain CDR2 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NOs: 18, 21 and 23.

In the present application, the ISVD specific for CTLA4 (as comprised in the dimer of the present application) may comprise a heavy chain variable domain. The ISVD specific for CTLA4 may comprise a heavy chain variable domain comprising an amino acid sequence as set forth in any one of SEQ ID NO; 20, 22, and 24-32. For example, the heavy chain variable domain may comprise an amino acid sequence as set forth in SEQ ID NO: 20.

For example, the ISVD specific for CTLA4 may comprise a heavy chain variable domain comprising an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 20, 22, and 24-32. For example, the ISVD specific for CTLA4 may comprise a heavy chain variable domain comprising an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NO: 20, 22, and 24-32.

In the present application, the ISVD specific for CTLA4 may comprise an amino acid sequence as set forth in any one of SEQ ID NO: 20, 22, and 24-32. For example, the ISVD specific for CTLA4 (as comprised in the dimer of the present application) may comprise an amino acid sequence as set forth in SEQ ID NO: 20.

For example, the ISVD specific for CTLA4 may comprise an amino acid sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 20, 22, and 24-32. For example, the ISVD specific for CTLA4 may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NO: 20, 22, and 24-32.

For example, the ISVD specific for CTLA4 comprises or consists of a heavy chain variable domain (VH or VHH).

For example, the dimer of the present application may comprise or consist of two polypeptide chains. The amino acid sequence of the two polypeptide chains may be identical or different. For example, the dimer of the present application may be homodimer.

In the present application, one or both of the two polypeptide chains of the dimer may comprise an amino acid sequence as set forth in any one of claims 40-43, 46, 48 and 50. For example, one or both of the two polypeptide chains of the dimer may comprise an amino acid sequence as set forth in SEQ ID NO: 40.

In specific examples, one or both of the two polypeptide chains of the dimer may comprise an amino acid sequence having at least 80% (e.g., at least 810%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%) identity to an amino acid sequence as set forth in any one of SEQ ID NO: 40-43, 46, 48 and 50. For example, one or both of the two polypeptide chains of the dimer may comprise an amino acid sequence having one or more (e.g., 1-2, 1-3, 1-4, 1-5, 1-6, 1-7, 1-8, 1-9, 1-10, or more) amino acid deletion, insertion and/or substitution in the sequence as set forth in any one of SEQ ID NO: 40-43, 46, 48 and 50.

For example, an ISVD specific for PD-L1 may be fused (directly or indirectly, e.g., via a linker, such as a peptide linker) to an N-terminal amino acid of an ISVD specific for CTLA4 to form a bi-specific binding moiety. Then, one such bi-specific binding moiety may be fused (directly or indirectly, e.g., via a linker, such as a peptide linker) to an N-terminal amino acid of one Fc subunit of the present application to provide one polypeptide chain of the dimer. Then, another such bi-specific binding moiety may be fused (directly or indirectly, e.g., via a linker, such as a peptide linker) to an N-terminal amino acid of another Fc subunit of the present application to provide the other polypeptide chain of the dimer. The two Fc subunits of the two polypeptide chains may associate with each other (e.g., via non-covalent interactions and/or disulfide bond or other covalent bond, For example, such covalent bond is not a peptide bond) to form the dimer. The two bi-specific binding moieties may be identical or different. The two Fc subunits may be identical or different.

For example, the dimer may be a proteinaceous homodimer comprising two identical polypeptide chains, with each polypeptide chain comprising one of the bi-specific binding moiety fused to one of the Fc subunits, and the two Fc subunits associate with each other to form the proteinaceous homodimer. The two Fc subunits may associate with each other via non-covalent interactions and/or disulfide bond or other covalent bond, For example, such covalent bond is not a peptide bond.

The dimer of the present application may be capable of competing with CD80 and/or CD86 for binding to CTLA4. For example, the competition may be examined in an in vitro experiment using CTLA4 expressing cell lines, such as a CTLA4 expressing HEK293 cell line. As another example, the competition may be examined in an ELISA essay, such as a competition ELISA assay.

The dimer of the present application may be capable of competing with PD1 and/or CD80 for binding to PD-L1. For example, the competition may be examined in an in vitro experiment using PD-L1 expressing cell lines, such as a PD-L1 expressing A375 cell line. As another example, the competition may be examined in an ELISA essay, such as a competition ELISA assay.

The dimer of the present application may be capable of blocking binding of PD-L1 to PD-1. For example, the dimer of the present application may be capable of blocking binding of PD-L1 to CD80. For example, the dimer of the present application may be capable of blocking binding of CTLA4 to CD80. For example, the dimer of the present application may be capable of blocking binding of CTLA4 to CD86.

The dimer of the present application may bind to CTLA4 with a KD of a value no more than about 1×10⁻⁶ M, for example, no more than about 1×10⁻⁷ M, no more than about 1×10⁻⁸ M, no more than about 0.5×10⁻⁸ M, no more than about 1×10⁻⁹ M, no more than about 1×10⁻¹⁰ M or lower.

The dimer of the present application may bind to PD-L1 with a KD of a value no more than about 1×10⁻⁶ M, for example, no more than about 1×10⁻⁷ M, no more than about 1×10⁻⁸ M, no more than about 0.5×10⁻⁸ M, no more than about 1×10⁻⁹ M, no more than about 1×10⁻¹⁰ M or lower.

The dimer of the present application may be capable of stimulating the secretion of an immunoregulator (e.g., IL-2) by immune cells (e.g., PBMC cells).

For example, the immune checkpoint inhibitor of the present application may comprise the ISVD specific for CTLA4 and the ISVD specific for PDL1. The ISVD specific for PD-L1 may comprise the CDR3 comprising an amino acid sequence as set forth in SEQ ID NO. 5, the CDR2 comprising an amino acid sequence as set forth in SEQ ID NO. 4, the CDR1 comprising an amino acid sequence as set forth in SEQ ID NO. 3. And the ISVD specific for CTLA4 may comprise the CDR3 comprising an amino acid sequence as set forth in SEQ ID NO. 19, the CDR2 comprising an amino acid sequence as set forth in SEQ ID NO. 18, and the CDR1 comprising an amino acid sequence as set forth in SEQ ID NO. 17. And the dimer of the present application may comprise the ISVD specific for PD-L1 comprising an amino acid sequence as set forth in SEQ ID NO.6, and the ISVD specific for CLTA4 comprising an amino acid sequence as set forth in SEQ ID NO. 20. For example, the dimer of the present application may comprise an amino acid sequence of SEQ ID NO.40.

Multiple CDK Inhibitor

In the present application, the multiple CDK inhibitor may be capable of inhibiting at least one kind of CDK. For example, the multiple CDK inhibitor may be capable of inhibiting CDK1, CDK2, CDK5, CDK9 and/or CDK12. For example, the multiple CDK inhibitor may be capable of inhibiting CDK12. For example, the multiple CDK inhibitor may be not capable of inhibiting CDK4. For example, the multiple CDK inhibitor may be not capable of inhibiting CDK6.

In present application, the CDK12 may be human CDK12. For example, said the multiple CDK inhibitor may comprise a CDK12 inhibitor. For example, said multiple CDK inhibitor may directly or indirectly reduce and/or inhibit the activity of the corresponding one or more CDKs. For example, said multiple CDK inhibitor may directly or indirectly reduce and/or inhibit the activity of CDK12.

In present application, the multiple CDK inhibitor may be selected from a group consisting of: THZ531, Dinaciclib and SR-3029.

Treating Method and the Medicinal Product

In one aspect, the present application provides a method of preventing, alleviating or treating tumor or inhibiting tumor growth in a subject, comprising: administrating to the subject the HER2 inhibitor of present application, wherein the subject comprises an alteration in a protein and/or a gene encoding the protein, and the protein comprises HER2 and/or CDK12.

In another aspect, the present application provides a method of preventing, alleviating or treating a tumor or inhibiting the solid tumor growth in a subject in need of, comprising: administrating to the subject the HER2 inhibitor of present application, wherein the tumor is CDK12-amplified tumor.

In the present application, the alteration may comprise a mutation, an amplification, a fusion and/or a rearrangement in the HER2 gene and/or CDK12 gene.

For example, the alteration may comprise a mutation in the protein, and/or a mutation in the gene. For example, the mutation may be illustrated as any change in the amino acid sequence of the protein. For example, the mutation may be illustrated any change in the nucleotide sequence of the gene.

For example, the alteration may comprise mutations of at least one amino acid in the amino acid sequence of the protein. For example, the alteration may comprise at least one mutation of said HER2 protein. For example, the mutation may comprise T862A, H878Y and/or R897W. For example, the mutation may comprise T862A, H878Y and R897W.

In present application, T862A represents the original amino acid T at the 862th position is replaced with the amino acid A. In present application, the position of the amino acid is numbered from the N terminal of human HER2 protein. The amino acid sequence of the human HER2 protein may be referred to UniProtKB/Swiss-Prot: P04626.1. For example, the first amino acid from the N terminal of human HER2 protein is M, and the 862th amino acid from the N terminal of human HER2 protein is T.

In the present application, the alteration may comprise an amplification in the gene, for example, the HER2 gene and/or CDK12 gene. For example, the amplification may comprise an enhanced DNA copies of the gene.

In the present application, the alteration may comprise an amplification in the protein and/or the mRNA encoding the protein. For example, the HER2 and/or CDK12. For example, the amplification may comprise an enhanced mRNA copies and/or an enhanced mRNA expression level. For another example, the amplification may comprise an enhanced protein expression level of the protein.

For example, the alteration may comprise an alteration of CDK12 gene. For example, the alteration may comprise an amplification of CDK12 gene. For example, the alteration may comprise an enhanced DNA copy of CDK12 gene. For example, the alteration may comprise an enhanced mRNA copies encoded by CDK12 gene. For example, the alteration may comprise an enhanced protein expression level of CDK12 protein.

For example, the alteration may comprise an alteration of HER2 gene. For example, the alteration may comprise an amplification of HER2 gene. For example, the alteration may comprise an enhanced DNA copy of HER2 gene. For example, the alteration may comprise an enhanced mRNA copies encoded by HER2 gene. For example, the alteration may comprise an enhanced protein expression level of HER2 protein.

For example, the alteration may comprise a co-amplification of CDK12 gene and HER2 gene. For example, the amplification may comprise an enhanced DNA copy of CDK12 gene and HER2 gene. For example, the amplification may comprise an enhanced mRNA copies encoded by CDK12 gene and HER2 gene. For example, the amplification may comprise an enhanced protein expression level of CDK12 protein and HER2 protein.

In the present application, the enhanced may represent enhancing by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 500% or more compared to that of a normal cell. For example, the normal cell may be from a healthy subject, and/or from a tissue without being affected by any tumor.

For example, the subject may be not responsive to a conventional therapy for HER2-related tumor. For example, the conventional therapy for HER2-related tumor may comprise administrating drugs which is specific for targeting HER2. For example, may comprise HER2 antigen binding protein (for example, anti-HER2 antibody), the conjugate thereof and/or HER2 specific inhibitors. For example, the conventional therapy for HER2-related tumor may comprise administrating HER2-ADC, pyrotinib, neratinib, tucatinib, trastuzumab and/or pertuzumab. For example, the conventional therapy for HER2-related tumor may comprise administrating drugs which is universal for treating tumor. For example, may comprise any available chemotherapy drugs. For example, the conventional therapy for HER2-related tumor may comprise administrating docetaxel, capecitabine and/or lapatinib.

In present application, the not responsive may refer to a syndrome of the tumor of the subject has not been alleviated significantly after administrated with the conventional therapy for HER2-related tumor. For example, the syndrome may comprise a decrease of the volume of a tumor. For example, the syndrome may comprise an extension of the OS, ORR and/or PFS.

In the present application, the tumor may comprise a solid tumor. For example, the tumor may comprise metastatic tumor, early tumor and/or locally advanced tumor. For example, the tumor may comprise HER2 positive tumor and/or HER2 low-expression tumor.

In the present application, the tumor may be CDK12-amplified tumor. For example, the tumor has relatively enhanced copies of CDK12 gene. For example, the tumor has both relatively enhanced copies of CDK12 gene and HER2 gene. For example, the CDK12-amplified tumor may comprise a solid tumor (for example, a breast cancer and/or a gastric cancer).

For example, the tumor may comprise a breast cancer and/or a gastric cancer. For example, the breast cancer may comprise HER2 positive breast cancer and/or HER2 low-expression breast cancer. For example, the breast cancer may comprise early breast cancer, locally advanced breast cancer and/or metastatic breast cancer. For example, the gastric cancer may comprise early gastric cancer, locally advanced gastric cancer and/or metastatic gastric cancer. For example, the subject in need thereof may have histologically or cytologically proven diagnosis of HER2-positive adenocarcinoma of the breast at the time of diagnosing locally advanced unresectable or metastatic disease.

For example, the HER2 inhibitor of present application may be administrated to the subject at a dose of about 15 mg/kg to about 35 mg/kg. For example, the dose of the HER2 inhibitor of present application may about 20 mg/kg to about 30 mg/kg. For example, the dose the HER2 inhibitor of present application may about 20 mg/kg. For example, the dose may about 30 mg/kg.

For example, the HER2 inhibitor of present application may be administrated about once every two weeks or about once every three weeks. For example, the dose may about 20 mg/kg, and the HER2 inhibitor of present application may be administered once every two weeks. For example, the dose may about 30 mg/kg, and the HER2 inhibitor of present application may be administered once every three weeks.

The HER2 inhibitor of present application may be administered by the same route of administration or by different routes of administration. For example, the HER2 inhibitor of present application may be administrated by intravenous administration.

In the present application, the method may comprise administrating the multiple CDK inhibitor of the present application. For example, the method may comprise administrating the HER2 inhibitor and the multiple CDK inhibitor of the present application.

In the present application, the medicinal product may comprise the multiple CDK inhibitor of the present application. For example, the medicinal product may comprise the HER2 inhibitor and the multiple CDK inhibitor of the present application.

For example, the medicinal product may comprise two separate packaged products of the HER2 inhibitor of present application and the multiple CDK inhibitor of present application, respectively.

In present application, the medicinal product to be administrated may be purchased for once, or may be purchased for several times. For example, one time for purchasing the HER2 inhibitor of present application, and another time for purchasing the multiple CDK inhibitor of present application. For another example, purchasing the HER2 inhibitor of present application and the multiple CDK inhibitor of present application together for one time.

For example, the medicinal product may comprise a pharmaceutical composition. For example, the pharmaceutical composition may comprise a container for containing the HER2 inhibitor of present application and the multiple CDK inhibitor of present application.

For example, the HER2 inhibitor and the multiple CDK inhibitor are not comprised in the same container. For example, the HER2 inhibitor and the multiple CDK inhibitor are comprised in separate containers, respectively.

In the present application, the medicinal product may be in a solid or liquid state.

In the present application, the multiple CDK inhibitor may be administrated by intravenous administration.

In the present application, the HER2 inhibitor may be administrated before, after or with the multiple CDK inhibitor.

In the present application, the method may comprise administrating the immune checkpoint inhibitor of the present application.

For example, the method may comprise administrating the HER2 inhibitor and the immune checkpoint inhibitor of the present application. For example, the method may comprise administrating the HER2 inhibitor, the multiple CDK inhibitor and the immune checkpoint inhibitor of the present application.

In the present application, the medicinal product may comprise the immune checkpoint inhibitor of the present application.

For example, the medicinal product may comprise the HER2 inhibitor and the immune checkpoint inhibitor of the present application. For example, the medicinal product may comprise the HER2 inhibitor, the multiple CDK inhibitor and the immune checkpoint inhibitor of the present application.

For example, the medicinal product may comprise two separate packaged products of the HER2 inhibitor of present application and the immune checkpoint inhibitor of present application, respectively.

For example, the medicinal product may comprise three separate packaged products of the HER2 inhibitor, the multiple CDK inhibitor and the immune checkpoint inhibitor of present application, respectively.

In present application, the medicinal product to be administrated may be purchased for once, or may be purchased for several times. For example, one time for purchasing the HER2 inhibitor of present application, another time for purchasing the immune checkpoint inhibitor of present application and/or another time for purchasing the multiple CDK inhibitor of present application. For example, purchasing the HER2 inhibitor of present application and the multiple CDK inhibitor of present application and the immune checkpoint inhibitor of present application together for one time.

For example, the medicinal product may comprise a pharmaceutical composition. For example, the pharmaceutical composition may comprise a container for containing the HER2 inhibitor of present application and a container for containing the immune checkpoint inhibitor of present application. For example, the pharmaceutical composition may comprise a container for containing the HER2 inhibitor, a container for containing the immune checkpoint inhibitor, and a container for containing the multiple CDK inhibitor.

For example, the pharmaceutical composition may comprise a container for containing the HER2 inhibitor of present application and the immune checkpoint inhibitor of present application. For example, the pharmaceutical composition may comprise a container for containing the HER2 inhibitor, the immune checkpoint inhibitor, and the multiple CDK inhibitor.

In the present application, the medicinal product may be in a solid or liquid state.

In the present application, the immune checkpoint inhibitor may be administrated by intravenous administration.

In the present application, the HER2 inhibitor may be administrated before, after or with the immune checkpoint inhibitor. In the present application, the multiple CDK inhibitor may be administrated before, after or with the immune checkpoint inhibitor. In the present application, the HER2 inhibitor may be administrated with the immune checkpoint inhibitor and the multiple CDK inhibitor.

For example, the HER2 inhibitor may be administrated to the subject at a dose of about 20 mg/kg to about 30 mg/kg. For example, the HER2 inhibitor may be administrated about once every two weeks or about once every three weeks. For example, the HER2 inhibitor may be administrated by intravenous administration.

For example, the immune checkpoint inhibitor may be administrated at dose of 0.01 mg/kg to 100 mg/kg. For example, the immune checkpoint inhibitor may be administrated about once every two weeks or about once every three weeks. For example, the immune checkpoint inhibitor may be administrated by intravenous administration.

For example, the HER2 inhibitor may be administrated before, after or with the immune checkpoint inhibitor. For example, the HER2 inhibitor may be administrated with the immune checkpoint inhibitor and the multiple CDK inhibitor.

In the present application, the method may comprise the following step: detecting the alteration in the subject in order to determine whether the subject is suitable for administrating the HER2 inhibitor of the present application.

For example, the detecting may be conducted through a sequencing of the HER2 protein in the subject. For example, the detecting may be conducted through a sequencing of the CDK12 gene in the subject. For example, the detecting may be conducted through a sequencing of the HER2 gene in the subject.

In the present application, the sequencing may comprise a NGS and/or a ddPCR. The NGS is next-generation sequencing, and may be used for determining the sequence of DNA or RNA in order to know the genetic variation, which may be related to the disease, for example, related to the tumor. The NGS may use DNA (for example, cDNA) as a sample. In the present application, the ddPCR is Droplet Digital PCR, which is a method for performing digital PCR basing on water-oil emulsion droplet technology. The ddPCR may be used to directly quantify and clonally amplify nucleic acids strands including DNA, cDNA, or RNA.

In the present application, the detecting may also be conducted by FISH and/or IHC. The FISH is Fluorescence in situ hybridization. The FISH may use fluorescent probes that bind to only those parts of a nucleic acid sequence with a high degree of sequence complementarity. The FISH may be used for detecting specific features (for example, the amplification, the mutation of the present application) in genome (including DNA and/or RNA). The IHC is Immunohistochemistry, and may be used for labeling of a protein (for example, HER2 and/or CDK12) in a tissue sample. The IHC may be very sensitive if the corresponding antibody is used for detecting the protein.

In the present application, the sequencing may use peripheral blood and/or tumor tissue from the subject in need as a sample. For example, the subject may be in need to preventing, alleviating or treating the tumor in present application. For example, the subject may be in need to determine whether the subject is suitable for administrating the HER2 inhibitor of the present application.

The present application also provides a use of the HER2 inhibitor of the present application, in combination with the multiple CDK inhibitor of the present application and/or the immune checkpoint inhibitor of the present application in the preparation of a medicament for alleviating or treating tumor or inhibiting tumor growth in a subject.

The present application also provides a use of the medicinal product of the present application in the preparation of a medicament for alleviating or treating tumor or inhibiting tumor growth in a subject.

Detecting Method and System Thereof

In another aspect, the present application provides a method of preventing, alleviating or treating cancer or inhibiting tumor growth in a subject, comprising: detecting the alteration of the present application in the subject, if the alteration exists, then the subject is suitable for administrating the HER2 inhibitor of the present application.

In another aspect, the present application provides a system for determining whether a subject is suitable for administrating the HER2 inhibitor of the present application, and the system comprises a detection module configured to detect the alteration of the present application in the subject.

For example, the detecting module may be configured to conducting sequencing of the protein and/or the gene encoding the protein of present application.

For example, the detecting module may be configured to conduct a sequencing of the HER2 gene. For example, the detecting module may be configured to conduct a sequencing of the CDK12 gene.

For example, the detecting module may be configured to conduct a sequencing of the HER2.

In present application, the detection module may comprise an agent for sequencing of said CDK12 gene and/or an agent for sequencing said HER2 gene. For example, the agent may comprise a primer of the HER2 gene, and/or a primer for the CDK12 gene. For example, the detection module may comprise agent for conducting NGS and/or ddPCR for HER2 gene and/or CDK12 gene.

In present application, the detection module may comprise an agent for sequencing HER2. For example, the agent may comprise a probe and/or an antibody for HER2. For example, the detection module may comprise agent for conducting FISH and/or IHC for HER2 and/or CDK12.

In present application, the system may comprise a sample collecting module.

For example, the sequencing may use a ctDNA from the subject as a sample. For example, the sequencing may use peripheral blood from the subject as a sample. For example, the sequencing may use tumor tissue from the subject as a sample.

For example, the sample collecting module may be configured to collect a ctDNA from the subject. For example, the sample collecting module may be configured to collect peripheral blood from the subject.

For example, the sample collecting module may comprise an agent for collecting and/or an agent for isolating ctDNA. For example, the sample collecting module may comprise a sterile needle, a tourniquet and/or a puncture site. For example, the sample collecting module may comprise a glass slide.

### EXAMPLES

The following examples are set forth so as to provide those of ordinary skill in the art with a complete disclosure and description of how to make and use the present application, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

CellTiter-Glo Luminescent Cell Viability Assay (CTG)

Cell Culture

SK-BR-3 (Human breast cancer, ATCC) were cultured in McCoy's 5a (Catalogue number: 12330-031, Gibco) plus 10% FBS (Catalogue number: FND500, lot number: 11G271, ExCell), AU565 (Human breast cancer, ATCC), HCC2218 (Human breast cancer, ATCC), HCC1954 (Human breast cancer, ATCC), KYSE-410 (Human esophageal cancer, DSMZ), NCI-H1781 (Human bronchioloalveolar carcinoma, ATCC), NCI-H2170 (Human squamous NSCLC, PUMC), NCI-N87 (Human gastric cancer, ATCC) were cultured in RPMI-1640 (Catalogue number: C22400500BT, lot number: 8118117, Gibco) plus 10% FBS. OE19 (Human esophageal cancer, DSMZ) were cultured in RPMI1640 plus 10% FBS and 2 mM L-Glutamine. EFM-192A (Human breast cancer. CoBioer) and ZR-75-30 (Human breast cancer, ATCC) were cultured in RPMI1640 plus 20% FBS. All cells were incubated at 37° C. 5% CO₂.

Drug Treatment

The HER2 inhibitor. Trastuzumab (Alphamab) and Pertuzumab (Alphamab) were all diluted with medium. The final concentrations of the HER2 inhibitor are 676.52, 169.13, 42.28, 10.57, 2.64, 0.88, 0.22, 0.055, 0.014 nM, The final concentrations of Trastuzumab are 687.13, 171.78, 42.95, 10.74, 2.68, 0.67, 0.17, 0.042, 0.01 nM, The final concentrations of Pertuzumab are 675.67, 168.92, 42.23, 10.56, 2.64, 0.66, 0.17, 0.041, 0.01 nM, Dinaciclib (Beyotime, SC6628), The final concentrations of Dinaciclib were 100, 33.33, 11.11, 3.70, 1.23, 0.41, 0.14, 0.046, 0.015 nM.

Procedures

Adjust cell density to the appropriate number with medium, add 90 μL cell suspensions (1500˜8000 cells) to each well of 96-well plates (Catalogue number: 3610, Corning). For combination treatment, 80 μL cell suspensions were used to seed. Then, Incubate the plates overnight in humidified incubator at 37° C. with 5% CO₂. Add 10 μL compound solution (10×) to each well (triplicates). Incubate the plates for incubator at 37° C. with 5% CO₂ for three days. After that, add 50 μL CellTiter-Glo® Reagent (Catalogue number: G7572, lot number: 0000309060, Promega) to each well. Mix contents for 2 minutes on an orbital shaker to facilitate cell lysis. Allow the plate to incubate at room temperature for 20 minutes to stabilize luminescent signal. Record luminescence using EnVision 2104 Multi Label Reader. The surviving rate (%) formula=(Luminesenceₛₐₘₚₗₑ−Luninesence_{medium control})/(Luminesence_{vehicle control}−Luminesence_{medium control})×100%. Use GraphPad Prism 5.0 software, display sigmoidal dose response-surviving rate graph by nonlinear regression model and calculate IC₅₀. The data also included the maximum inhibition rate.

The HER2 inhibitor of the present application has a common light chain and the first heavy chain and the second heavy chain, wherein the common light chain comprises an amino acid sequence as set forth in SEQ ID No.65: the variable region of the first heavy chain comprises an amino acid sequence as set forth in SEQ ID No.87 and the variable region of the second heavy chain comprises an amino acid sequence as set forth in SEQ ID No.88.

The dimer of the present application is a homodimer and each peptide of the dimer comprises an amino acid sequence of SEQ ID NO.40. The dimer of the present application comprises the ISVD specific for PD-L1 comprising an amino acid sequence as set forth in SEQ ID NO.6, and the ISVD specific for CLTA4 comprising an amino acid sequence as set forth in SEQ ID NO. 20.

### Example 1 Clinical Activity

Study Design and Patient Selection

This phase I, multicenter-center, open-label, 3+3, dose-escalation study was designed to evaluate the safety, tolerability, pharmacokinetics (PK), and preliminary antitumor activity, and identify the recommended phase II dose (RP2D) of the HER2 inhibitor in patients with HER2-positive metastatic breast cancer (MBC). The study protocol was approved by institutional review boards before patient recruitment and conducted in accordance with International Conference on Harmonization E6 Guidelines for Good Clinical Practice. Each patient provided signed informed consent before study enrollment.

Eligible patients were 18-75 years old and had histologically confirmed HER2 positive metastatic breast cancer. HER2 positivity status was determined according to ASCO/CAP 2018 guideline 5. Patients had received at least 1 prior line of anti-HER2 therapy in the metastatic setting, at least one measurable disease per RECIST 1.1 and baseline left-ventricular ejection fraction (LVEF)≥55%. Patients were excluded if they had unstable brain metastasis, malignant meningitis, had history of symptomatic interstitial lung disease, history of cumulative doxorubicin dose exceeding 300 mg/m² or equivalent or medically significant cardiac disorder. If patients had next-generation sequencing results from blood or archival tumor samples before informed consent was signed, the information would be collected.

In the 3+3, dose-escalation stage, patients received the HER2 inhibitor treatments with dosing consisted of 5 mg/kg QW, 10 mg/kg QW, 20 mg/kg Q2W, and 30 mg/kg Q3W. Eligible patients were given the HER2 inhibitor intravenously dose on the 21-day or 28-day cycle until disease progression, unacceptable toxicity, withdrawal of informed consent. Three patients were initially assigned to a starting dose level of 5 mg/kg. A dose-limiting toxicity (DLT) was defined as a toxic reaction related to study treatment after the administration that is unacceptable due to severity and/or irreversibility and limits further dose escalation. The DLT evaluation period was 28 days for dosing frequency of QW and Q2W, and 21 days for Q3W. Once an objective response (partial response or complete response) was observed at a certain dose level, the dose level will be expanded to enroll additional 23 to 25 patients to explore the efficacy, safety, and tolerability of the HER2 inhibitor. The highest dose level at which no more than one of six patients had a DLT was considered the maximum tolerated dose (MTD). Population pharmacokinetics and pharmacodynamics approach was used to determine RP2D if MTD cannot be identified.

Sample Size Determination and Statistical Analysis

The size of the dose-escalation cohort was based on a 3+3 phase I trial design. The sample size of the expansion cohorts was calculated using a one-sided accurate test method, with a presumption that each cohort in the dose expansion phase would enroll approximately 60 subjects. Assuming that the ORR in patients with HER2-positive MBC who failed the prior anti-HER2 treatments was 8-10%, a size of 60 patients can provide a power of 80% to detect an increase in ORR at a level of 30% (α=0.05). The safety analyses were based on the safety analysis set, efficacy analyses were based on the full analysis set (FAS) of efficacy.

Demographics, baseline characteristics, AEs, laboratory toxicities, and DLT were summarized using descriptive statistics. ORR (objective response rate), DCR (Disease Control Rate) and CBR (clinical benefit rate) were reported as point estimates and 95% exact binomial CIs using Clopper Pearson method. Survival outcomes including PFS and OS were estimated by the Kaplan-Meier method. For all PK endpoints, descriptive statistics and graphical display were conducted. The Tmax was described with median, 25 and 75 percentiles, minimum and maximum values. Statistical computation was performed with SAS 9.4.

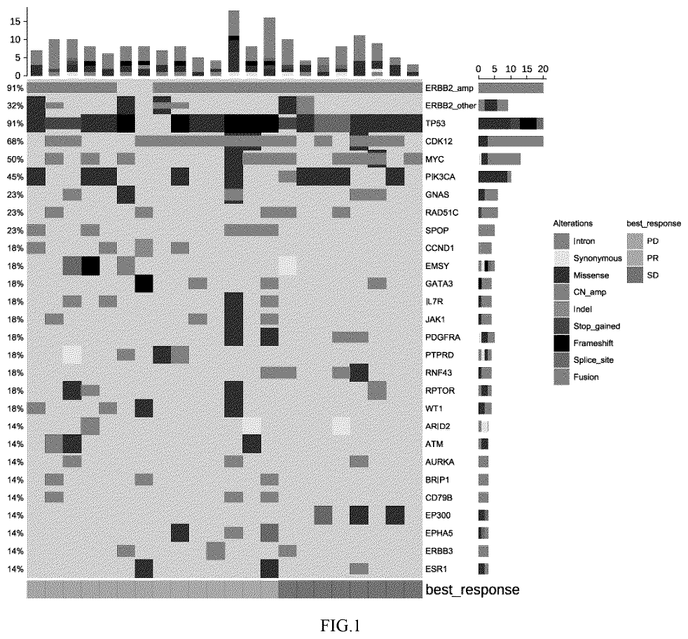

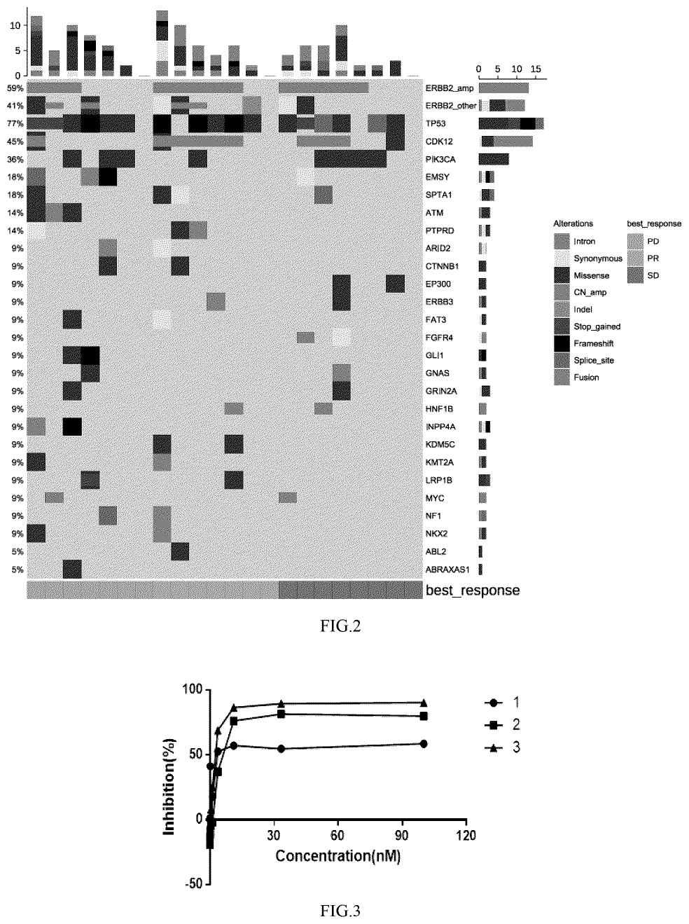

According to the baseline NGS information from tissue and peripheral blood ctDNA of 22 patients (FIG. 1 and FIG. 2), it is found gain-of-function HER2 mutations in 3 patient tissues (p.T862A, p.H878Y, p.R897W) and 2 patient ctDNA (p.T862A and p.H878Y, which was consistent with the corresponding tissue NGS information). The best responses of these 3 patients were 2 PD and 1 size-increased SD. TP53, CDK12, MYC and PIK3CA were the 4 most frequently altered genes detected in the tissue of the 22 patients. However. MYC alteration was not common in the ctDNA analysis. The only 2 patients without HER2 amplification detected in tissues had the best response of PD. All patients with responses to the HER2 inhibitor had CDK12 amplification. Those patients with PIK3CA mutation detected in tissue or ctDNA were relatively insensitive to the HER2 inhibitor.

Exploratory study demonstrated that the best responses of 3 patients with gain-of-function HER2 mutations were 2 PD and 1 size-increased SD. Actually, HER2 p.T862A, p.H878Y, p.R897W mutations all lies within the HER2 protein kinase domain. In some preclinical study, transformed cells expressing HER2 gain-of-function mutations demonstrated resistance to trastuzumab. TP53, CDK12, and PIK3CA were among the most frequently altered genes detected in both tissue and ctDNA. Interestingly, all PR patients had CDK12 amplification. Further study of the role of CDK12 in HER2-positive MBC will help to better understand the biological characteristics of this kind of patients and discover potential treatments for patients with concomitant CDK12 amplifications. Patients with PIK3CA mutation respond relatively less to the HER2 inhibitor, which is consistent with previous studies showing that mutations in PIK3CA may play a very important role in the trastuzumab/pertuzumab resistance and progression of MBC.

### Example 2 Tumor Inhibition

The HER2 inhibitor alone or in combination with Dinaciclib was used to inhibit the proliferation of the Herceptin-resistant BT474 cells (HER2-positive breast cancer cells). Herceptin-resistant BT474 cells was obtained from a subject who had been administrated Herceptin for at least one year and showing drug-resistance to Herceptin.

Then the Herceptin-resistant BT474 cells were be treated with the HER2 inhibitor alone or in combination with Dinaciclib on different concentration, and the cells were cultured for 3 days. The CCK-8 method was used to measure the OD450 value.

The results can be seen in FIG. 3. In FIG. 3, plots 1-3 are corresponding to the result of treating with the HER2 inhibitor alone, with Dinaciclib alone and with the combination of the HER2 inhibitor and Dinaciclib, respectively. From the results in FIG. 3, it is shown that the maximum inhibition rate of the combination of the HER2 inhibitor and Dinaciclib was about 90% while the maximum inhibition rate of the HER2 inhibitor alone is less than 60%. Hence, the combination of the HER2 inhibitor and Dinaciclib has a significantly better proliferation inhibition effect on Herceptin-resistant BT474 cells than the HER2 inhibitor alone or Dinaciclib alone.

The HER2 inhibitor alone or in combination with Dinaciclib was used to inhibit the proliferation of the Herceptin-resistant N87 cells (HER2-positive gastric cancer cells). Herceptin-resistant N87 cells was obtained from a subject who had been administrated Herceptin for at least one year and showing drug-resistance to Herceptin.

Then the Herceptin-resistant N87 cells were be treated with the HER2 inhibitor alone or in combination with Dinaciclib on different concentration, and the cells were cultured for 3 days. The CCK-8 method was used to measure the OD450 value.

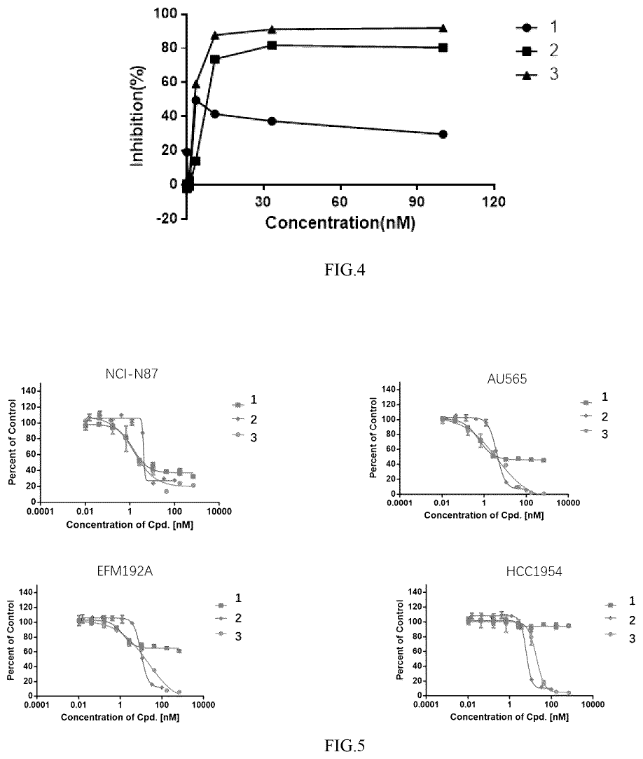

The results can be seen in FIG. 4. In FIG. 4, plots 1-3 are corresponding to the result of treating with the HER2 inhibitor alone, with Dinaciclib alone and with the combination of the HER2 inhibitor and Dinaciclib, respectively. From the results in FIG. 4, it is shown that the maximum inhibition rate of the combination of the HER2 inhibitor and Dinaciclib was about 90% while the maximum inhibition rate of the HER2 inhibitor alone is less than 40%. Hence, the combination of the HER2 inhibitor and Dinaciclib has a significantly better proliferation inhibition effect on Herceptin-resistant N87 cells than the HER2 inhibitor alone or Dinaciclib alone.

### Example 3 the Tumor Inhibition by the Combination of the HER2 Inhibitor and Dinaciclib

The example 3 was conducted according to the procedures of CellTiter-Glo® Luminescent Cell Viability Assay (CTG).

The results can be seen in FIGS. 5-6. In FIGS. 5-6, plots 1-3 are corresponding to the result of treating with the HER2 inhibitor alone, with Dinaciclib alone and with the combination of the HER2 inhibitor and Dinaciclib, respectively.

From the results in FIGS. 5-6, it is shown that the combination of the HER2 inhibitor and Dinaciclib (especially at a relative higher concentrations) has a significantly better proliferation inhibition effect on CDK12-amplified solid tumor cells than the HER2 inhibitor alone; and has a slightly better proliferation inhibition effect than that of Dinaciclib alone.

### Example 4 the Tumor Inhibition by the Combination of the HER2 Inhibitor and Dinaciclib

The example 4 was conducted according to the procedures of CellTiter-Glo® Luminescent Cell Viability Assay (CTG).

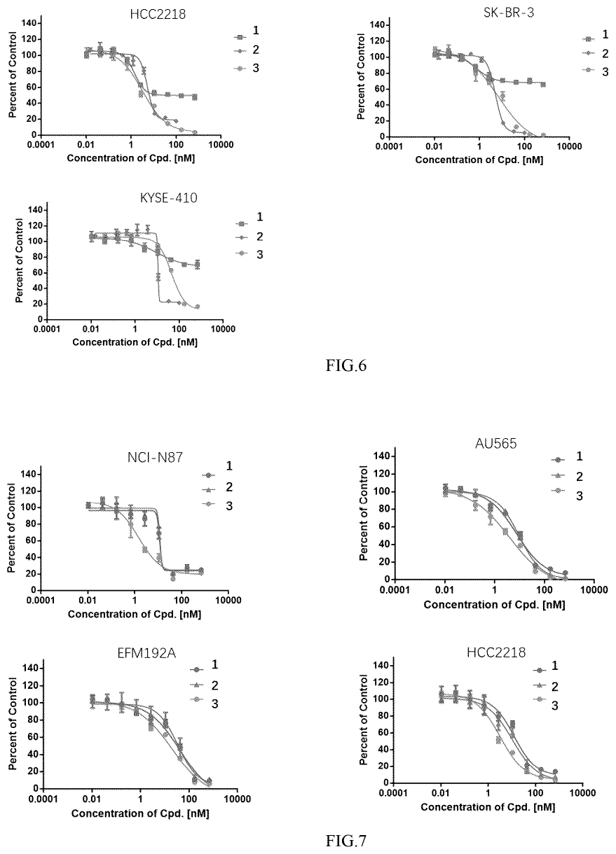

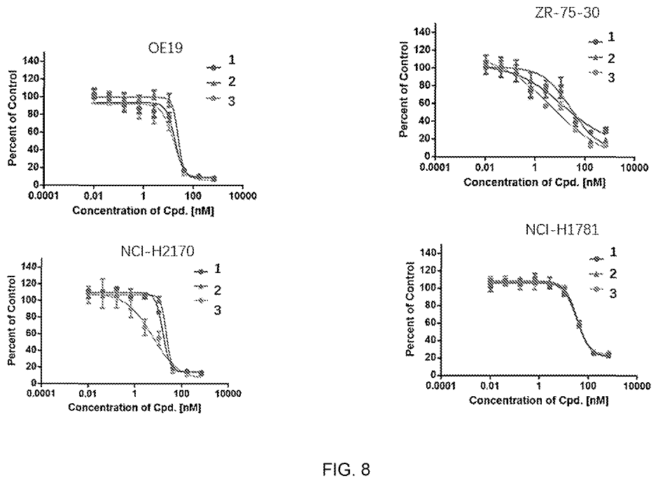

The results can be seen in FIGS. 7-8. In FIGS. 7-8, plots 1-3 are corresponding to the result of treating with the combination of Trastuzumab and Dinaciclib, with the combination of Trastuzumab, Pertuzumab and Dinaciclib, and with the combination of the HER2 inhibitor and Dinaciclib, respectively.

From the results in FIGS. 7-8, it is shown that the combination of the HER2 inhibitor and Dinaciclib has a significantly better proliferation inhibition effect on CDK12-amplified solid tumor cells than the combination of Trastuzumab, Pertuzumab and Dinaciclib; and has a relatively similar proliferation inhibition effect than that of the combination of Trastuzumab. Pertuzumab and Dinaciclib on partially CDK12-unamplified solid tumor cells (such as OE19 cell, NCI-H1781 cell, ZR-75-30 cell and NCI-H2170 cell).

### Example 5 the Tumor Inhibition on Gastric Tumor Cell

The example 5 was conducted according to the procedures of CellTiter-Glo® Luminescent Cell Viability Assay (CTG).

The results can be seen in FIG. 9. In FIG. 9, plots 1-2 are corresponding to the result of treating with Trastuzumab alone and with the HER2 inhibitor alone, respectively.

From the results in FIG. 9, it is shown that the HER2 inhibitor has a significantly better proliferation inhibition effect on gastric tumor cells (NCI-N87) than the Trastuzumab. And NCI-N87 cells express both HER2 and CDK12.

### Example 6 the Tumor Inhibition on Tumor Cell

The example 6 was conducted according to the procedures of CellTiter-Glo® Luminescent Cell Viability Assay (CTG).

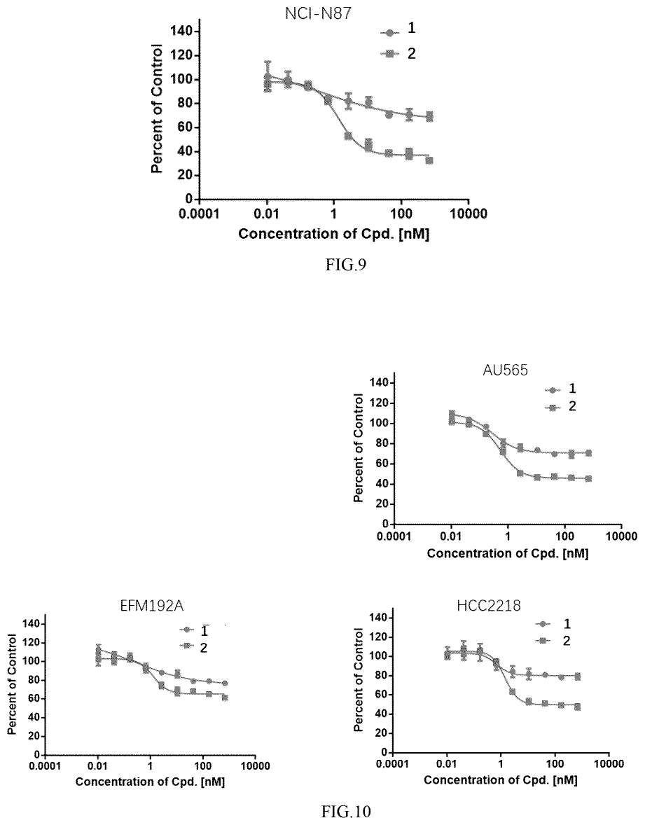

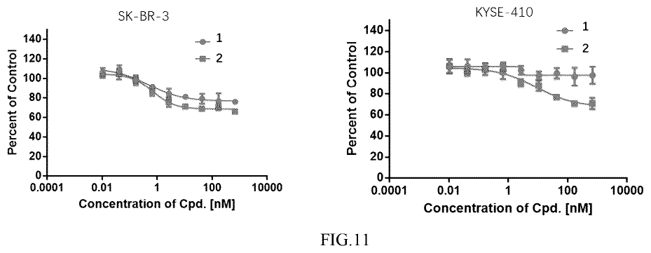

The results can be seen in FIGS. 10-11. In FIGS. 10-11, plots 1-2 are corresponding to the result of treating with Trastuzumab alone and with the HER2 inhibitor alone, respectively.

From the results in FIGS. 10-11, it is shown that the HER2 inhibitor has a significantly better proliferation inhibition effect on CDK12-amplified solid tumor cells than the Trastuzumab.

### Example 7 CDK12 is a Biomarker for Better Response of the HER2 Inhibitor

To explore the molecular mechanisms underlying patients' differential responses to the HER2 inhibitor and to identify promising biomarkers. 22 HER2′ patients were recruited based on immunohistochemistry (IHC) test (Table 2). A NGS panel-based gene test was performed on patient tissue samples and the gene test confirmed that 20 patients had HER2 amplification, while 14 of those same patients had CDK12 co-amplification. Of note, responders were highly enriched with HER2/CDK12 co-amplification (ORR of 50% vs 0%. Fisher's exact test, P=0.05) and achieved longer PFS (8.2 vs 2.7 months. Log-rank test, P=0.04), suggesting that HER2/CDK12 co-amplification contributes to more effective clinical outcomes and this feature can be potentially used as a biomarker for the HER2 inhibitor treatment. Using multi-omics data of The Cancer Genome Atlas (TCGA), HER2/CDK12 co-amplifications were shown to be widespread across a broad range of cancer types, especially in kidney renal papillary cell carcinoma (KIRP) and lung squamous cell carcinoma (LUAD). Approximately 30% of patients with breast cancer (ranked as the 9^{th} most prevalent cancer among the 33 cancer types) exhibited HER2/CDK12 co-amplification, which increased to approximately 70% in HER2-enriched subtype defined by PAM50 in breast cancer. Since 80% of the patients with HER2⁺ subtype breast cancer exhibited HER2 amplification, this subtype was made the focus in this study. It was found that loss-of-function mutations of well-known driver genes in breast cancer, PTEN, were significantly enriched in this cancer subtype without HER2/CDK12 co-amplification (Fisher's exact test, P=1.3×10⁻³). As expected, HER2/CDK12 co-amplification resulted in significantly higher expression levels of the two genes at the mRNA level. In addition, compared with the co-amplification-free group, the HER2/CDK12 co-amplification group showed significantly lower activities for cell cycle, mTOR, and DNA repair pathways based on both mRNA and protein expression levels. In addition, the co-amplification group showed lower immune activities and less abundance of immune cells, including CD8⁺ T cells (P=1.7×10⁻²), neutrophils (P=1.9×10⁻²), and B cells (P=2.9×10⁻²). Taken together, breast cancer patients with HER2/CDK12 co-amplification have distinct altered molecular profiles, dysregulated pathways, and tumor microenvironments, which may be responsible for the observed differential response to the HER2 inhibitor of the present application. These results suggest that HER2/CDK12 co-amplification may be a potential predictive biomarker for the HER2 inhibitor of the present application treatment response.

Using CellTiter-Glo® Luminescent Cell Viability Assay (CTG), the HER2 inhibitor of the present application was found to be more effective and potent than trastuzumab+pertuzumab in trastuzumab resistant cell lines with respect to potency and efficacy parameters such as absolute EC₅₀, relative EC₉₅, and maximum inhibition rate. Consistent with these findings, the HER2 inhibitor of the present application was shown to be more effective and potent against HER2/CDK12 co-amplified cell lines than in HER2 amplified/CDK12 non-amplified cell lines. The maximum inhibition rates of the 3 combinations including dinaciclib (trastuzumab+dinaciclib, trastuzumab+pertuzumab+dinaciclib, the HER2 inhibitor+dinaciclib) were all very high, and no significant difference of inhibition effect between CDK12 co-amplified and no-co-amplified cell lines were observed.

TABLE 2
Clinical information for the patients used for gene test in this study

| | | | Best Overall | | | | |
|---|---|---|---|---|---|---|---|
| Patient_ID | Age | Stage | response | PFS status | PFS (months) | HER2 | CDK12 |
| P01 | 46 | NA | SD | 0 | 4.50 | Y | Y |
| P02 | 56 | NA | SD | 0 | 18.73 | Y | Y |
| P03 | 56 | IV | PD | 0 | 2.69 | Y | N |
| P04 | 57 | IV | SD | 0 | 4.07 | Y | Y |
| P05 | 56 | IV | PD | 0 | 1.31 | Y | N |
| P06 | 43 | IV | PD | 0 | 1.31 | Y | Y |
| P07 | 67 | NA | SD | 0 | 5.52 | Y | Y |
| P08 | 54 | NA | PD | 0 | 1.28 | N | Y |
| P09 | 63 | III | PR | 0 | 8.38 | Y | Y |
| P10 | 55 | IV | SD | 1 | 2.66 | Y | N |
| P11 | 62 | IV | PR | 0 | 6.77 | Y | Y |
| P12 | 41 | IV | PR | 1 | 8.18 | Y | Y |
| P13 | 44 | NA | PD | 0 | 2.69 | Y | N |
| P14 | 47 | NA | PD | 0 | 1.25 | Y | Y |
| P15 | 60 | NA | SD | 1 | 8.15 | Y | Y |
| P16 | 31 | IV | PR | 0 | 8.18 | Y | Y |
| P17 | 36 | IV | SD | 0 | 5.45 | Y | N |
| P18 | 37 | NA | PR | 1 | 8.21 | Y | Y |
| P19 | 48 | NA | PD | 0 | 1.48 | N | N |
| P20 | 58 | NA | PR | 1 | 6.64 | Y | Y |
| P21 | 30 | NA | SD | 0 | 5.59 | Y | N |
| P22 | 64 | IV | PR | 1 | 5.49 | Y | Y |

Abbreviations:
NA, not available;
PD, progressive disease;
PFS, progression-free survival;
PR, partial response;
SD, stable disease

### Example 8 Study of the HER2 Inhibitor in Combination with the Dimer of the Present Disclosure in Subjects with CDK12 Positive Tumor

This study is to evaluate the safety and efficacy of the HER2 inhibitor in combination with the dimer of the present disclosure in subjects with CDK12 positive tumor.

The dose group (the Her2 inhibitor 15-35 mg/kg Q2W+the dimer 1-5 mg/kg Q2W) engaged 6 subjects.

The results show that the HER2 inhibitor in combination with the dimer of the present disclosure treated the CDK12 positive tumor effectively in 4 subjects.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of preventing, alleviating or treating tumor or inhibiting tumor growth in a subject, comprising: administrating to the subject a HER2 inhibitor,
wherein said subject comprises an alteration in a protein and/or a gene encoding said protein, and said protein comprises HER2 and/or CDK12; and/or, wherein said tumor is CDK12-amplified tumor,
wherein said HER2 inhibitor is a bispecific antibody or an antigen binding portion thereof, and is capable of binding to different epitopes of human HER2,
wherein said alteration comprises at least one mutation of said HER2 protein, wherein said mutation comprises T862A, H878Y and/or R897W; and/or, wherein said alteration comprises a co-amplification of CDK12 gene and HER2 gene.

2. The method according to claim 1, wherein said HER2 inhibitor is a bispecific antibody or the antigen binding portion thereof, and said bispecific antibody or the antigen binding portion thereof has a first heavy chain and a second heavy chain, and wherein said first heavy chain and said second heavy chain are capable of correctly assembling with said light chains respectively under physiological conditions or during in vitro protein expression,
wherein variable region of said first light chain and/or said second light chain comprises an amino acid sequence as set forth in any one of SEQ ID NO: 91-96,
wherein said first light chain comprises an amino acid sequence as set forth in any one of SEQ ID NO: 65-70, and/or, said second light chain comprises an amino acid sequence as set forth in any one of SEQ ID NO: 65-70,
wherein variable region of said first heavy chain comprises an amino acid sequence as set forth in SEQ ID NO: 87; and variable region of said second heavy chain comprises an amino acid sequence as set forth in SEQ ID NO: 88, and/or,
wherein two heavy chains thereof comprise a sequence as set forth in any one of SEQ ID NO: 80-81, 83-84, 97-100.

3. The method according to claim 1, wherein said HER2 inhibitor is administrated to the subject at a dose of about 15 mg/kg to about 35 mg/kg.

4. The method according to claim 1, wherein said HER2 inhibitor is administrated about once every two weeks or about once every three weeks.

5. The method according to claim 1, wherein said dose of said HER2 inhibitor is about 20 mg/kg, and said HER2 inhibitor is administered once every two weeks, and/or, said dose of said HER2 inhibitor is about 30 mg/kg, and said HER2 inhibitor is administered once every three weeks.

6. The method according to claim 1, wherein said alteration comprises a mutation of said HER2 protein, wherein said mutation comprises T862A, H878Y and R897W.

7. The method according to claim 1, wherein said subject was not responsive to a conventional therapy for HER2-related tumor, said conventional therapy for HER2-related tumor comprises administrating HER2-ADC, pyrotinib, neratinib, tucatinib, trastuzumab and/or pertuzumab, and/or, said conventional therapy for HER2-related tumor comprises administrating docetaxel, capecitabine and/or lapatinib.

8. The method according to claim 1, wherein said tumor comprises solid tumor, and/or, wherein said tumor comprises HER2 positive tumor and/or HER2 low-expression tumor.

9. The method according to claim 1, wherein said tumor comprises a breast cancer and/or a gastric cancer, said breast cancer comprises HER2 positive breast cancer and/or HER2 low-expression breast cancer, said breast cancer comprises early breast cancer, locally advanced breast cancer and/or metastatic breast cancer; and/or said gastric cancer comprises early gastric cancer, locally advanced gastric cancer and/or metastatic gastric cancer.

10. The method according to claim 1, wherein said method comprises a following step: detecting said alteration in said subject in order to determine whether said subject is suitable for administrating said HER2 inhibitor, said detecting comprising conducting a sequencing of said HER2 protein in said subject, said detecting comprising conducting a sequencing of said CDK12 gene and/or, said detecting comprising conducting a sequencing of said HER2 gene.

11. The method according to claim 1, wherein said method further comprises administrating a multiple CDK inhibitor, wherein said multiple CDK inhibitor inhibits CDK12.

12. The method according to claim 11, wherein said multiple CDK inhibitor is selected from a group consisting of: THZ531, Dinaciclib and SR-3029.

13. The method according to claim 1, wherein said method further comprises administrating an immune checkpoint inhibitor, wherein said immune checkpoint inhibitor is capable of specifically binding to PD-L1 and CTLA4, wherein said immune checkpoint inhibitor is a dimer, and said dimer is formed by two polypeptide chains, with each of said two polypeptide chains comprising an antibody Fc subunit, wherein said dimer comprises two or more immunoglobulin single variable domains (ISVDs), at least one of said ISVDs is specific for PD-L1, and at least one of said ISVDs is specific for CTLA4, and/or, wherein for one or both of said two polypeptide chains: said ISVD specific for PD-L1 is fused to said ISVD specific for CTLA4, optionally via a linker; and said ISVD specific for PD-L1 is fused to said antibody Fc subunit, optionally via a linker; and/or,
said ISVD specific for PD-L1 comprises a heavy chain CDR3 comprising an amino acid sequence as set forth in SEQ ID NO: 1; optionally, said ISVD specific for PD-L1 comprises a heavy chain CDR3 comprising an amino acid sequence as set forth in any one of SEQ ID NO: 5 and 9; and/or,
said ISVD specific for PD-L1 comprises a heavy chain CDR1 comprising an amino acid sequence as set forth in SEQ ID NO: 2; optionally, said ISVD specific for PD-L1 comprises a heavy chain CDR1 comprising an amino acid sequence as set forth in any one of SEQ ID NO: 3 and 7; and/or,
said ISVD specific for PD-L1 comprises a heavy chain CDR2 comprising an amino acid sequence as set forth in any one of SEQ ID NO: 4, 8 and 11; and/or,
said ISVD specific for CTLA4 comprises a heavy chain CDR3 comprising an amino acid sequence as set forth in SEQ ID NO: 19; and/or,
said ISVD specific for CTLA4 comprises a heavy chain CDR1 comprising an amino acid sequence as set forth in SEQ ID NO: 17; and/or,
said ISVD specific for CTLA4 comprises a heavy chain CDR2 comprising an amino acid sequence as set forth in SEQ ID NO: 16; said ISVD specific for CTLA4 comprises a heavy chain CDR2 comprising an amino acid sequence as set forth in any one of SEQ ID NO: 18, 21 and 23; and/or,
wherein said ISVD specific for PD-L1 comprises a heavy chain variable domain comprising an amino acid sequence as set forth in any one of SEQ ID NO: 6, 10, 12, 13, 14 and 15; and/or, wherein said ISVD specific for CTLA4 comprises a heavy chain variable domain comprising an amino acid sequence as set forth in any one of SEQ ID NO: 20, 22, and 24-32.

14. The method according to claim 13, wherein one or both of said two polypeptide chains comprises an amino acid sequence as set forth in any one of SEQ ID NO: 40-43, 46, 48 and 50.

15. A medicinal product comprising: a HER2 inhibitor and a multiple CDK inhibitor,
wherein said HER2 inhibitor is a bispecific antibody or the antigen binding portion thereof, and said bispecific antibody or the antigen binding portion thereof has a first heavy chain and a second heavy chain, and wherein said first heavy chain and said second heavy chain are capable of correctly assembling with said light chains respectively under physiological conditions or during in vitro protein expression,
wherein variable region of said first light chain and/or said second light chain comprises an amino acid sequence as set forth in any one of SEQ ID NO: 91-96,
wherein said first light chain comprises an amino acid sequence as set forth in any one of SEQ ID NO: 65-70, and/or, said second light chain comprises an amino acid sequence as set forth in any one of SEQ ID NO: 65-70,
wherein variable region of said first heavy chain comprises an amino acid sequence as set forth in SEQ ID NO: 87; and variable region of said second heavy chain comprises an amino acid sequence as set forth in SEQ ID NO: 88, and/or,
wherein two heavy chains thereof comprise a sequence as set forth in any one of SEQ ID NO: 80-81, 83-84, 97-100; and/or, said multiple CDK inhibitor is selected from a group consisting of: THZ531, Dinaciclib and SR-3029.

16. The medicinal product according to claim 15, wherein said medicinal product further comprises an immune checkpoint inhibitor, wherein said immune checkpoint inhibitor is capable of specifically binding to PD-L1 and CTLA4, wherein said immune checkpoint inhibitor is a dimer, and said dimer is formed by two polypeptide chains, with each of said two polypeptide chains comprising an antibody Fc subunit, wherein said dimer comprises two or more immunoglobulin single variable domains (ISVDs), at least one of said ISVDs is specific for PD-L1, and at least one of said ISVDs is specific for CTLA4, and/or, wherein for one or both of said two polypeptide chains: said ISVD specific for PD-L1 is fused to said ISVD specific for CTLA4, optionally via a linker; and said ISVD specific for PD-L1 is fused to said antibody Fc subunit, optionally via a linker; and/or,
said ISVD specific for PD-L1 comprises a heavy chain CDR3 comprising an amino acid sequence as set forth in SEQ ID NO: 1; optionally, said ISVD specific for PD-L1 comprises a heavy chain CDR3 comprising an amino acid sequence as set forth in any one of SEQ ID NO: 5 and 9; and/or,
said ISVD specific for PD-L1 comprises a heavy chain CDR1 comprising an amino acid sequence as set forth in SEQ ID NO: 2; optionally, said ISVD specific for PD-L1 comprises a heavy chain CDR1 comprising an amino acid sequence as set forth in any one of SEQ ID NO: 3 and 7; and/or,
said ISVD specific for PD-L1 comprises a heavy chain CDR2 comprising an amino acid sequence as set forth in any one of SEQ ID NO: 4, 8 and 11; and/or,
said ISVD specific for CTLA4 comprises a heavy chain CDR3 comprising an amino acid sequence as set forth in SEQ ID NO: 19; and/or,
said ISVD specific for CTLA4 comprises a heavy chain CDR1 comprising an amino acid sequence as set forth in SEQ ID NO: 17; and/or,
said ISVD specific for CTLA4 comprises a heavy chain CDR2 comprising an amino acid sequence as set forth in SEQ ID NO: 16; said ISVD specific for CTLA4 comprises a heavy chain CDR2 comprising an amino acid sequence as set forth in any one of SEQ ID NO: 18, 21 and 23; and/or,
wherein said ISVD specific for PD-L1 comprises a heavy chain variable domain comprising an amino acid sequence as set forth in any one of SEQ ID NO: 6, 10, 12, 13, 14 and 15; and/or, wherein said ISVD specific for CTLA4 comprises a heavy chain variable domain comprising an amino acid sequence as set forth in any one of SEQ ID NO: 20, 22, and 24-32.

17. The medicinal product according to claim 16, wherein one or both of said two polypeptide chains comprises an amino acid sequence as set forth in any one of SEQ ID NO: 40-43, 46, 48 and 50.

18. A method of determining whether a subject is suitable for administrating a HER2 inhibitor, comprising: detecting an alteration in said subject, if said alteration exists, said subject is suitable for administrating said HER2 inhibitor, wherein said alteration is in a protein and/or a gene encoding said protein, and said protein comprises HER2 and/or CDK12,
wherein said HER2 inhibitor is a bispecific antibody or the antigen binding portion thereof, and said bispecific antibody or the antigen binding portion thereof has a first heavy chain and a second heavy chain, and wherein said first heavy chain and said second heavy chain are capable of correctly assembling with said light chains respectively under physiological conditions or during in vitro protein expression,
wherein variable region of said first light chain and/or said second light chain comprises an amino acid sequence as set forth in any one of SEQ ID NO: 91-96,
wherein said first light chain comprises an amino acid sequence as set forth in any one of SEQ ID NO: 65-70, and/or, said second light chain comprises an amino acid sequence as set forth in any one of SEQ ID NO: 65-70,
wherein variable region of said first heavy chain comprises an amino acid sequence as set forth in SEQ ID NO: 87; and variable region of said second heavy chain comprises an amino acid sequence as set forth in SEQ ID NO: 88,
wherein said alteration comprises at least one mutation of said HER2 protein, wherein said mutation comprises T862A, H878Y and/or R897W; and/or, wherein said alteration comprises a co-amplification of CDK12 gene and HER2 gene.

19. The method according to claim 18, wherein said detecting comprising conducting a sequencing of said HER2 protein in said subject, said detecting comprising conducting a sequencing of said CDK12 gene, and/or, said detecting comprising conducting a sequencing of said HER2 gene.

20. The method according to claim 19, wherein said sequencing comprises a NGS, and/or a ddPCR; and/or, said sequencing uses a ctDNA from said subject.


SEQUENCE LISTING (104 sequences)

SEQ ID NO: 1 - protein, 19 aa, synthetic construct
  REGION 1..19: note = CDR3 of antiPD-L1
  VARIANT 4: note = X = E or G
  VARIANT 5: note = X = D or Y
  VARIANT 9: note = X = T or P
  VARIANT 10: note = X = L or G
  VARIANT 11: note = X = V or P
  VARIANT 12: note = X = T or A
DSFXXPTCXXXXSSGAFQY

SEQ ID NO: 2 - protein, 10 aa, synthetic construct
  REGION 1..10: note = CDR1 of antiPD-L1
  VARIANT 2: note = X = K or N
  VARIANT 3: note = X = M or I
  VARIANT 4: note = X = S or I
  VARIANT 5: note = X = S or R
  VARIANT 6: note = X = R or V
GXXXXXRCMA

SEQ ID NO: 3 - protein, 10 aa, synthetic construct
  REGION 1..10: note = PD-L1 ISVD-9 CDR1
GKMSSRRCMA

SEQ ID NO: 4 - protein, 17 aa, synthetic construct
  REGION 1..17: note = PD-L1 ISVD-9 CDR2
KLLTTSGSTYLADSVKG

SEQ ID NO: 5 - protein, 19 aa, synthetic construct
  REGION 1..19: note = PD-L1 ISVD-9 CDR3
DSFEDPTCTLVTSSGAFQY

SEQ ID NO: 6 - protein, 128 aa, synthetic construct
  REGION 1..128: note = PD-L1 ISVD-9
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSS

SEQ ID NO: 7 - protein, 10 aa, synthetic construct
  REGION 1..10: note = PD-L1 ISVD-6 CDR1
GNIIRVRCMA

SEQ ID NO: 8 - protein, 17 aa, synthetic construct
  REGION 1..17: note = PD-L1 ISVD-6 CDR2
NILTTTISTYLADSVKG

SEQ ID NO: 9 - protein, 19 aa, synthetic construct
  REGION 1..19: note = PD-L1 ISVD-6 CDR3
DSFGYPTCPGPASSGAFQY

SEQ ID NO: 10 - protein, 128 aa, synthetic construct
  REGION 1..128: note = PD-L1 ISVD-6
QVQLQESGGGSVQAGGSLRLSCAASGNIIRVRCMAWFRQAPGKERERGPNILTTTISTYLADSVKGRFTISQNNAKSTVYLQMNSLKPEDTAMYYCAADSFGYPTCPGPASSGAFQYWGQGTQVTVSS

SEQ ID NO: 11 - protein, 17 aa, synthetic construct
  REGION 1..17: note = PD-L1 ISVD-m3 CDR2
DVNPNSGGSIYNQRFKG

SEQ ID NO: 12 - protein, 128 aa, synthetic construct
  REGION 1..128: note = PD-L1 ISVD-m3
QVQLQESGGGLVQPGGSLRLSCAASGKMSSRRCMAWFRQAPGKERERVADVNPNSGGSIYNQRFKGRFTISQNNAKSTVYLQMNSLKPEDTAMYYCAADSFEDPTCTLVTSSGAFQYWGQGTQVTVSS

SEQ ID NO: 13 - protein, 128 aa, synthetic construct
  REGION 1..128: note = PD-L1 ISVD-4
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 14 - protein, 128 aa, synthetic construct
  REGION 1..128: note = PD-L1 ISVD-11
QVQLVESGGGLVQPGGSLRLSCAASGKMSSRRCMAWFRQAPGKGLERVAKLLTTSGSTYLADSVKGRFTISRDNSKNTVYLQMNSLRAEDTAVYYCAADSFEDPTCTLVTSSGAFQYWGQGTLVTVSS

SEQ ID NO: 15 - protein, 128 aa, synthetic construct
  REGION 1..128: note = PD-L1 ISVD-13
QVQLVESGGGLVQPGGSLRLSCAASGKMSSRRCMAWFRQAPGKERERVAKLLTTSGSTYLADSVKGRFTISRDNSKNTVYLQMNSLKAEDTAVYYCAADSFEDPTCTLVTSSGAFQYWGQGTLVTVSS

SEQ ID NO: 16 - protein, 17 aa, synthetic construct
  REGION 1..17: note = CDR2 of CTLA4
  VARIANT 3: note = X = Y or S
  VARIANT 4: note = X = I or L
AIXXGGGSTYYADSVKG

SEQ ID NO: 17 - protein, 5 aa, synthetic construct
  REGION 1..5: note = CTLA4 ISVD-34 CDR1
AYCMG

SEQ ID NO: 18 - protein, 17 aa, synthetic construct
  REGION 1..17: note = CTLA4 ISVD-34 CDR2
AIYIGGGSTYYADSVKG

SEQ ID NO: 19 - protein, 19 aa, synthetic construct
  REGION 1..19: note = CTLA4 ISVD-34 CDR3
DVIPTETCLGGSWSGPFGY

SEQ ID NO: 20 - protein, 128 aa, synthetic construct
  REGION 1..128: note = CTLA4 ISVD-34
QVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSS

SEQ ID NO: 21 - protein, 17 aa, synthetic construct
  REGION 1..17: note = CTLA4 ISVD-C1 CDR2
AIYLGGGSTYYADSVKG

SEQ ID NO: 22 - protein, 128 aa, synthetic construct
  REGION 1..128: note = CTLA4 ISVD-C1
QVQLQESGGGSVQAGGSLGLSCAASGYIYSAYCMGWFRQAPGKEREGVAAIYLGGGSTYYADSVKGRFTISQDIAKNTLYLQMNSLKPEDTAMYSCAADVIPTETCLGGSWSGPFGYWGQGTQVTVSS

SEQ ID NO: 23 - protein, 17 aa, synthetic construct
  REGION 1..17: note = CTLA4 ISVD-13 CDR2
AISIGGGSTYYADSVKG

SEQ ID NO: 24 - protein, 128 aa, synthetic construct
  REGION 1..128: note = CTLA4 ISVD-13
QVQLQESGGGSVQAGGSLRLSCAASGYIYSAYCMGWFRQAPGKERVGVAAISIGGGSTYYADSVKGRFTISQDNAKNTLYLQMNSLKPEDTAMYYCAADVIPTETCLGGSWSGPFGYWGQGTQVTVSS

SEQ ID NO: 25 - protein, 128 aa, synthetic construct
  REGION 1..128: note = CTLA4 ISVD-26
QVQLVESGGGLVQPGGSLRLSCAASGYIYSAYCMGWFRQAPGKERVGVAAISIGGGSTYYADSVKGRFTISQDNSKNTLYLQMNSLRAEDTAVYYCAADVIPTETCLGGSWSGPFGYWGQGTLVTVSS

SEQ ID NO: 26 - protein, 128 aa, synthetic construct
  REGION 1..128: note = CTLA4 ISVD-27
QVQLVESGGGLVQPGGSLRLSCAASGYIYSAYCMGWFRQAPGKERVGVAAISIGGGSTYYADSVKGRFTISRDNSKNTLYLQMNSLRAEDTAVYYCAADVIPTETCLGGSWSGPFGYWGQGTLVTVSS

SEQ ID NO: 27 - protein, 128 aa, synthetic construct
  REGION 1..128: note = CTLA4 ISVD-28
QVQLVESGGGLVQPGGSLRLSCAASGYIYSAYCMGWFRQAPGKGLVGVAAISIGGGSTYYADSVKGRFTISRDNSKNTLYLQMNSLRAEDTAVYYCAADVIPTETCLGGSWSGPFGYWGQGTLVTVSS

SEQ ID NO: 28 - protein, 128 aa, synthetic construct
  REGION 1..128: note = CTLA4 ISVD-29
QVQLVESGGGLVQPGGSLRLSCAASGYIYSAYCMGWFRQAPGKGLEGVAAISIGGGSTYYADSVKGRFTISRDNSKNTLYLQMNSLRAEDTAVYYCAADVIPTETCLGGSWSGPFGYWGQGTLVTVSS

SEQ ID NO: 29 - protein, 128 aa, synthetic construct
  REGION 1..128: note = CTLA4 ISVD-30
QVQLVESGGGLVQPGGSLRLSCAASGYIYSAYCMGWFRQAPGKEREGVAAISIGGGSTYYADSVKGRFTISRDNSKNTLYLQMNSLRAEDTAVYYCAADVIPTETCLGGSWSGPFGYWGQGTLVTVSS

SEQ ID NO: 30 - protein, 128 aa, synthetic construct
  REGION 1..128: note = CTLA4 ISVD-31
QVQLVESGGGLVQPGGSLRLSCAASGYIYSAYCMGWFRQAPGKEREGVAAIYIGGGSTYYADSVKGRFTISQDNSKNTLYLQMNSLRAEDTAVYYCAADVIPTETCLGGSWSGPFGYWGQGTLVTVSS

SEQ ID NO: 31 - protein, 128 aa, synthetic construct
  REGION 1..128: note = CTLA4 ISVD-32
QVQLVESGGGLVQPGGSLRLSCAASGYIYSAYCMGWFRQAPGKEREGVAAIYIGGGSTYYADSVKGRFTISRDNSKNTLYLQMNSLRAEDTAVYYCAADVIPTETCLGGSWSGPFGYWGQGTLVTVSS

SEQ ID NO: 32 - protein, 128 aa, synthetic construct
  REGION 1..128: note = CTLA4 ISVD-33
QVQLVESGGGLVQPGGSLRLSCAASGYIYSAYCMGWFRQAPGKGLVGVAAIYIGGGSTYYADSVKGRFTISRDNSKNTLYLQMNSLRAEDTAVYYCAADVIPTETCLGGSWSGPFGYWGQGTLVTVSS

SEQ ID NO: 33 - skipped

SEQ ID NO: 34 - protein, 15 aa, synthetic construct
  REGION 1..15: note = long linker
GGGGSGGGGSGGGGS

SEQ ID NO: 35 - protein, 232 aa, synthetic construct
  REGION 1..232: note = IgGl-Fc region with hinge
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 330 aa, synthetic construct [sequence omitted]

SEQ ID NO: 37 - protein, 124 aa, synthetic construct
  REGION 1..124: note = control dAb
QVQLQESGGGSVQAGGSLRLSCEASGYTYCMGWFRQAPGKEREGVALAVTGISIPYYADSVKGRFTISQDNVKNTLYLQMNSLKPEDAAMYYCAASTIRYVCPGLNRGDQFKNWGQGTQVTVSS

SEQ ID NO: 38 - protein, 232 aa, synthetic construct
  REGION 1..232: note = IgGl-Fc region with hinge (C-S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 227 aa, synthetic construct
  REGION 1..227: note = human IgG1-Fc region without hinge
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 493 aa, synthetic construct
  REGION 1..493: note = aPDL1.9-aCTLA4.34-Fc
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSGAPQVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 505 aa, synthetic construct
  REGION 1..505: note = aPDL1.9-L-aCTLA4.34-Fc
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSGGGGSGGGGSGGGGSQVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 493 aa, synthetic construct
  REGION 1..493: note = aCTLA4.34-aPDL1.9-Fc
QVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSGAPQVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 505 aa, synthetic construct
  REGION 1..505: note = aCTLA4.34-L-aPDL1.9-FC
QVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSGGGGSGGGGSGGGGSQVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 487 aa, synthetic construct
  REGION 1..487: note = aPDL1.9-dAb-Fc
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSGAPQVQLQESGGGSVQAGGSLRLSCEASGYTYCMGWFRQAPGKEREGVALAVTGISIPYYADSVKGRFTISQDNVKNTLYLQMNSLKPEDAAMYYCAASTIRYVCPGLNRGDQFKNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 487 aa, synthetic construct
  REGION 1..487: note = dAb-aCTLA4.34-Fc
QVQLQESGGGSVQAGGSLRLSCEASGYTYCMGWFRQAPGKEREGVALAVTGISIPYYADSVKGRFTISQDNVKNTLYLQMNSLKPEDAAMYYCAASTIRYVCPGLNRGDQFKNWGQGTQVTVSSGAPQVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 491 aa, synthetic construct
  REGION 1..491: note = aPDL1.6-aCTLA4.34-Fc
QVQLQESGGGSVQAGGSLRLSCAASGNIIRVRCMAWFRQAPGKERERGPNILTTTISTYLADSVKGRFTISQNNAKSTVYLQMNSLKPEDTAMYYCAADSFGYPTCPGPASSGAFQYWGQGTQVTVSSGAPQVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 487 aa, synthetic construct
  REGION 1..487: note = aPDL1.6-dAb-Fc
QVQLQESGGGSVQAGGSLRLSCAASGNIIRVRCMAWFRQAPGKERERGPNILTTTISTYLADSVKGRFTISQNNAKSTVYLQMNSLKPEDTAMYYCAADSFGYPTCPGPASSGAFQYWGQGTQVTVSSGAPQVQLQESGGGSVQAGGSLRLSCEASGYTYCMGWFRQAPGKEREGVALAVTGISIPYYADSVKGRFTISQDNVKNTLYLQMNSLKPEDAAMYYCAASTIRYVCPGLNRGDQFKNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 491 aa, synthetic construct
  REGION 1..491: note = aPDL1.m3-aCTLA4.34-Fc
QVQLQESGGGLVQPGGSLRLSCAASGKMSSRRCMAWFRQAPGKERERVADVNPNSGGSIYNQRFKGRFTISQNNAKSTVYLQMNSLKPEDTAMYYCAADSFEDPTCTLVTSSGAFQYWGQGTQVTVSSGAPQVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 487 aa, synthetic construct
  REGION 1..487: note = aPDL1.m3-dAb-Fc
QVQLQESGGGLVQPGGSLRLSCAASGKMSSRRCMAWFRQAPGKERERVADVNPNSGGSIYNQRFKGRFTISQNNAKSTVYLQMNSLKPEDTAMYYCAADSFEDPTCTLVTSSGAFQYWGQGTQVTVSSGAPQVQLQESGGGSVQAGGSLRLSCEASGYTYCMGWFRQAPGKEREGVALAVTGISIPYYADSVKGRFTISQDNVKNTLYLQMNSLKPEDAAMYYCAASTIRYVCPGLNRGDQFKNWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 491 aa, synthetic construct
  REGION 1..491: note = aPDL1.9-aCTLA4.13-Fc
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSGAPQVQLQESGGGSVQAGGSLRLSCAASGYIYSAYCMGWFRQAPGKERVGVAAISIGGGSTYYADSVKGRFTISQDNAKNTLYLQMNSLKPEDTAMYYCAADVIPTETCLGGSWSGPFGYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 487 aa, synthetic construct
  REGION 1..487: note = dAb-aCTLA4.13-Fc
QVQLQESGGGSVQAGGSLRLSCEASGYTYCMGWFRQAPGKEREGVALAVTGISIPYYADSVKGRFTISQDNVKNTLYLQMNSLKPEDAAMYYCAASTIRYVCPGLNRGDQFKNWGQGTQVTVSSGAPQVQLQESGGGSVQAGGSLRLSCAASGYIYSAYCMGWFRQAPGKERVGVAAISIGGGSTYYADSVKGRFTISQDNAKNTLYLQMNSLKPEDTAMYYCAADVIPTETCLGGSWSGPFGYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 357 aa, synthetic construct
  REGION 1..357: note = CTLA4-muFc
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQGSMDPKSSDKTHTCPPCPAPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 53 - protein, 452 aa, synthetic construct
  REGION 1..452: note = PD-L1-muFc
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREENLFNVTSTLRINTTTNEIFYCTFRRLDPEENHTAELVIPELPLAHPPNERTGSMDPKSSDKTHTCPPCPAPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 54 - protein, 448 aa, synthetic construct
  REGION 1..448: note = PD-L1-huFc
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREENLFNVTSTLRINTTTNEIFYCTFRRLDPEENHTAELVIPELPLAHPPNERTDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 442 aa, synthetic construct
  REGION 1..442: note = CD80-muFc
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNIEGRMDPKSSDKTHTCPPCPAPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 56 - protein, 357 aa, synthetic construct
  REGION 1..357: note = CTLA4-huFc
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 449 aa, synthetic construct
  REGION 1..449: note = CD86-muFc
YFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPGSMDPKSSDKTHTCPPCPAPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 58 - protein, 381 aa, synthetic construct
  REGION 1..381: note = PD1-muFc
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQIEGRMDPKSSDKTHTCPPCPAPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 59 - protein, 448 aa, synthetic construct
  REGION 1..448: note = Ipilimumab HC
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 215 aa, synthetic construct
  REGION 1..215: note = Ipilimumab LC
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 451 aa, synthetic construct
  REGION 1..451: note = Durvalumab HC
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 215 aa, synthetic construct
  REGION 1..215: note = Durvalumab LC
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 220 aa, synthetic construct
  REGION 1..220: note = human PD-L1
TVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREENLFNVTSTLRINTTTNEIFYCTFRRLDPEENHTAELVIPELPLAHPPNERT

SEQ ID NO: 64 - protein, 127 aa, synthetic construct
  REGION 1..127: note = N-terminal IgV domain of human PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRIT

SEQ ID NO: 65 - protein, 214 aa, synthetic construct
  REGION 1..214: note = CLCL1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, synthetic construct
  REGION 1..214: note = CLCL2
DIQMTQSPSSLSASVGDRVTITCRASQDVNIAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 214 aa, synthetic construct
  REGION 1..214: note = CLCL3
DIQMTQSPSSLSASVGDRVTITCRASQDVNTAVAWYQQKPGKAPKLLIYSASFLYSGVPSRFSGSRSGTDFTLTISSLQPEDFATYYCQQHYTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 214 aa, synthetic construct
  REGION 1..214: note = CLCL4
DIQMTQSPSSLSASVGDRVTITCRASQDVNIAVAWYQQKPGKAPKLLIYSASFLYSGVPSRFSGSRSGTDFTLTISSLQPEDFATYYCQQHYTYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 214 aa, synthetic construct
  REGION 1..214: note = CLCL5
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 214 aa, synthetic construct
  REGION 1..214: note = CLCL6
DIQMTQSPSSLSASVGDRVTITCKASQDVSIGVAWYQQKPGKAPKLLIYSASYRYTGVPSRFSGSGSGTDFTLTISSLQPEDFATYYCQQYYITPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 642 bp, synthetic construct
  misc_feature 1..642: note = CLCL1 gene
  source 1..642: mol_type = other DNA
gacatccagatgacccagagccccagcagcctgagcgccagcgtgggcgaccgcgtgaccatcacctgccgcgccagccaggacgtgaacactgccgttgcatggtaccagcagaagcccggcaaggcccccaagctgctgatctacagcgccagcttcctgtacagcggcgtgcccagccgcttcagcggcagccgcagcggcaccgacttcaccctgaccatcagcagcctgcagcccgaggacttcgccacctactactgccagcagcactatactactcctccaacattcggccagggcaccaaggtggagatcaagcgcaccgtggccgcccccagcgtgttcatcttcccccccagcgacgagcagctcaagagcggcaccgccagcgtggtgtgcctgctgaacaacttctacccccgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccgcggcgagtgc

SEQ ID NO: 72 - DNA, 642 bp, synthetic construct
  misc_feature 1..642: note = CLCL2 gene
  source 1..642: mol_type = other DNA
gacatccagatgacccagagccccagcagcctgagcgccagcgtgggcgaccgcgtgaccatcacctgccgcgccagccaggacgtgaacattgccgttgcatggtaccagcagaagcccggcaaggcccccaagctgctgatctacagcgccagcttcctgtacagcggcgtgcccagccgcttcagcggcagccgcagcggcaccgacttcaccctgaccatcagcagcctgcagcccgaggacttcgccacctactactgccagcagcactatactactcctccaacattcggccagggcaccaaggtggagatcaagcgcaccgtggccgcccccagcgtgttcatcttcccccccagcgacgagcagctcaagagcggcaccgccagcgtggtgtgcctgctgaacaacttctacccccgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccgcggcgagtgc

SEQ ID NO: 73 - DNA, 642 bp, synthetic construct
  misc_feature 1..642: note = CLCL3 gene
  source 1..642: mol_type = other DNA
gacatccagatgacccagagccccagcagcctgagcgccagcgtgggcgaccgcgtgaccatcacctgccgcgccagccaggacgtgaacactgccgttgcatggtaccagcagaagcccggcaaggcccccaagctgctgatctacagcgccagcttcctgtacagcggcgtgcccagccgcttcagcggcagccgcagcggcaccgacttcaccctgaccatcagcagcctgcagcccgaggacttcgccacctactactgccagcagcactatacttatcctccaacattcggccagggcaccaaggtggagatcaagcgcaccgtggccgcccccagcgtgttcatcttcccccccagcgacgagcagctcaagagcggcaccgccagcgtggtgtgcctgctgaacaacttctacccccgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccgcggcgagtgc

SEQ ID NO: 74 - DNA, 642 bp, synthetic construct
  misc_feature 1..642: note = CLCL4 gene
  source 1..642: mol_type = other DNA
gacatccagatgacccagagccccagcagcctgagcgccagcgtgggcgaccgcgtgaccatcacctgccgcgccagccaggacgtgaacattgccgttgcatggtaccagcagaagcccggcaaggcccccaagctgctgatctacagcgccagcttcctgtacagcggcgtgcccagccgcttcagcggcagccgcagcggcaccgacttcaccctgaccatcagcagcctgcagcccgaggacttcgccacctactactgccagcagcactatacttatcctccaacattcggccagggcaccaaggtggagatcaagcgcaccgtggccgcccccagcgtgttcatcttcccccccagcgacgagcagctcaagagcggcaccgccagcgtggtgtgcctgctgaacaacttctacccccgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccgcggcgagtgc

SEQ ID NO: 75 - DNA, 642 bp, synthetic construct
  misc_feature 1..642: note = CLCL5 gene
  source 1..642: mol_type = other DNA
gacatccagatgacccagagccccagcagcctgagcgccagcgtgggcgaccgcgtgaccatcacctgcaaggccagccaggacgtgagcatcggcgtggcctggtaccagcagaagcccggcaaggcccccaagctgctgatctacagcgccagctaccgctacaccggcgtgcccagccgcttcagcggcagcggcagcggcaccgacttcaccctgaccatcagcagcctgcagcccgaggacttcgccacctactactgccagcagtactacatctacccctacaccttcggccagggcaccaaggtggagatcaagcgcaccgtggccgcccccagcgtgttcatcttcccccccagcgacgagcagctcaagagcggcaccgccagcgtggtgtgcctgctgaacaacttctacccccgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccgcggcgagtgc

SEQ ID NO: 76 - DNA, 642 bp, synthetic construct
  misc_feature 1..642: note = CLCL6 gene
  source 1..642: mol_type = other DNA
gacatccagatgacccagagccccagcagcctgagcgccagcgtgggcgaccgcgtgaccatcacctgcaaggccagccaggacgtgagcatcggcgtggcctggtaccagcagaagcccggcaaggcccccaagctgctgatctacagcgccagctaccgctacaccggcgtgcccagccgcttcagcggcagcggcagcggcaccgacttcaccctgaccatcagcagcctgcagcccgaggacttcgccacctactactgccagcagtactacatcaccccctacaccttcggccagggcaccaaggtggagatcaagcgcaccgtggccgcccccagcgtgttcatcttcccccccagcgacgagcagctcaagagcggcaccgccagcgtggtgtgcctgctgaacaacttctacccccgcgaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccgcggcgagtgc

SEQ ID NO: 77 - protein, 630 aa, synthetic construct
  REGION 1..630: note = Her2 m1
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGACTLVCPLANQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 78 - protein, 630 aa, synthetic construct
  REGION 1..630: note = Her2 m2
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPAADQCVACAHYKDPAFCVARCPSGVKPDLSYMPIWKFPDEEGACQPCPINCTHSCVDLDDKGCPAEQRASPLT

SEQ ID NO: 79 - protein, 630 aa, synthetic construct
  REGION 1..630: note = Her2 m3
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDAAFCVARCPSGVKPDLSYMPIWKFPDEEGACQPCPINCTHSCVDLDDKGCPAEQRASPLT

SEQ ID NO: 80 - DNA, 1350 bp, synthetic construct
  misc_feature 1..1350: note = Tmab Heavy chain
  source 1..1350: mol_type = other DNA
gaggtgcagctcgtggagagcggcggcggcctggtgcagcccggcggcagcctgcgcctgagctgcgccgccagcggcttcaacatcaaggacacctacatccactgggtgcgccaggcccccggcaagggcctggagtgggtggcccgcatctaccccaccaacggctacacccgctacgccgacagcgtgaagggccgcttcaccatcagcgccgacaccagcaagaacaccgcctacctgcagatgaacagcctgcgcgccgaggacaccgccgtgtactactgcagccgctggggcggcgacggcttctacgccatggactactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttctcgtgcagcgtgatgcacgaggccctgcacaaccactacacccagaagagcctgagcctgagccccggcaag

SEQ ID NO: 81 - DNA, 1347 bp, synthetic construct
  misc_feature 1..1347: note = Pmab Heavy chain
  source 1..1347: mol_type = other DNA
gaggtgcagctcgtggagagcggcggcggcctggtgcagcccggcggcagcctgcgcctgagctgcgccgccagcggcttcaccttcaccgactacaccatggactgggtgcgccaggcccccggcaagggcctggagtgggtggccgacgtgaaccccaacagcggcggcagcatctacaaccagcgcttcaagggccgcttcaccctgagcgtggaccgcagcaagaacaccctgtacctgcagatgaacagcctgcgcgccgaggacaccgccgtgtactactgcgcccgcaacctgggccccagcttctacttcgactactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttctcgtgcagcgtgatgcacgaggccctgcacaaccactacacccagaagagcctgagcctgagccccggcaag

SEQ ID NO: 82 - protein, 630 aa, synthetic construct
  REGION 1..630: note = HER2 ECD
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 83 - protein, 450 aa, synthetic construct
  REGION 1..450: note = Tmab HC-hole
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 449 aa, synthetic construct
  REGION 1..449: note = Pmab HC-hole
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 85 - protein, 450 aa, synthetic construct
  REGION 1..450: note = Tmab HC-mix1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 449 aa, synthetic construct
  REGION 1..449: note = Pmab-CLC1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLKSDGSFFLYSDLTVDKSRWQQGNVFSCSVMHEALHNHYTQKSLSLSPGK

SEQ ID NO: 87 - protein, 120 aa, synthetic construct [sequence omitted]

SEQ ID NO: 88 - protein, 119 aa, synthetic construct [sequence omitted]

SEQ ID NO: 89 - protein, 227 aa, synthetic construct
  REGION 1..227: note = Fc1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 227 aa, synthetic construct
  REGION 1..227: note = Fc2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 107 aa, synthetic construct [sequence omitted]

SEQ ID NO: 92 - protein, 107 aa, synthetic construct
  REGION 1..107: note = CLC2 VL
DIQMTQSPSSLSASVGDRVTITCRASQDVNIAVAWYQQKPGKAPKLLIYSASFLYSGVPSRFSGSRSGTDFTLTISSLQPEDFATYYCQQHYTTPPTFGQGTKVEIK

SEQ ID NO: 93 - protein, 107 aa, synthetic construct
  REGION 1..107: note = CLC3 VL
DIQMTQSPSSLSASVGDRVTITCRASQDVNTAVAWYQQKPGKAPKLLIYSASFLYSGVPSRFSGSRSGTDFTLTISSLQPEDFATYYCQQHYTYPPTFGQGTKVEIK

SEQ ID NO: 94 - protein, 107 aa, synthetic construct
  REGION 1..107: note = CLC4 VL
DIQMTQSPSSLSASVGDRVTITCRASQDVNIAVAWYQQKPGKAPKLLIYSASFLYSGVPSRFSGSRSGTDFTLTISSLQPEDFATYYCQQHYTYPPTFGQGTKVEIK

SEQ ID NO: 95 - protein, 107 aa, synthetic construct [sequence omitted]

SEQ ID NO: 96 - protein, 107 aa, synthetic construct
  REGION 1..107: note = CLC6 VL
DIQMTQSPSSLSASVGDRVTITCKASQDVSIGVAWYQQKPGKAPKLLIYSASYRYTGVPSRFSGSGSGTDFTLTISSLQPEDFATYYCQQYYITPYTFGQGTKVEIK

SEQ ID NO: 97 - protein, 449 aa, synthetic construct
  REGION 1..449: note = heavy chain 1-1
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 98 - protein, 450 aa, synthetic construct
  REGION 1..450: note = heavy chain 2-1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 99 - protein, 449 aa, synthetic construct
  REGION 1..449: note = heavy chain 1-2
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 100 - protein, 450 aa, synthetic construct
  REGION 1..450: note = heavy chain 2-2
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 101 - protein, 227 aa, synthetic construct
  REGION 1..227: note = Fc 1-1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 102 - protein, 227 aa, synthetic construct
  REGION 1..227: note = Fc 2-1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 103 - protein, 227 aa, synthetic construct
  REGION 1..227: note = Fc 1-2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 104 - protein, 227 aa, synthetic construct; identical to SEQ ID NO: 90
  REGION 1..227: note = Fc 2-2